(19) United States
(12) Patent Application Publication
(10) Pub. No.: US 2010/0216751 A1
(43) Pub. Date: Aug. 26, 2010

(54) Liquid Bevirimat Dosage Forms for Oral Administration

(75) Inventors: Jules S. Jacob, Yardley, PA (US); John Richards, Acton, MA (US); John G. Augustine, Lynn, MA (US); Jaqueline S. Milea, Boston, MA (US)

Correspondence Address: Myriad PHARMACEUTICALS, Inc.;c/o CPA Global, P.O. Box 52050, Minneapolis, MN, 55402, US

(73) Assignee: Myriad Pharmaceuticals, Inc., Salt Lake City, UT (US)

(21) Appl. No.: 12/731,786

(22) Filed: Mar. 25, 2010

Related U.S. Application Data
(63) Continuation of application No. PCT/US2008/011094, filed on Sep. 25, 2008.
(60) Provisional application No. 60/975,027, filed on Sep. 25, 2007.

(51) Int. Cl. A61K 31/56

(52) U.S. Cl. 514/169

(57) ABSTRACT

The present application discloses and claims liquid pharmaceutical compositions comprising bevirimat dimeglumine as a drug substance, methods of treatment comprising administering such compositions to a subject in need thereof, and the use of such compositions in the manufacture of medicaments.

### CROSS-REFERENCE TO RELATED APPLICATIONS

This application is a continuation of International Application No. PCT/US2008/011094, filed Sep. 25, 2008, and published as WO 2009/042166, which claims the benefit of U.S. Provisional Application Ser. No. 60/975,027, filed Sep. 25, 2007; both of which are incorporated by reference herein in their entirety.

### BACKGROUND OF THE INVENTION

1. Field of the Invention

The present invention relates to liquid oral pharmaceutical compositions comprising a bevirimat drug substance. Specifically, the present invention relates to liquid oral pharmaceutical compositions comprising the compound bevirimat dimeglumine as a drug substance, methods of treatment comprising administering such compositions to a subject in need thereof, and the use of such compositions in the manufacture of medicaments.

2. Background Art

U.S. Pat. No. 5,679,828 mentions betulinic acid and dihydrobetulinic acid derivatives, including 3-O-(3′,3′-dimethylsuccinyl)betulinic acid (also known as (3β)-3-(carboxy-3-methyl-1-oxobutoxy)-lup-20(29)-en-28-oic acid, DSB, and bevirimat) (structure shown below), as potent anti-HIV agents.

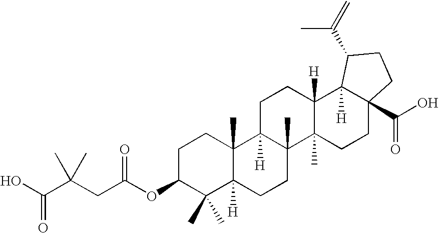

U.S. Pat. No. 6,642,217 mentions the use of betulin and analogs thereof for treating fungal and yeast infections.

U.S. Patent Application Publication No. US 2005/0239748 mentions N-methylglucamine, potassium, and sodium pharmaceutical salts of 3-O-(3′,3′-dimethylsuccinyl)betulinic acid that are useful in the treatment of HIV infection and related diseases. The di-N-methylglucamine salt of 3-O-(3′,3′-dimethylsuccinyl)betulinic acid is also referred to as “bevirimat dimeglumine” or “bevirimat-2NMG” and has the following structure:

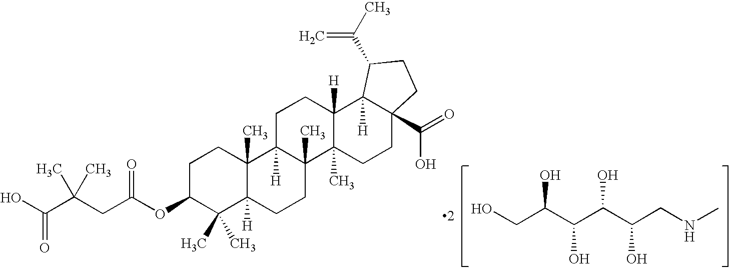

U.S. Patent Application Publication No. US 2006/0252704 A1 (U.S. patent application Ser. No. 11/401,960) mentions several solid state forms of bevirimat dimeglumine including Form I, Form II and amorphous.

Bevirimat and its salts prevent viral replication by specifically and potently inhibiting cleavage of the capsid protein (“CA”) from the SP1 spacer protein (“SP1”). By specifically inhibiting cleavage of the CA-SP1 protein, the capsid is prevented from condensing into its mature, conical shape that is considered essential for HIV-1 virions to be infectious. Bevirimat is the first compound to target this particular step in the viral life cycle.

After administration, unformulated bevirimat, or a salt thereof, is not well absorbed from the gastrointestinal tract into the blood. Accordingly, a need exists for suitable bevirimat dosage forms. Inferior properties of unformulated bevirimat and its salts and other known compositions comprising bevirimat or a salt thereof include, but are not limited to, one or more of the following:

(1) inadequate bioavailability;
(2) poor solubility of the pharmaceutical composition in gastric fluid;
(3) insufficient dispersion of bevirimat in gastric fluid;
(4) suboptimal long term safety profile for oral dosage forms;
(5) suboptimal long term chemical stability of the final oral dosage form;
(6) suboptimal long term physical stability of the final oral dosage form;
(7) tendency for conversion to metastable forms, including colloidal gels, of bevirimat;
(8) lengthy dissolution times for oral dosage forms; and
(9) precipitation in gastric or intestinal fluids.

Some challenges with formulating bevirimat involve its unique physico-chemical properties. Bevirimat is a high molecular weight di-acid with low solubility in aqueous media, a high ClogP, and exhibits a high fraction of protein binding (99+%) to human serum albumin.

Many formulations containing a bevirimat drug substance form colloidal gels that are unsuitable for oral administration. Other bevirimat formulations begin to precipitate, gel or crystallize in solution or upon introduction to either simulated gastric fluid (“SGF”) or simulated intestinal fluid (“SIF”). One challenge associated with the formulation of bevirimat is to develop a formulation that dissolves into a free-flowing solution and disperses evenly in test solutions, including SGF and SIF.

Another challenge associated with bevirimat's unique physico-chemical properties is that bevirimat, including its salts, precipitates and gels from many formulations when introduced to aqueous media or SGF and SIF.

Another challenge with formulating bevirimat and its salts involves the provision of a formulation suitable for long term dosing. Liquid formulations of bevirimat dimeglumine have been investigated in human clinical trials as a 10% (w/v) hydroxypropyl-β-cyclodextrin (“HPBCD”) orally administered solution. While the tested HPBCD formulation delivered acceptable blood plasma levels of bevirimat in multiple Phase I and Phase IIa trials, the dosing requirements of those trials were relatively short compared to the extended dosing required by HIV/AIDS patients. Many regulatory agencies, including the U.S. Food & Drug Administration, generally do not approve formulations comprising HPBCD for indications that require dosing for longer than one month. Though a 200 mg HPBCD formulation achieved a mean trough concentration 33.8 μg/mL of bevirimat in human clinical trials, a 400 mg bevirimat formulation free of HPBCD achieved a mean trough concentration of only 19.9 mg/mL.

Another challenge with formulating bevirimat and its salts involves identifying a dose that provides a plasma concentration suitable to achieve a 90% inhibition of viral replication rates. Doses for the Phase I and Phase IIa clinical studies of bevirimat, selected based on pre-clinical studies in animals, ranged from 25 mg-200 mg of bevirimat. Subsequent modeling of the human dose-response relationship for bevirimat suggested that the EC₉₀ for bevirimat is about 350 to about 400 mg of bevirimat.

Another challenge with formulating bevirimat salts is that the administration of bevirimat salts effects the precipitation of solids including bevirimat into the gastric fluid. The addition of salts to gastric fluid initiates a competition between different solutes, e.g. the free acid of the active pharmaceutical ingredient (“API”) and counterions, for solvent molecules. This competition is influenced by the size, charge and valencies of each solute. As triterpene derivatives have very high size: charge ratios relative to the counter ions, the administration of pharmaceutical compositions comprising triterpene salts often leads to the precipitation of the dissociated API from gastric fluid, also referred to as “salting-out.”

The physical and chemical properties of bevirimat and its salts have presented numerous challenges to the development of a bevirimat formulation suitable for long term dosing. Bevirimat is poorly soluble in 100% water, 100% saline, 100% dextrose, 100% PEG 400, and 25% Poloxamer 188. Bevirimat dimeglumine has a limited solubility in 100% water, 100% saline, 100% dextrose, 100% PEG 400, and 25% Poloxamer 188.

Another challenge with formulating bevirimat and its salts involves the need for a formulation that exhibits a long term stability profile, for example a formulation that is stable over 2 years under normal storage conditions, or a formulation that is stable for 6 months at 40° C. at 75% relative humidity (RH).

Due in part to bevirimat's and its salts' low water solubility, bevirimat, when formulated by traditional techniques, exhibits an erratic dissolution profile in gastrointestinal fluids, which consequently results in variable pharmacokinetics and oral bioavailability. In addition, bevirimat compositions with water are not stable during storage.

A need continues to exist for liquid dosage forms containing bevirimat or a salt thereof for oral administration, wherein the dosage forms remain both chemically and physically stable during storage.

### BRIEF SUMMARY OF THE INVENTION

It has been discovered that pharmaceutical compositions comprising a bevirimat drug substance, and especially bevirimat-2NMG, and certain pharmaceutically acceptable carrier materials can deliver a therapeutically effective amount of bevirimat to the patient. These combinations of the bevirimat drug substance and carrier materials have been found to possess improved bioavailability, chemical stability, physical stability, dispersion into SGF and SIF, safety, as well as other improved pharmacokinetic, chemical or physical properties. The present invention provides these pharmaceutical compositions, unit dosage forms based thereon, and methods for the preparation and use of both.

In some embodiments, the present invention provides an orally deliverable liquid pharmaceutical composition comprising (a) a bevirimat drug substance, and (b) a solvent liquid comprising (i) at least one pharmaceutically acceptable solvent, and (ii) a polymer, wherein the polymer is present in an amount sufficient to retard gelling and precipitation of the bevirimat in simulated gastric fluid over 48 hours at 37° C.

In one aspect, the present invention provides an orally deliverable liquid pharmaceutical composition, comprising a bevirimat drug substance, preferably bevirimat-2NMG, propylene glycol, ethanol, and glycerin. The composition of the present invention can optionally contain one or more flavoring agents, and optionally a sweetener. Preferably, the weight/volume percent ratio of ethanol:propylene glycol:glycerin is about 1:from about 2 to about 4:about 1. Preferably, the composition comprises from about 26 to about 38 w/v-% of propylene glycol, from about 10 to about 15 w/v-% of ethanol, and from about 10 to about 17 w/v-% of glycerin. More preferably, the composition comprises from about 27 to about 37 w/v-% of propylene glycol, from about 11 to about 14 w/v-% of ethanol, and from about 11 to about 16 w/v-% of glycerin. Advantageously, the composition comprises from about 32.6 to about 36 w/v-% of propylene glycol, from about 12.4 to about 13.6 w/v-% of ethanol, and from about 13.2 to about 14.6 w/v-% of glycerin. Preferably, the orally deliverable liquid pharmaceutical composition comprises bevirimat-2NMG as the bevirimat drug substance. Preferably, the orally deliverable liquid pharmaceutical composition comprises from about 0.01 to about 0.42 w/v-% of bevirimat-2NMG, more preferably from about 0.08 to about 0.35 w/v-% of bevirimat-2NMG, and advantageously from about 0.18 to about 0.25 w/v-% of bevirimat-2NMG.

Incorporating buffer (preferably phosphate buffer, and more preferably sodium phosphate buffer) into the orally deliverable liquid pharmaceutical composition of the present invention improves the physical stability of the composition. Accordingly, one aspect of the present invention provides an orally deliverable liquid pharmaceutical composition, comprising a bevirimat drug substance, preferably bevirimat-2NMG, propylene glycol, ethanol, glycerin, and buffer providing a pH of from about 7.0 to about 9.0, and optionally one or more flavoring agents. The buffer is present in an amount effective to maintain a pH of from about 7.0 to about 9.0, the final molarity of the buffer being from about 0.01M to about 0.4M, preferably from about 0.05M to about 0.3M, and more preferably from about 0.1 to about 0.2M. Suitable buffers are any buffers that do not react with the bevirimat drug substance, and preferably phosphate buffers, especially sodium phosphate buffer. Preferably, the composition comprises 0.2 M phosphate buffer, and more preferably the composition comprises 0.2 M sodium phosphate buffer, pH 7.9. Preferably, the composition comprises from 20 to about 31 w/v-% of 0.2 M phosphate buffer, pH 7.9; more preferably from about 22 to about 30 w/v-% of 0.2 M phosphate buffer, pH 7.9, and advantageously from about 26.2 to about 29 w/v-% of phosphate buffer, pH 7.9.

The one or more flavoring agents are present in the orally deliverable liquid pharmaceutical compositions in an amount effective to provide a desirable taste characteristic.

Further, it has been found that some flavoring agents can contribute to the chemical instability of the orally deliverable liquid pharmaceutical compositions of the present invention, whereas some flavoring agents do not negatively affect the chemical stability. Thus, suitable flavoring agents include those that do not negatively affect the chemical stability of the pharmaceutical composition of the present invention. In this aspect, the present invention provides an orally deliverable liquid pharmaceutical composition, comprising a bevirimat drug substance, preferably bevirimat-2NMG, propylene glycol, ethanol, glycerin, buffer providing a pH of from about 7.0 to about 9.0 (preferably phosphate buffer), and one or more flavoring agents selected from the group consisting of mint, menthol, peach, Dulce de Leche, coffee, orange, orange/vanilla, strawberry, peach/mango, punch, chocolate, mixed perry, mocha, strawberry/banana, and banana. Preferably, the one or more flavoring agents comprise mint, menthol, or combinations thereof. Useful compositions of the present invention include those, wherein the one or more flavoring agents are present in a total amount of from about 0.1 to about 1.5 w/v-%, preferably from about 0.2 to about 1.3 w/v-%, and more preferably from about 0.25 to about 1.2 w/v-%. Preferably, the orally deliverable liquid pharmaceutical composition comprises from about 0.4 to about 0.7 w/v-% menthol or from about 0.1 to about 0.8 w/v-% of mint; more preferably from about 0.45 to about 0.65 w/v-% of menthol or from about 0.2 to about 0.6 w/v-% of mint; and advantageously from about 0.53 to about 0.60 w/v-% of menthol or from about 0.25 to about 0.50 w/v-% of mint.

Further, the present invention provides an orally deliverable liquid pharmaceutical composition, comprising from about 0.01 to about 0.42 w/v-% of bevirimat dimeglumine; from about 10 to about 15 w/v-% of ethanol; from about 26 to about 38 w/v-% of propylene glycol; from about 10 to about 17 w/v-% of glycerin; from about 20 to about 31 w/v-% of 0.2 M phosphate buffer (especially 0.2 M sodium phosphate buffer), pH 7.9; from about 4 to about 7 w/v-% of xylitol; from about 0.4 to about 0.7 w/v-% of menthol or from about 0.1 to about 0.8 w/v-% of mint; and sterilized water q.s. to from about 30 to about 54 w/v-%.

The present invention also provides an orally deliverable liquid pharmaceutical composition, comprising from about 0.08 to about 0.35 w/v-% of bevirimat dimeglumine; from about 11 to about 14 w/v-% of ethanol; from about 27 to about 37 w/v-% of propylene glycol; from about 11 to about 16 w/v-% of glycerin; from about 22 to about 30 w/v-% of 0.2 M phosphate buffer (especially 0.2 M sodium phosphate buffer), pH 7.9; from about 4.5 to about 6.5 w/v-% of xylitol; from about 0.45 to about 0.65 w/v-% of menthol or from about 0.2 to about 0.6 w/v-% of mint; and sterilized water q.s. to from about 32 to about 48 w/v-%.

Furthermore, the present invention provides an orally deliverable liquid pharmaceutical composition, comprising from about 0.18 to about 0.25 w/v-% of bevirimat dimeglumine; from about 12.4 to about 13.6 w/v-% of ethanol; from about 32.6 to about 36.0 w/v-% of propylene glycol; from about 13.2 to about 14.6 w/v-% of glycerin; from about 26.2 to about 29.0 w/v-% of 0.2 M phosphate buffer (especially 0.2 M sodium phosphate buffer), pH 7.9; from about 5.25 to about 6.0 w/v-% of xylitol; from about 0.53 to about 0.60 w/v-% of menthol or from about 0.25 to about 0.50 w/v-% of mint; and sterilized water q.s. to about 33.0-40.0 w/v-%.

The present invention relates to a method of treating a medical condition or disorder in a subject where treatment with a HIV-1 maturation inhibitor is indicated, comprising orally administering to the subject a composition of the present invention.

The present invention is also directed to to the use of a bevirimat drug substance, preferably bevirimat-2NMG (or bevirimat dimeglumine), in the manufacture of an orally deliverable liquid pharmaceutical composition for treating HIV infection, and specifically HIV-1 infection, and related diseases.

Additional embodiments and advantages of the invention will be set forth in part of the description that follows, and in part, will be obvious from the description, or may be learned by practice of the invention. The embodiments and advantages of the present invention will be realized and attained by means of elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS/FIGURES

FIG. 1 is a graph depicting dose-normalized AUC of bevirimat liquid oral formulations 1-15, described in Table 5 of Example 19, in Cynomolgus monkey model.

FIG. 2 is a graph depicting the pharmacokinetics of liquid bevirimat formulations in Cynomolgous monkeys, i.e., the plasma concentrations of bevirimat free di-acid versus time following administration.

FIG. 3 is a graph depicting the average plasma concentration of bevirimat versus time following oral administration to male Cynomolgus monkeys described in Example 21.

FIG. 4 is a graph depicting the average plasma concentration of bevirimat versus time following oral administration to male Cynomolgus monkeys described in Example 22.

FIG. 5 is a graph depicting the average plasma concentration of bevirimat versus time following oral administration to male Cynomolgus monkeys in Example 23.

FIG. 6 is a graph depicting the average plasma concentration of bevirimat versus time following oral administration to male Cynomolgus monkeys described in Example 24.

FIG. 7 is a graph depicting the average plasma concentration of bevirimat versus time following oral administration to male Cynomolgus monkeys described in Example 25.

FIG. 8 is a graph depicting the average plasma concentration of bevirimat versus time following oral administration to male Cynomolgus monkeys described in Example 27.

FIG. 9 is a graph depicting the average plasma concentration of bevirimat versus time following oral administration to male Cynomolgus monkeys described in Example 28.

FIG. 10 is a graph depicting the average plasma concentration of bevirimat versus time following oral administration to male Cynomolgus monkeys described in Example 29.

FIG. 11 is a graph depicting the average plasma concentration of bevirimat versus time following oral administration to male Cynomolgus monkeys described in Example 30.

FIG. 12 is a graph depicting the average plasma concentration of bevirimat versus time following oral administration to male Cynomolgus monkeys described in Example 31.

FIG. 13 is a graph depicting the average plasma concentration of bevirimat versus time following oral administration to male Cynomolgus monkeys described in Example 32.

FIG. 14 is a graph depicting the average plasma concentration of bevirimat versus time following oral administration to male Cynomolgus monkeys described in Example 33.

FIG. 15 is a graph depicting the average plasma concentration of bevirimat versus time following oral administration to human volunteers, 0-336 hours, described in Example 34.

FIG. 16 is a graph depicting the average plasma concentration of bevirimat versus time following oral administration to human volunteers, 0-24 hours, described in Example 34.

FIG. 17 is a graph depicting the average plasma concentration of bevirimat versus time following oral administration to male Cynomolgus monkeys described in Example 35.

FIG. 18 is a graph depicting the average plasma concentration of bevirimat versus time following oral administration to male Cynomolgus monkeys described in Example 36.

FIG. 19 is a graph depicting the average plasma concentration of bevirimat versus time following oral administration to male Cynomolgus monkeys described in Example 37.

FIG. 20 is a graph depicting the average plasma concentration of bevirimat versus time following oral administration to male Cynomolgus monkeys described in Example 38.

### DETAILED DESCRIPTION OF THE INVENTION

It has been discovered that orally deliverable liquid pharmaceutical compositions comprising bevirimat drug substance as the active ingredient in a daily dosage amount of about 100 mg to about 1000 mg and a pharmaceutically acceptable carrier material exhibit superior properties, including activity, potency, safety and therapeutic effectiveness within this dosage range. These compositions deliver bevirimat to a patient at therapeutically effective concentrations sufficient to inhibit replication of HIV-1 while maintaining an acceptable safety profile. In one embodiment, the composition of the present invention is administered only once per day. In one embodiment, the composition of the present invention is administered twice per day. Undesirable side effects such as, but not limited to, gastrointestinal irritation, unpleasant taste, and carcinogenicity are avoided or minimized with the pharmaceutical compositions of the present invention.

Formulations of the present invention are particularly advantageous because they permit absorption of a high concentration of the drug, and, following oral administration thereof, can permit rapid absorption of the drug into the bloodstream through inhibition of precipitation or crystallization of the drug and, thus, therapeutic concentrations can be achieved.

In one embodiment, the present invention comprises an orally deliverable liquid pharmaceutical composition, comprising (a) a bevirimat drug substance, and (b) a solvent liquid comprising (i) at least one pharmaceutically acceptable solvent, and (ii) a polymer, wherein the polymer is present in an amount sufficient to retard gelling and precipitation of the bevirimat in simulated gastric fluid over 48 hours at 37° C.

In one embodiment, the present invention provides an orally deliverable liquid pharmaceutical composition, comprising a bevirimat drug substance, and at least one pharmaceutically acceptable solvent. In one embodiment, the composition of the present invention further comprises a polymer. In one embodiment, the composition of the present invention further comprises a polymer in an amount sufficient to retard gelling and precipitation of bevirimat in simulated gastric fluid for about 24 hours at room temperature.

In one embodiment, the present invention provides an orally deliverable liquid pharmaceutical composition, comprising a bevirimat drug substance, preferably bevirimat-2NMG, propylene glycol, ethanol, and glycerin. The composition of the present invention can optionally contain one or more flavoring agents, and optionally a sweetener. Preferably, the weight/volume percent ratio of ethanol:propylene glycol:glycerin in the solvent liquid is about 1:about 2 to about 4:about 1. Preferably, the composition comprises from about 26 to about 38 w/v-% of propylene glycol, from about 10 to about 15 w/v-% of ethanol, and from about 10 to about 17 w/v-% of glycerin. More preferably, the composition comprises from about 27 to about 37 w/v-% of propylene glycol, from about 11 to about 14 w/v-% of ethanol, and from about 11 to about 16 w/v-% of glycerin. Advantageously, the composition comprises from about 32.6 to about 36 w/v-% of propylene glycol, from about 12.4 to about 13.6 w/v-% of ethanol, and from about 13.2 to about 14.6 w/v-% of glycerin.

In one embodiment, the present invention provides an orally deliverable liquid pharmaceutical composition, comprising a bevirimat drug substance present in an amount of from about 4 mg/mL to about 50 mg/mL free acid equivalents of bevirimat, preferably from about 7.5 mg/mL to about 25 mg/mL free acid equivalents of bevirimat, and more preferably from about 10 mg/mL to about 15 mg/mL free acid equivalents of bevirimat. In one embodiment, the present invention provides an orally deliverable liquid pharmaceutical composition, comprising a bevirimat drug substance present in an amount of from about 1 mg/mL to about 25 mg/mL free acid equivalents of bevirimat, preferably from about 3 mg/mL to about 18 mg/mL of bevirimat free acid equivalents, and more preferably from about 8 mg/mL to about 15 mg/mL of bevirimat free acid equivalents. In one embodiment, the present invention provides an orally deliverable liquid pharmaceutical composition, comprising a bevirimat drug substance present in an amount of about 12.5 mg/mL free acid equivalents of bevirimat. In this aspect of the invention, the orally deliverable liquid pharmaceutical composition of the present invention is provided as a unit dose. In another aspect of the invention, the orally deliverable liquid pharmaceutical composition of the present invention is provided in larger volumes, such as in a bulk bottle.

Suitable dosage volumes for the liquid pharmaceutical composition of the present invention are within the range of from about 4 mL to about 100 mL, preferably from about 8 mL to about 50 mL, more preferably from about 15 mL to about 35 mL, and advantageously from about 16 mL to about 32 mL for a composition having a concentration of about 12.5 mg/mL bevirimat free acid equivalents. In one embodiment, the dosage volume for a liquid pharmaceutical composition of the present invention having a concentration of 12.5 mg/mL bevirimat free acid equivalents is 32 mL.

In one embodiment, the present invention provides an orally deliverable liquid pharmaceutical composition comprising from about 100 mg to about 1000 mg of the bevirimat drug substance. The composition can be provided in one or more dose units each comprising from about 100 mg to about 1000 mg of the bevirimat drug substance.

In one embodiment, the present invention provides an orally deliverable liquid pharmaceutical composition, comprising from about 350 mg to about 600 mg of the bevirimat drug substance. In one embodiment, the present invention provides an orally deliverable liquid pharmaceutical composition, comprising from about 200 mg to about 600 mg of the bevirimat drug substance. In one embodiment, the present invention provides an orally deliverable liquid pharmaceutical composition, comprising from about 250 mg to about 450 mg of the bevirimat drug substance. In one embodiment, the present invention provides an orally deliverable liquid pharmaceutical composition, comprising from about 300 mg to about 400 mg of the bevirimat drug substance. In one embodiment, the present invention provides an orally deliverable liquid pharmaceutical composition, comprising about 350 mg of the bevirimat drug substance. These compositions can be provided in one or more dose units each comprising the described amount of the bevirimat drug substance.

In one embodiment, the present invention provides an orally deliverable liquid pharmaceutical composition, comprising from about 200 mg to about 500 mg of bevirimat free acid equivalents (corresponds to from about 330 to about 830 mg of bevirimat dimeglumine), and preferably from about 200 mg to about 300 mg of free acid equivalents (corresponds to from about 330 to about 498 mg of bevirimat dimeglumine). The compositions can be provided in one or more dose units each comprising the described amount of bevirimat free acid equivalents or bevirimat dimeglumine.

In one embodiment, the present invention provides an orally deliverable liquid pharmaceutical composition, comprising about 330 mg bevirimat dimeglumine, provided in one or more dose units each comprising about 330 mg of bevirimat dimeglumine.

In one embodiment, the present invention provides an orally deliverable liquid pharmaceutical composition, comprising a bevirimat drug substance and propylene glycol.

In one embodiment, the present invention provides an orally deliverable liquid pharmaceutical composition, comprising a bevirimat drug substance and ethanol.

In one embodiment, the present invention provides an orally deliverable liquid pharmaceutical composition, comprising a bevirimat drug substance and polyethylene glycol.

In one embodiment, the present invention provides an orally deliverable liquid pharmaceutical composition, comprising a bevirimat drug substance and glycerin.

In one embodiment, the present invention provides an orally deliverable liquid pharmaceutical composition, comprising a bevirimat drug substance and Vitamin E-TPGS.

In one embodiment, the present invention provides an orally deliverable liquid pharmaceutical composition, comprising a bevirimat drug substance present in an amount of 12.5 mg/mL bevirimat free acid equivalents; propylene glycol present in a 40% by volume relative to the final formulation; Tween80 present in a 0.25% by volume relative to the final formulation; Vitamin E-TPGS present in a 1% by volume relative to the final formulation, and glycerin present in a 20% by volume relative to the final formulation.

In one embodiment, the present invention provides an orally deliverable liquid pharmaceutical composition comprising a bevirimat drug substance present in an amount of 25 mg/mL bevirimat free acid equivalents; ethanol present in a 30% by volume relative to the final formulation, Vitamin E-TPGS present in a 1% by volume relative to the final formulation, and glycerin present in a 10% by volume relative to the final formulation.

In one aspect of the invention, the bevirimat drug substance is bevirimat dimeglumine.

In one aspect of the invention, the bevirimat drug substance is bevirimat dipotassium.

In one aspect of the invention, the bevirimat drug substance is bevirimat disodium.

Preferably, the orally deliverable liquid pharmaceutical composition comprises bevirimat-2NMG (bevirimat dimeglumine) as the bevirimat drug substance. Preferably, the orally deliverable liquid pharmaceutical composition comprises from about 0.01 to about 0.42 w/v-% of bevirimat-2NMG, more preferably from about 0.08 to about 0.35 w/v-% of bevirimat-2NMG, and advantageously from about 0.18 to about 0.25 w/v-% of bevirimat-2NMG.

Certain pharmaceutical compositions of the present invention exhibit one or more superior properties relative to unformulated bevirimat or other known compositions comprising bevirimat. These superior properties include, but are not limited to, one or more of the following:

(1) improved bioavailability;
(2) improved solubility of the pharmaceutical composition in gastric fluid;
(3) improved dispersion of bevirimat in gastric fluid;
(4) improved safety for oral dosage forms;
(5) improved chemical stability of the final oral dosage form;
(6) improved physical stability of the final oral dosage form;
(7) decreased conversion to metastable forms, including colloidal gels, of bevirimat; and
(8) decreased rate of precipitation in gastric fluid.

Generally, a poorly water-soluble drug can provide more rapid onset of therapeutic effect when orally administered as a liquid formulation relative to a solid dosage form because dissolution in the gastrointestinal tract is not required. Another advantage of a liquid formulation relative to a solid formulation is that disintegration of the dosage form is not required and drug is presented to gastrointestinal mucosa in a dissolved state suitable for immediate absorption.

Additionally, a drug administered in an imbibable solution can be available for absorption higher in the alimentary tract, for example, in the mouth and esophagus, than one that becomes available for absorption only upon disintegration of the carrier formulation in the stomach or bowel.

Another advantage of a liquid formulation for many subjects is that liquid dosage forms are easy to swallow. A yet further advantage of liquid formulations is that metering of doses is continuously variable, providing infinite dose flexibility. The benefits of ease of swallowing and dose flexibility are particularly advantageous for infants, children and the elderly. For these patients liquid dosage forms may result in increased patient compliance.

Some liquid formulations of the present invention are beneficial because such liquid formulations are less costly to package and easier to transport and handle than dilute solutions.

Other liquid formulations of the present invention are beneficial because such liquid formulations provide flexibility in administration and dosing as they can be administered with any desired degree of dilution. For example, a bevirimat liquid formulation could be provided at a specific dose levels to optimize absorption in a patient who is concomitantly taking other medicines that could inhibit or induce metabolism of the bevirimat liquid formulation. The flexibility inherent in a liquid formulation allows a physician to prescribe the optimal dose to each individual patient with respect to other medications that patient is taking.

Other liquid formulations of the present invention are beneficial because such liquid formulations that increase the ease of administration are more likely to increase patient compliance with the drug regimen relative to drug regimens consisting of anti-HIV pharmaceutical formulations exhibiting either or both a large pill size and a large number of pills/dose. Since lack of compliance with a drug regimen lead to sub-optimal blood plasma concentrations of a drug, which fosters the conditions for the faster emergence of drug resistant mutants, a formulation which increases patient compliance with a drug regimen, such as the liquid formulations described herein, is more likely to foster compliance and, in turn, support successful long term therapy. Other liquid formulations of the present invention are beneficial because such liquid formulations have a viscosity adjusted to impart, among other properties, facilitation of swallowing large or bulky solid oral dosage forms. As many oral HIV dosage forms are considered large or bulky, and as HIV drugs are usually administered in combination, the optimized viscosity of these liquid formulations can ease the swallowing burden associated with taking multiple large or bulky solid oral anti-HIV dosage forms.

Other liquid formulations of the present invention are beneficial because such liquid formulations comprise a bevirimat drug substance co-formulated with at least one other anti-HIV liquid dosage forms.

The pharmaceutical compositions of the present invention are useful where administration of a composition comprising an HIV-1 maturation inhibitor is indicated. It has been found that these compositions are particularly effective in the treatment of HIV-1 infection and AIDS.

It has been found that the pharmaceutical compositions of the present invention are able to solubilize bevirimat dimeglumine at desired concentrations. In some embodiments, the desired concentration is about 12.5 mg bevirimat free acid equivalents/mL.

It has been found that the pharmaceutical compositions of the present invention are able to provide an environment where bevirimat dimeglumine is stable in solution.

### Components

The active pharmaceutical ingredient employed in the present invention is bevirimat, which has the structure shown below:

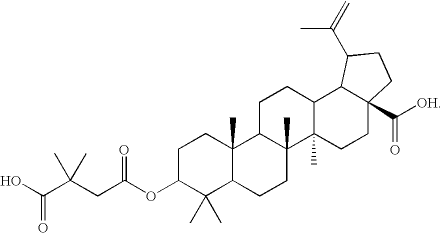

Even compared to other triterpene derivatives and other betulinic acid derivatives, bevirimat is a unique compound at least in part because it is a di-acid of high molecular weight. The presence of two carboxylic acid moieties contributes to localized dipole forces substantially different than those observed in mono-acid triterpene derivatives. Additionally, the steric hinderance of the C-28 carboxylic acid contributes to other charge interactions unique to this molecule. Bevirimat has low solubility in aqueous media and a high ClogP, which complicate its formulation. Additionally, it is believed that the presence of two carboxylic acid moieties contribute to the high fraction of protein binding (99+%) to human serum albumin which in turn results in a low free fraction of the bevirimat in the blood plasma. Additionally, bevirimat dimeglumine exhibits a strong tendency to form micelles in aqueous environments. By retarding dissolution, these micelles inhibit optimal absorption of bevirimat across the gastrointestinal membranes.

Bevirimat dimeglumine suitable for use in the formulations of the present invention exists in several solid-state forms, including an amorphous form and a plurality of morphic forms. Morphic forms include Form I and Form II. In some embodiments of the present invention, bevirimat dimeglumine used in accordance with the present invention can be amorphous, Form I, Form II, or a combination thereof.

In some embodiments of the present invention, the bevirimat drug substance consists of substantially phase pure bevirimat dimeglumine Form I.

In some embodiments of the present invention, the bevirimat drug substance comprises at least about 90% bevirimat dimeglumine Form I relative to all other solid state forms of bevirimat dimeglumine present in the bevirimat drug substance.

In some embodiments of the present invention, the bevirimat drug substance comprises at least about 75% bevirimat dimeglumine Form I relative to all other solid state forms of bevirimat dimeglumine present in the bevirimat drug substance.

In some embodiments of the present invention, the bevirimat drug substance comprises at least a detectable amount of bevirimat dimeglumine Form I relative to all other solid state forms of bevirimat dimeglumine present in the bevirimat drug substance.

In some embodiments of the present invention, the bevirimat drug substance consists of substantially phase pure bevirimat dimeglumine Form II.

In some embodiments of the present invention, the bevirimat drug substance comprises at least about 90% bevirimat dimeglumine Form II relative to all other solid state forms of bevirimat dimeglumine present in the bevirimat drug substance.

In some embodiments of the present invention, the bevirimat drug substance comprises at least about 75% bevirimat dimeglumine Form II relative to all other solid state forms of bevirimat dimeglumine present in the bevirimat drug substance.

In some embodiments of the present invention, the bevirimat drug substance comprises at least a detectable amount of bevirimat dimeglumine Form II relative to all other solid state forms of bevirimat dimeglumine present in the bevirimat drug substance.

In some embodiments of the present invention, the bevirimat drug substance consists of substantially phase pure amorphous bevirimat dimeglumine.

In some embodiments of the present invention, the bevirimat drug substance comprises at least about 90% amorphous bevirimat dimeglumine relative to all other solid state forms of bevirimat dimeglumine present in the bevirimat drug substance.

In some embodiments of the present invention, the bevirimat drug substance comprises at least about 75% amorphous bevirimat dimeglumine relative to all other solid state forms of bevirimat dimeglumine present in the bevirimat drug substance.

In some embodiments of the present invention, the bevirimat drug substance comprises at least a detectable amount of amorphous bevirimat dimeglumine relative to all other solid state forms of bevirimat dimeglumine present in the bevirimat drug substance.

In some embodiments of the present invention, bevirimat dimeglumine is present in an amount of from about 5 mg/mL to about 50 mg/mL bevirimat free acid equivalents. In some embodiments of the present invention, bevirimat dimeglumine is present in an amount of from about 7.5 mg/mL to about 25 mg/mL bevirimat free acid equivalents. In some embodiments of the present invention, bevirimat dimeglumine is present in an amount of from about 10 mg/mL to about 15 mg/mL bevirimat free acid equivalents. In some embodiments of the present invention, bevirimat dimeglumine is present in an amount of about 12.5 mg/mL bevirimat free acid equivalents.

Without wishing to be bound by theory, it appears that the bioavailability of orally deliverable liquid bevirimat dimeglumine formulations is inversely proportional to the concentration of bevirimat dimeglumine, present as bevirimat free acid equivalents, in a given formulation. As such, orally deliverable liquid bevirimat dimeglumine formulations comprising from about 10 mg/mL to about 15 mg/mL bevirimat free acid equivalents or about 12.5 mg/mL bevirimat free acid equivalents have been found to exhibit superior bioavailability profiles.

The present invention comprises at least one pharmaceutically acceptable solvent. Solvents suitable for use in the present invention include essentially any organic compound, any mixtures of organic compounds, or mixtures of one or more organic compounds and one or more aqueous compounds in which the combination of a bevirimat drug substance and the carrier materials are soluble and which exhibit an acceptable pharmacokinetic profile after oral administration to a patient. In one aspect, the pharmaceutically acceptable solvent comprises a polar solvent. Useful polar solvents include those having one or more hydroxyl moieties, especially one to six hydroxy moieties per solvent molecule. In one aspect, the pharmaceutically acceptable solvent has neutral or basic pH.

In some embodiments, the solvent liquid is selected from the group consisting of pharmaceutically acceptable glycols, glycerins, glycol ethers, alcohols, and combinations thereof.

In some embodiments, the pharmaceutically acceptable solvent comprises a solvent selected from the group consisting of alcohol, propylene glycol, glycerin, and combinations thereof; in some embodiments, the alcohol is ethanol. Preferably, at least one pharmaceutically acceptable solvent is propylene glycol. Useful pharmaceutically acceptable solvents include a combination of propylene glycol and glycerin.

Advantageously, the pharmaceutically acceptable solvent comprises a combination of ethanol, propylene glycol, and glycerin. Advantageously, ethanol, propylene glycol, and glycerin are present in the weight/volume percent ratio of about 1:from about 2 to about 4:about 1.

In some embodiments of the present invention, the solubilizer comprises a cyclodextrin. Illustrative cyclodextrins include α-cyclodextrins, β-cyclodextrins, and γ-cyclodextrins. Illustrative β-cyclodextrins include β-cyclodextrin and a water soluble derivatives of β-cyclodextrin such as sulfobutyl ether β-cyclodextrin, heptakis-2,6-di-O-methyl-β-cyclodextrin, hydroxypropyl-β-cyclodextrin, and dimethyl-β-cyclodextrin.

In some embodiments, the orally deliverable liquid pharmaceutical composition of the present invention does not comprise a polymer. Such embodiments employ a pharmaceutically acceptable solvent system and excipients that minimize gelling or precipitation.

In other embodiments of the present invention, the orally deliverable liquid pharmaceutical composition of the present invention comprises a polymer wherein the polymer is present in an amount sufficient to retard gelling and precipitation of the bevirimat in simulated gastric fluid. Polymers suitable for use in accordance with the present invention include natural, semi-synthetic, and synthetic polymers. Polymers suitable for use in accordance with the present invention include cellulosic polymers and non-cellulosic polymers.

In some embodiments, the polymer is a polyethylene glycol (“PEG”). In some embodiments, the PEG has an average molecular weight of about 100 to about 1,000. In some embodiments, the PEG has an average molecular weight of about 200 to about 600. In some embodiments, the PEG has an average molecular weight of about 375 to about 450. In some embodiments, the PEG has an average molecular weight of about 300 (“PEG 300”). In some embodiments, the PEG has an average molecular weight of about 400 (“PEG 400”). Advantageously, the polyethylene glycol is liquid.

In some embodiments, the polymer is a cellulosic polymer having at least a portion of substitutable hydroxyl groups substituted by either or both methoxyl and hydroxypropoxyl groups, in an amount effective to substantially inhibit crystallization or precipitation of the drug in simulated gastric fluid. Illustrative cellulosic polymers include hydroxypropyl celluloses, cellulose carboxymethyl ethers and their salts, hydroxypropyl methylcellulose phthalates, hydroxypropyl methylcellulose acetate succinate, hydroxypropylcelluloses (HPC), methylcelluloses (MC), carboxymethylcelluloses (CMC), hydroxypropylmethylcelluloses (HPMC), hydroxyethylcellulose (HEC) and hydroxyethylmethylcelluloses (HEMC). The cellulosic polymer(s) may be substituted at one or more positions so long as valencies remain appropriate; for example, carboxymethylcellulose is a cellulose derivative having an average of 1 to 3 hydroxy groups on each unit of the cellulose backbone substituted with a carboxymethyl group (—CH₂—COOH). Useful hydroxypropylmethylcelluloses include those having from about 15% to about 35% methoxyl substitution and from about 3% to about 15% hydroxypropoxyl substitution, preferably from about 19% to about 30% methoxyl substitution and from about 4% to about 12% hydroxypropoxyl substitution, and advantageously from about 19% to about 24% methoxyl substitution and from about 7% to about 12% hydroxypropoxyl substitution.

In some embodiments, the polymer comprises a non-cellulosic polymer. Illustrative non-cellulosic polymers include poly(methyl methacrylates), poly(vinyl alcohols), poly(vinyl pyrrolidones), poly(2-hydroxyethyl methacrylates), poly(N-vinyl pyrrolidones), poly(methyl methacrylates), poly(vinyl alcohols), poly(acrylic acids), poly(ethylene glycols), poly(methacrylic acids), poly(vinyl acetates), and propylene glycol alginates.

In some embodiments, the polymer is selected from the group consisting of polyvinylpyrrolidone and cellulosic polymers.

In some embodiments, the polymer comprises a polysorbate. In some embodiments the emulsifier is a polysorbate selected from the group consisting of Tween20, Tween40, Tween60, Tween80, Span20, Span40, Span60, Span80, and combinations thereof. In some embodiments the polysorbate is Tween80 or polyoxyethylene (20) sodium monooleate.

In some embodiments, the polymer comprises a poloxamer. Without wishing to be bound by theory, it is believed that in conjunction with bevirimat dimeglumine, poloxamers that typically act as nonionic surfactants also function as solubilizing agents and suspension stabilizers in the liquid dosage forms of the present invention. In some embodiments, poloxamer 188 is a preferred poloxamer. In some embodiments, poloxamer 407 is a preferred poloxamer. Poloxamer 188 is a nonionic block copolymer composed of 2 hydrophilic polyoxyethylene chains and connected by a hydrophobic polyoxypropylene chain.

In some embodiments, the polymer comprises a caprylocaproyl macrogolglyceride. Caprylocaproyl macrogolglycerides are mixtures of monoesters, diesters and triesters of glycerol and monoesters and diesters of polyethylene glycols with a mean molecular weight of about 200 to about 400. In some embodiments, the caprylocaproyl macrogolglyceride is PEG400 Caprylic/Capric Glyceride or Acconon MC-8 (CAS no. 91744-32-0, 223129-75-7).

In some embodiments, the polymer comprises a natural or synthetic gum containing galactomannans. Illustrative gums include including xanthan gums, tragacanths, acacias, agars, and guar gums.

Some embodiments of the invention employ a polymer that comprises a solubilizer. When present, the solubilizer is in amounts sufficient to solubilize a significant fraction of bevirimat drug substance in the liquid formulation and is capable of providing an immediate and therapeutically effective amount of bevirimat to a patient in a readily absorbable form upon administration. In some embodiments, the solubilizer of the present invention also increases the solubilization of bevirimat drug substance when the composition contacts an aqueous medium including gastro-intestinal fluids.

One type of solubilizer that may be used is a vitamin E substance. This group of solubilizers includes those belonging to the group of α-, β-, γ-, Δ-, ζ₁-, ζ₂- and η-tocopherols, their dl, d and 1 forms and their structural analogues, such as tocotrienols; the corresponding derivatives, e.g., esters, produced with organic acids; and mixtures thereof. As will be appreciated by those of skill in the art, only those vitamin E substances that effectively solubilize bevirimat may be included in the present compositions as solubilizers. Preferred vitamin E substance solubilizers include tocopherols, tocotrienols and tocopherol derivatives with organic acids such as acetic acid, propionic acid, bile acid, lactic acid, pyruvic acid, oxalic acid, malic acid, malonic acid, succinic acid, maleic acid, fumaric acid, tartaric acid, citric acid, benzoic acid, cinnamic acid, mandelic acid, polyethylene glycol succinate and salicylic acid.

Some embodiments of the present invention comprise Vitamin E alpha-tocopheryl polyethylene glycol succinate (“Vitamin E TPGS”) as sold by Eastman Chemicals. Vitamin E TPGS can be synthesized by esterifying d-alpha tocopheryl succinate with polyethylene glycol 1000 (“PEG 1000”). Particularly preferred vitamin E substances include alpha-tocopherol, alpha-tocopheryl acetate, alpha-tocopheryl acid succinate, alpha-tocopheryl polyethylene glycol 1000 succinate and mixtures thereof.

In some embodiments, the at least one pharmaceutically acceptable solvent is ethanol and the polymer is Vitamin E TPGS. This composition can further comprise Tween80, and optionally povidone. Povidone can be present, for example, in an amount of from about 0.2 to about 0.4 weight %.

Without wishing to be bound by theory, it is believed that Vitamin E TPGS enhances the absorption and bioavailability of bevirimat of the present invention as bevirimat has a large hydrophobic core the solubility of which can be improved in the presence of Vitamin E TPGS through micelle solubilization. As gastrointestinal malabsorption problems are often associated with HIV-1 disease progression in patients, the presence of an absorption enhancer is desirable. It is also believed that Vitamin E TPGS may increase the permeability of gastrointestinal membranes to large molecules such as bevirimat.

Although Vitamin E TPGS appears to have a beneficial effect on the absorption and bioavailability of bevirimat in the orally deliverable liquid pharmaceutical compositions of the present invention, it has been discovered that Vitamin E TPGS may lead to physical instability of some compositions over time during storage. The physical instability can be seen, for example, by visual inspection as gellation of the composition. It has now been discovered, that the physical stability of the liquid compositions of the present invention can be improved by reducing the amount of Vitamin E TPGS in the compositions of the present invention, or by completely excluding Vitamin E TPGS from the compositions.

It has been found that buffers, especially phosphate buffers, have beneficial effects on the physical stability of orally deliverable liquid pharmaceutical compositions of the present invention. Thus, incorporating phosphate buffer into pharmaceutical compositions of the present invention that include Vitamin E TPGS can balance the negative effects of Vitamin E TPGS on the stability of the compositions. Preferably, the orally deliverable liquid pharmaceutical composition of the present includes phosphate buffer and excludes Vitamin E TPGS.

In one aspect of the present invention, orally deliverable liquid pharmaceutical compositions useful in the present invention are those comprising a bevirimat drug substance, preferably bevirimat-2NMG, propylene glycol, ethanol, glycerin, and buffer providing a pH of from about 7.0 to about 9.0. The buffer is present in the composition in an amount effective to maintain a pH of from about 7.0 to about 9.0. In one embodiment, the final molarity of the buffer in the liquid pharmaceutical composition of the present invention is from about 0.01M to about 0.4M, preferably from about 0.05M to about 0.3M, and more preferably from about 0.1M to about 0.2M. Preferably, the pH of the buffer is pH 7.5-8.5, preferably pH 7.7-8.3, and more preferably pH 7.8-8.0. Suitable buffers include any buffers that do not react with the acid groups of bevirimat, and include, for example, phosphate buffers, citric acid-phosphate buffer, and imidazole-HCl buffer. Exemplary buffers suitable in the compositions of the present invention include those listed in Table 30 in Example 41. Preferably, the buffer is sodium phosphate buffer. Preferably, the composition comprises 0.2 M phosphate buffer, and more preferably the composition comprises 0.2 M sodium phosphate buffer, pH 7.9. Buffer, as used herein, refers to a buffering agent, either in a solid form or in an aqueous solution. The composition can optionally contain one or more flavoring agents, and optionally a sweetener.

Buffers that can react with the carboxylic acid groups of bevirimat, and therefore are not suitable in the liquid oral compositions of the present invention, include esters and primary amines, such as, for example, tromethamine (TRIS) buffer, glycine-HCl buffer, and glycyl-glycine buffer.

In one embodiment, orally deliverable liquid pharmaceutical compositions useful in the present invention are those comprising a bevirimat drug substance, preferably bevirimat-2NMG, propylene glycol, ethanol, glycerin and phosphate buffer. Preferably, the phosphate buffer is 0.1-0.2M sodium phosphate buffer, pH 7.8-7.9. Suitable phosphate buffers include sodium phosphate buffer and potassium phosphate buffer. Suitable concentrations of the phosphate buffer include from about 0.050M to about 0.375M, preferably from about 0.75M to about 0.3M, and more preferably from about 0.1M to about 0.2M. The pH of the phosphate buffer can be pH 7.5-8.5, preferably pH 7.7-8.3, and more preferably pH 7.8-8.0. Preferably, the composition comprises from 20 to about 31 w/v-% of 0.2 M phosphate buffer, pH 7.9; more preferably from about 22 to about 30 w/v-% of 0.2 M phosphate buffer, pH 7.9, and advantageously from about 26.2 to about 29 w/v-% of phosphate buffer, pH 7.9. Suitable molar ratios of phosphate:bevirimat free di-acid include from about 1.25:1 to about 5:1, and more preferably about 2.5:1. Sodium phosphate and potassium phosphate buffers can be prepared by methods know in the art or they are corrercially available from, for example, VWR Scientific and Fisher Scientific. Sodium phosphate buffer is typically made by preparing equimolar solutions of sodium phosphate monobasic and sodium phosphate dibasic and mixing the solutions to achieve the final pH. Blending tables are readily available in the art.

In one embodiment, the liquid pharmaceutical composition of the present invention further comprises one or more flavoring agents, and optionally a sweetener.

Some flavoring agents can contribute to the chemical instability of the orally deliverable liquid pharmaceutical compositions of the present invention, whereas some flavoring agents do not negatively affect the chemical stability. It has been found that flavoring agents, such as mint, menthol, peach, Dulce de Leche, coffee, orange, orange/vanilla, strawberry, peach/mango, punch, chocolate, mixed berry, mocha, strawberry/banana, and banana do not negatively affect the chemical stability. Accordingly, one aspect of the present invention provides an orally deliverable liquid pharmaceutical composition, comprising a bevirimat drug substance, preferably bevirimat-2NMG, propylene glycol, ethanol, glycerin, buffer providing a pH of from about 7.0 to about 9.0 (preferably phosphate buffer), and one or more flavoring agents selected from the group consisting of mint, menthol, peach, Dulce de Leche, coffee, orange, orange/vanilla, strawberry, peach/mango, punch, chocolate, mixed berry, mocha, strawberry/banana, and banana. Preferably, the one or more flavoring agents comprise mint, menthol, or combinations thereof. Useful compositions of the present invention include those, wherein the one or more flavoring agents are present in a total amount of from about 0.1 to about 1.5 w/v-%, preferably from about 0.2 to about 1.3 w/v-%, and more preferably from about 0.25 to about 1.2 w/v-%. Preferably, the orally deliverable liquid pharmaceutical composition comprises from about 0.4 to about 0.7 w/v-% menthol or from about 0.1 to about 0.8 w/v-% of mint; more preferably from about 0.45 to about 0.65 w/v-% of menthol or from about 0.2 to about 0.6 w/v-% of mint; and advantageously from about 0.53 to about 0.60 w/v-% of menthol or from about 0.25 to about 0.50 w/v-% of mint.

In some embodiments the solubilizer comprises a monohydric alcohol ester of an organic acid. Illustrative monohydric alcohols include ethanol, isopropanol, t-butanol, a fatty alcohol, phenol, cresol, benzyl alcohol or a cycloalkyl alcohol. Illustrative organic acids include acetic acid, propionic acid, butyric acid, a fatty acid of 6-22 carbon atoms, lactic acid, pyruvic acid, malic acid, malonic acid, tartaric acid, citric acid, benzoic acid, cinnamic acid. When the present invention comprises an acid, it is generally preferred that the acid is present as a component of a buffer. Illustrative solubilizers in this group include trialkyl citrates, lower alcohol fatty acid esters and lactones. Illustrative trialkyl citrates include triethyl citrate, acetyltriethyl citrate, tributyl citrate, acetyltributyl citrate and mixtures thereof with triethyl citrate being particularly preferred. Illustrative lower alcohol fatty acid esters include ethyl oleate, ethyl linoleate, ethyl caprylate, ethyl caprate, isopropyl myristate, isopropyl palmitate and combinations thereof.

In some embodiments the solubilizer comprises a lactone. Illustrative examples include ε-caprolactone, Δ-valerolactone, β-butyrolactone, and combinations thereof.

In some embodiments, the solubilizer is a nitrogen-containing solvent. Illustrative nitrogen-containing solvents include dimethylformamide, dimethylacetamide, N-alkylpyrrolidone, N-hydroxyalkylpyrrolidone, N-alkylpiperidone, N-alkylcaprolactam and mixtures thereof wherein alkyl is a C₁₋₁₂ branched or straight chain alkyl. Particularly preferred nitrogen-containing solvents include N-methyl-2-pyrrolidone, N-ethyl-2-pyrrolidone or combinations thereof. Alternatively, the nitrogen-containing solvent may be in the form of a polymer such as polyvinylpyrrolidone.

In some embodiments the solubilizer comprises a glycerol acetate. Illustrative glycerol acetates include acetin, diacetin, triacetin and combinations thereof.

In some embodiments the solubilizer comprises an acetylated glycerol fatty acid ester. Illustrative acetylated glycerol fatty acid esters include acetylated monoglycerides, acetylated diglycerides and combinations thereof. In one embodiment, the acetylated monoglyceride is a distilled acetylated monoglyceride.

In some embodiments the solubilizer comprises a polyoxyethylene castor oil derivative. Polyoxyethylene derivatives are complex mixtures of various hydrophobic and hydrophilic components. Members with each range have different degrees of ethoxylation (moles/PEG unit)s as indicated in their numerical suffix (n). The chemical structures of the polyethoxylated hydrogenated castor oils are analogous to polyethoxylated castor oils with the exception that the double bond in the fatty chain has been saturated by hydrogenation. The European Pharmacopoiea 2005 (PhEur 2005) states that polyoxyl castor oil contains mainly ricinoleyl glycerol ethoxylated with 30-50 molecules of ethylene oxide (nominal value), with small amounts of macrogol ricinoleate and of the corresponding free glycols. The PhEur 2005 also states that polyoxyl hydrogenated castor oil contains mainly trihydroxystearyl glycerol ethoxylated with 7-60 molecules of ethylene oxide (nominal value). Illustrative examples include polyoxyl 5 castor oil (Acconon™ CA-5); Polyoxyl 9 castor oil (Acconon™ CA-9); Polyoxyl 35 castor oil (Cremophor™ EL); and Polyoxyl 40 hydrogenated castor oil (Cremophor™ RH 40). The solubilizer may be a propylene glycol ester. Illustrative propylene glycol esters include propylene carbonate, propylene glycol monoacetate, propylene glycol diacetate, propylene glycol fatty acid esters, acetylated propylene glycol fatty acid esters and mixtures thereof. Alternatively, the propylene glycol fatty acid ester may be a propylene glycol fatty acid monoester, propylene glycol fatty acid diester or mixture thereof. In some embodiments the propylene glycol ester is propylene glycol monocaprylate. Other illustrative propylene glycol esters include propylene glycol dicaprylate, and combinations thereof.

Another group of solubilizers are ethylene glycol esters. Illustrative ethylene glycol esters include monoethylene glycol monoacetates, diethylene glycol esters, polyethylene glycol esters and combinations thereof. Additional illustrative examples include ethylene glycol monoacetates, ethylene glycol diacetates, ethylene glycol fatty acid monoesters, ethylene glycol fatty acid diesters, and combinations thereof. Alternatively, the ethylene glycol ester may be a polyethylene glycol fatty acid monoesters, polyethylene glycol fatty acid diesters or combinations thereof. In some embodiments, the ethylene glycol esters are those obtained from the transesterification of polyethylene glycol with a triglyceride or combinations thereof.

In some embodiments, the polymer comprises a polyvinyl pyrrolidone (also known as PVP or povidone). In some embodiments, the PVP is PVPK15. In some embodiments, the PVP is PVP K30. In some embodiments, the PVP is PVP K29. In some embodiments, the PVP is PVP K90. In some embodiments, the PVP is Povidone K11. In some embodiments, the PVP is Povidone K17.

Some embodiments of the present invention comprise a polymer present in an amount sufficient to retard gelling and precipitation of the bevirimat upon introduction of the formulation into stirred simulated gastric fluid (0.2% NaCl, 0.32% pepsin, 0.1N HCl, pH 1.2) over 48 hours at 37° C. In some embodiments, the polymer is present in an amount sufficient to retard gelling and precipitation of the bevirimat upon introduction of the formulation into stirred simulated gastric fluid over 72 hours at 37° C. In some embodiments, the polymer is present in an amount sufficient to retard gelling and precipitation of the bevirimat upon introduction of the formulation into stirred simulated gastric fluid over 96 hours at 37° C. In some embodiments, the polymer is present in an amount sufficient to retard gelling and precipitation of the bevirimat upon introduction of the formulation into stirred simulated gastric fluid over 48 hours at room temperature. In some embodiments, the polymer is present in an amount sufficient to retard gelling and precipitation of the bevirimat upon introduction of the formulation into stirred simulated gastric fluid over 72 hours at room temperature. In some embodiments, the polymer is present in an amount sufficient to retard gelling and precipitation of the bevirimat upon introduction of the formulation into stirred simulated gastric fluid over 96 hours at room temperature.

Some embodiments of the present invention further comprise one or more pharmaceutically-acceptable excipients selected from the group consisting of absorbents, acids, adjuvants, antifoamers, anticoagulants, antimicrobials, antifungals, antioxidants, antiphlogistics, astringents, antiseptics, bases, binders, chelating agents, sequestrants, coagulants, coating agents, colorants, dyes, pigments, compatibilizers, complexing agents, softeners, crystal growth inhibitors, denaturants, dehydrating agents, diluents, dispersants, emollients, emulsifiers, enzymes, extenders, flavor masking agents, flavorants, fragrances, gelling, humectants, moisturizers, buffers, pH control agents, preservatives, soothing agents, sweeteners, retarding agents, stabilizers, suspending agents, sweeteners, thickening agents, consistency regulators, surfactants, polymers, preservatives, antigellants, rheology control agents, UV absorbers, and viscosity modulators. One or more additives from any particular class, as well as one or more different classes of additives, may be present in the compositions.

Some embodiments of the present invention further comprise a coloring agent. Illustrative coloring agents include, either individually or in combination, FD&C Red No. 3 and FD&C Blue No. 1.

Some embodiments of the present invention further comprise a sweetener. Illustrative sweeteners include sugars and sugar alcohols, either individually or in combination: sucrose, glucose, fructose, high fructose corn syrup, saccharin, sorbitol, xylitol, erythritol, sucralose, maltodextrin malt extract syrup, mannitol, Acesulfame potassium, mono-ammonium glycyrrhizinate (MAG or Magnasweet®) and aspartame. In some embodiments sweeteners such as sucrose, glucose, saccharin and sorbitol are present from about 0.1% to about 10% weight percent. In some embodiments, xylitol is used as a sweetener. Useful orally deliverable liquid pharmaceutical compositions include those comprising from about 4 to about 7 w/v-% of xylitol, preferably from about 4.5 to about 6.5 w/v-% of xylitol, and more preferably from about 5.25 to about 6.0 w/v-% of xylitol.

Liquid oral compositions of the present invention comprising ethanol, propylene glycol and glycerin can prevent microbial growth and, therefore, can be considered a self-preserving composition. In this aspect of the invention, some embodiments of the present invention do not comprise a preservative.

Some embodiments of the present invention further comprise a preservative. In some embodiments, a preservative is employed to prevent microbial growth. Illustrative antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol and thimerosal. In some embodiments, a preservative is employed to prevent fungal growth. Illustrative antifungal preservations include butyl paraben, methyl paraben, ethyl paraben, propyl paraben, benzoic acid, potassium sorbate, sodium benzoate, sodium propionate and sorbic acid.

Some embodiments of the present invention further comprise a flavoring agent. Flavoring agents include essential oils, synthetic flavors or mixtures thereof. Artificial flavoring agents can also be used. Natural and artificial flavoring agents can be combined in any sensorially acceptable fashion. Flavoring agents are commercially available from, for example, Virginia Dare (882 Third Ave., Brooklyn, N.Y.), FONA International Inc. (1900 Averill Road Geneva, Ill. 60134), and David Michael & Co., Inc. (10801 Decatur Road, Philadelphia, Pa. 19154). Flavoring agents suitable for use in the pharmaceutical compositions of present invention include those that do not negatively affect the chemical stability of the liquid pharmaceutical composition. Suitable exemplary flavoring agents are listed in Tables 31 and 32 in Example 42. Tables 31 and 32 also provide information on the supplier, the catalog number, and the starting usage or upper usage level of each flavoring agent. Especially suitable flavoring agents include, either individually or in combination, mint, menthol (including L-menthol), peach, Dulce de Leche, coffee, peach, orange, orange/vanilla, strawberry, peach/mango, punch, chocolate, mixed berry, mocha, strawberry/banana, and banana, and preferably mint and menthol. The flavoring agent is present in an amount effective to provide the desirable taste characterictics. In some embodiments flavoring agents are present from about 0.01% to about 10% weight percent. Suitable concentrations of the one or more flavoring agents in the liquid pharmaceutical compositions of the present invention will be apparent to a person of ordinary skill based on the suppliers' recommendations. Useful compositions of the invention include those where one or more flavoring agents are present in a total amount of from about 0.1 to about 1.5 w/v-%, preferably from about 0.2 to about 1.3 w/v-%, and more preferably from about 0.25 to about 1.2 w/v-%. The concentration w/v-% refers to the weight (in grams) of the commercially available flavoring agent for every 100 mL of the liquid pharmaceutical composition of the present invention. Examples of flavoring agents that may have a negative effect on the chemical stability of the pharmaceutical compositions of the present invention are those that have high contents of reactive ester groups.

Some embodiments of the present invention further comprise a pH modifier or buffer. Illustrative pH modifiers or buffers include, either individually or in combination, carboxylic acids such as citric acid, ascorbic acid, lactic acid, and succinic acid.

Some embodiments of the present invention further comprise a gastrointestinal permeation enhancer. Illustrative enhancers include, either individually or in combination, alkali metal salts of salicyclic acid, caprylic and capric acid, such as sodium salicylate, sodium caprylate and sodium caprate.

### Dosage Strengths and Dose Number

Dosage strengths of the pharmaceutical compositions can typically contain, for example, 100, 150, 200, 250, 300, 350, 400, 450, 500, 550, 600, 650, 700, 750, 800, 850, 900, 950, or 1,000 mg of the bevirimat drug substance. Some dosage strengths contain about 250, 300, 350, 400, 450, or 500 mg of the bevirimat drug substance. Advantageously, some dosage strengths contain 200 mg or 300 mg of the bevirimat drug substance.

In one aspect of the present invention, suitable dosage strengths contain from about 350 to about 600 mg of the bevirimat drug substance. Some dosage strengths contain from about 350 to about 400 mg of the bevirimat drug substance. Some dosage strengths contain from about 400 to about 600 mg of the bevirimat drug substance.

In one aspect of the invention, suitable oral unit dosage forms of the present invention include those having from about 200 mg to about 500 mg of bevirimat free acid equivalents (corresponds to from about 330 mg to about 830 mg of bevirimat dimeglumine), and preferably from about 200 to about 300 mg of bevirimat free acid equivalents (corresponds to from about 330 mg to about 498 mg of bevirimat dimeglumine).

In one aspect of the invention, suitable oral unit dosage forms include those having from about 355 mg to about 475 mg of bevirimat dimeglumine, and advantageously about 330 mg of bevirimat dimeglumine.

The dosage strength can be selected to accommodate the desired frequency of administration used to achieve the specified daily dosage. The dosage regimen of the pharmaceutical composition that is administered depends on a variety of factors, including the age, weight, sex and medical condition of the subject, and thus can vary widely.

It has been discovered, however, that the dosage strengths of the present invention are suitable for once-a-day or twice-a-day administration. Once-a-day administration of such bevirimat formulations is preferred for convenience of administration and to facilitate increased of patient compliance.

### Specific Embodiments

Illustrative embodiments of single dose liquid formulations of the present invention are described below. Unless otherwise indicated, all percentages refer to weight percent.

In one embodiment, a single dose liquid formulation of the present invention comprises about 250 mg bevirimat dimeglumine, about 1.89 g ethanol, about 1.87 g glycerin, about 4.97 g propylene glycol, about 0.16 g Vitamin E TPGS, with sterilized water for irrigation q.s. to 16 mL.

In one embodiment, a single dose liquid formulation of the present invention comprises about 300 mg bevirimat dimeglumine, about 1.89 g ethanol, about 1.87 g glycerin, about 4.97 g propylene glycol, about 0.16 g Vitamin E TPGS, with sterilized water for irrigation q.s. to 16 mL.

In one embodiment, a single dose liquid formulation of the present invention comprises about 350 mg bevirimat dimeglumine, about 1.89 g ethanol, about 1.87 g glycerin, about 4.97 g propylene glycol, about 0.16 g Vitamin E TPGS, with sterilized water for irrigation q.s. to 16 mL.

In one embodiment, a single dose liquid formulation of the present invention comprises about 400 mg bevirimat dimeglumine, about 1.89 g ethanol, about 1.87 g glycerin, about 4.97 g propylene glycol, about 0.16 g Vitamin E TPGS, with sterilized water for irrigation q.s. to 16 mL.

In one embodiment, a single dose liquid formulation of the present invention comprises about 450 mg bevirimat dimeglumine, about 2.27 g ethanol, about 1.87 g glycerin, about 4.97 g propylene glycol, about 0.16 g Vitamin E TPGS, with sterilized water for irrigation q.s. to 16 mL.

In one embodiment, a single dose liquid formulation of the present invention comprises about 350 mg bevirimat dimeglumine, about 1.65 g ethanol, about 1.87 g glycerin, about 4.97 g propylene glycol, about 0.16 g Vitamin E TPGS, with sterilized water for irrigation q.s. to 16 mL.

In one embodiment, a single dose liquid formulation of the present invention comprises about 350 mg bevirimat dimeglumine, about 1.90 g ethanol, about 2.26 g glycerin, about 4.97 g propylene glycol, about 0.16 g Vitamin E TPGS, with sterilized water for irrigation q.s. to 16 mL.

In one embodiment, a single dose liquid formulation of the present invention comprises about 350 mg bevirimat dimeglumine, about 1.90 g ethanol, about 1.58 g glycerin, about 4.97 g propylene glycol, about 0.16 g Vitamin E TPGS, with sterilized water for irrigation q.s. to 16 mL.

In one embodiment, a single dose liquid formulation of the present invention comprises about 350 mg bevirimat dimeglumine, about 1.90 g ethanol, about 1.90 g glycerin, about 5.51 g propylene glycol, about 0.16 g Vitamin E TPGS, with sterilized water for irrigation q.s. to 16 mL.

In one embodiment, a single dose liquid formulation of the present invention comprises about 350 mg bevirimat dimeglumine, about 1.90 g ethanol, about 1.87 g glycerin, about 4.42 g propylene glycol, about 0.16 g Vitamin E TPGS, with sterilized water for irrigation q.s. to 16 mL.

In one embodiment, a single dose liquid formulation of the present invention comprises about 400 mg bevirimat dimeglumine, about 1.89 g ethanol, about 1.87 g glycerin, about 4.97 g propylene glycol, about 0.10 g Vitamin E TPGS, with sterilized water for irrigation q.s. to 16 mL.

In one embodiment, a single dose liquid formulation of the present invention comprises about 400 mg bevirimat dimeglumine, about 1.89 g ethanol, about 1.87 g glycerin, about 4.97 g propylene glycol, about 0.20 g Vitamin E TPGS, with sterilized water for irrigation q.s. to 16 mL.

In one embodiment, a single dose liquid formulation of the present invention comprises about 450 mg bevirimat dimeglumine, about 1.89 g ethanol, about 1.87 g glycerin, about 4.97 g propylene glycol, about 0.25 g Vitamin E TPGS, with sterilized water for irrigation q.s. to 16 mL.

In one embodiment, a single dose liquid formulation of the present invention comprises from about 300 mg to about 400 mg bevirimat free acid equivalents and a combination of carrier materials comprising ethanol, propylene glycol, and glycerin wherein the weight/volume percent ratio of ethanol:propylene glycol:glycerin is from about 2 to about 4: from about 2 to about 4:about 1, and wherein the aggregate amount of the single dose liquid formulation is from about 8 g to about 11 g.

In one embodiment, a single dose liquid formulation of the present invention comprises from about 325 mg to about 475 mg bevirimat free acid equivalents and a combination of carrier materials comprising ethanol, propylene glycol, and glycerin wherein the weight/volume percent ratio of ethanol:propylene glycol:glycerin is from about 2 to about 4: from about 2 to about 4:about 1, and wherein the aggregate amount of the single dose liquid formulation is from about 9 g to about 10 g.

In one embodiment, a single dose liquid formulation of the present invention comprises about 350 mg bevirimat free acid equivalents and a combination of carrier materials comprising ethanol, propylene glycol, and glycerin wherein the weight/volume percent ratio of ethanol:propylene glycol:glycerin is from about 2 to about 4: from about 2 to about 4:about 1, and wherein the aggregate amount of the single dose liquid formulation is about 9.25 g.

In one embodiment, a single dose liquid formulation of the present invention comprises from about 300 mg to about 400 mg bevirimat free acid equivalents and a combination of carrier materials comprising ethanol, propylene glycol, and glycerin wherein the weight/volume percent ratio of ethanol:propylene glycol:glycerin is about 3:about 3:about 1, and wherein the aggregate amount of the single dose liquid formulation is from about 8 g to about 11 g.

In one embodiment, a single dose liquid formulation of the present invention comprises from about 325 mg to about 475 mg bevirimat free acid equivalents and a combination of carrier materials comprising ethanol, propylene glycol, and glycerin wherein the weight/volume percent ratio of ethanol:propylene glycol:glycerin is about 3:about 3:about 1, and wherein the aggregate amount of the single dose liquid formulation is from about 9 g to about 10 g.

In one embodiment, a single dose liquid formulation of the present invention comprises about 350 mg bevirimat free acid equivalents and a combination of carrier materials comprising ethanol, propylene glycol, and glycerin wherein the weight/volume percent ratio of ethanol:propylene glycol:glycerin is from about 2 to about 3:about 3:about 1, and wherein the aggregate amount of the single dose liquid formulation is about 9.25 g.

In one embodiment, a single dose liquid formulation of the present invention comprises from about 300 mg to about 400 mg bevirimat free acid equivalents and a combination of carrier materials comprising ethanol, propylene glycol, and glycerin wherein the weight/volume percent ratio of ethanol:propylene glycol:glycerin is about 2:about 2:about 1, and wherein the aggregate amount of the single dose liquid formulation is from about 8 g to about 11 g.

In one embodiment, a single dose liquid formulation of the present invention comprises from about 325 mg to about 475 mg bevirimat free acid equivalents and a combination of carrier materials comprising ethanol, propylene glycol, and glycerin wherein the weight/volume percent ratio of ethanol:propylene glycol:glycerin is about 2:about 2:about 1, and wherein the aggregate amount of the single dose liquid formulation is from about 9 g to about 10 g.

In one embodiment, a single dose liquid formulation of the present invention comprises about 350 mg bevirimat free acid equivalents and a combination of carrier materials comprising ethanol, propylene glycol, and glycerin wherein the weight/volume percent ratio of ethanol:propylene glycol:glycerin is about 2:about 2:about 1, and wherein the aggregate amount of the single dose liquid formulation is about 9.25 g.

In one embodiment, a single dose liquid formulation of the present invention comprises about 250 mg bevirimat dimeglumine, about 3.74 g glycerin, about 6.63 g propylene glycol, about 0.16 g Vitamin E TPGS, 0.032 g povidone K15, with sterilized water for irrigation q.s. to 16 mL.

In one embodiment, a single dose liquid formulation of the present invention comprises about 300 mg bevirimat dimeglumine, about 3.74 g glycerin, about 6.63 g propylene glycol, about 0.16 g Vitamin E TPGS, 0.032 g povidone K15, with sterilized water for irrigation q.s. to 16 mL.

In one embodiment, a single dose liquid formulation of the present invention comprises about 350 mg bevirimat dimeglumine, about 3.74 g glycerin, about 6.63 g propylene glycol, about 0.16 g Vitamin E TPGS, 0.032 g povidone K15, with sterilized water for irrigation q.s. to 16 mL.

In one embodiment, a single dose liquid formulation of the present invention comprises about 400 mg bevirimat dimeglumine, about 3.74 g glycerin, about 6.63 g propylene glycol, about 0.16 g Vitamin E TPGS, 0.032 g povidone K15, with sterilized water for irrigation q.s. to 16 mL.

In one embodiment, a single dose liquid formulation of the present invention comprises about 350 mg bevirimat dimeglumine, about 4.10 g glycerin, about 6.63 g propylene glycol, about 0.16 g Vitamin E TPGS, 0.032 g povidone K15, with sterilized water for irrigation q.s. to 16 mL.

In one embodiment, a single dose liquid formulation of the present invention comprises about 350 mg bevirimat dimeglumine, about 3.37 g glycerin, about 6.63 g propylene glycol, about 0.16 g Vitamin E TPGS, 0.032 g povidone K15, with sterilized water for irrigation q.s. to 16 mL.

In one embodiment, a single dose liquid formulation of the present invention comprises about 350 mg bevirimat dimeglumine, about 3.75 g glycerin, about 7.25 g propylene glycol, about 0.16 g Vitamin E TPGS, 0.032 g povidone K15, with sterilized water for irrigation q.s. to 16 mL.

In one embodiment, a single dose liquid formulation of the present invention comprises about 350 mg bevirimat dimeglumine, about 3.74 g glycerin, about 5.90 g propylene glycol, about 0.16 g Vitamin E TPGS, 0.032 g povidone K15, with sterilized water for irrigation q.s. to 16 mL.

In one embodiment, a single dose liquid formulation of the present invention comprises about 350 mg bevirimat dimeglumine, about 3.75 g glycerin, about 6.63 g propylene glycol, about 0.20 g Vitamin E TPGS, 0.032 g povidone K15, with sterilized water for irrigation q.s. to 16 mL.

In one embodiment, a single dose liquid formulation of the present invention comprises about 400 mg bevirimat dimeglumine, about 3.74 g glycerin, about 6.63 g propylene glycol, about 0.24 g Vitamin E TPGS, 0.032 g povidone K15, with sterilized water for irrigation q.s. to 16 mL.

In one embodiment, a single dose liquid formulation of the present invention comprises about 350 mg bevirimat dimeglumine, about 3.74 g glycerin, about 6.63 g propylene glycol, about 0.16 g Vitamin E TPGS, 0.038 g povidone K15, with sterilized water for irrigation q.s. to 16 mL.

In one embodiment, a single dose liquid formulation of the present invention comprises about 350 mg bevirimat dimeglumine, about 3.74 g glycerin, about 6.63 g propylene glycol, about 0.16 g Vitamin E TPGS, 0.024 g povidone K15, with sterilized water for irrigation q.s. to 16 mL.

In one embodiment, a single dose liquid formulation of the present invention comprises from about 300 mg to about 400 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 5:about 1, and wherein the aggregate amount of the single dose liquid formulation is from about 9 g to about 12 g.

In one embodiment, a single dose liquid formulation of the present invention comprises from about 325 mg to about 375 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 5:about 1, and wherein the aggregate amount of the single dose liquid formulation is from about 10.5 g to about 11.5 g.

In one embodiment, a single dose liquid formulation of the present invention comprises from about 350 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 5:about 1, and wherein the aggregate amount of the single dose liquid formulation is about 11.0 g.

In one embodiment, a single dose liquid formulation of the present invention comprises from about 300 mg to about 400 mg bevirimat free acid equivalents and a combination of carrier materials comprising propylene glycol, glycerin, Vitamin E TPGS, and povidone K15 wherein the weight/volume percent ratio of propylene glycol:glycerin: Vitamin E TPGS:povidone K15 is about 200:about 135:about 5:about 1, and wherein the aggregate amount of the single dose liquid formulation is from about 9 g to about 12 g.

In one embodiment, a single dose liquid formulation of the present invention comprises from about 325 mg to about 375 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 135:about 5:about 1, and wherein the aggregate amount of the single dose liquid formulation is from about 10.5 g to about 11.5 g.

In one embodiment, a single dose liquid formulation of the present invention comprises from about 350 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 135:about 5:about 1, and wherein the aggregate amount of the single dose liquid formulation is about 11.0 g.

In one embodiment, a single dose liquid formulation of the present invention comprises from about 300 mg to about 400 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 250:about 120:about 5:about 1, and wherein the aggregate amount of the single dose liquid formulation is from about 9 g to about 12 g.

In one embodiment, a single dose liquid formulation of the present invention comprises from about 325 mg to about 375 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 250:about 120:about 5:about 1, and wherein the aggregate amount of the single dose liquid formulation is from about 10.5 g to about 11.5 g.

In one embodiment, a single dose liquid formulation of the present invention comprises about 350 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 250:about 120:about 5:about 1, and wherein the aggregate amount of the single dose liquid formulation is about 11.0 g.

In one embodiment, a single dose liquid formulation of the present invention comprises from about 300 mg to about 400 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 7.5:about 1, and wherein the aggregate amount of the single dose liquid formulation is from about 9 g to about 12 g.

In one embodiment, a single dose liquid formulation of the present invention comprises from about 325 mg to about 375 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 7.5:about 1, and wherein the aggregate amount of the single dose liquid formulation is from about 10.5 g to about 11.5 g.

In one embodiment, a single dose liquid formulation of the present invention comprises about 350 mg bevirimat and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 7.5:about 1, and wherein the aggregate amount of the single dose liquid formulation is about 11.0 g.

In one embodiment, a single dose liquid formulation of the present invention comprises from about 300 mg to about 400 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 5:about 1.5, and wherein the aggregate amount of the single dose liquid formulation is from about 9 g to about 12 g.

In one embodiment, a single dose liquid formulation of the present invention comprises from about 325 mg to about 375 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 120:about 5:about 1.5, and wherein the aggregate amount of the single dose liquid formulation is from about 10.5 g to about 11.5 g.

In one embodiment, a single dose liquid formulation of the present invention comprises about 350 mg bevirimat free acid equivalents and a combination of carrier materials comprising propylene glycol, glycerin, Vitamin E TPGS, and povidone K15 wherein the weight/volume percent ratio of propylene glycol:glycerin:Vitamin E TPGS:povidone K15 is about 200:about 60:about 5:about 1.5, and wherein the aggregate amount of the single dose liquid formulation is about 11.0 g.

In still another embodiment, the pharmaceutical compositions of the present invention comprise a bevirimat drug substance and one or more carrier materials in the form of an oral unit dosage suitable for once-a-day oral administration.

In one aspect of the present invention, the liquid formulation composition of the present invention includes buffer (preferably phosphate buffer) and excludes Vitamin E TPGS. Accordingly, in one embodiment, a single dose liquid formulation of the present invention comprises about 330 mg of bevirimat dimeglumine, from about 1.90 g to about 2.30 g of ethanol, from about 2.0 g to about 2.50 g of glycerin, from about 4.90 to about 6.0 g of propylene glycol, and from about 4.00 g to about 4.80 g of 0.2 M sodium phosphate buffer, pH 7.9.

In one embodiment, a single dose liquid formulation of the present invention comprises about 330 mg bevirimat dimeglumine, about 1.90 g ethanol, about 2.02 g glycerin, about 4.99 g propylene glycol and 4.00 g of 0.2 M sodium phosphate buffer, pH 7.9, with sterilized water q.s. to 16 mL.

In one embodiment, a single dose liquid formulation of the present invention comprises about 330 mg bevirimat dimeglumine, about 1.98 g ethanol, about 2.12 g glycerin, about 5.22 g propylene glycol and 4.20 g of 0.2 M sodium phosphate buffer, pH 7.9, with sterilized water q.s. to 16 mL.

In one embodiment, a single dose liquid formulation of the present invention comprises about 330 mg bevirimat dimeglumine, about 2.07 g ethanol, about 2.22 g glycerin, about 5.47 g propylene glycol and 4.40 g of 0.2 M sodium phosphate buffer, pH 7.9, with sterilized water q.s. to 16 mL.

In one embodiment, a single dose liquid formulation of the present invention comprises about 330 mg bevirimat dimeglumine, about 2.17 g ethanol, about 2.32 g glycerin, about 5.72 g propylene glycol and 4.60 g of 0.2 M sodium phosphate buffer, pH 7.9, with sterilized water q.s. to 16 mL.

In one embodiment, a single dose liquid formulation of the present invention comprises about 330 mg bevirimat dimeglumine, about 2.26 g ethanol, about 2.42 g glycerin, about 5.97 g propylene glycol and 4.80 g of 0.2 M sodium phosphate buffer, pH 7.9, with sterilized water q.s. to 16 mL.

In one embodiment, a single dose liquid formulation of the present invention comprises about 330 mg bevirimat dimeglumine, about 1.90 g ethanol, about 2.02 g glycerin, about 4.99 g propylene glycol and 4.00 g of 0.2 M sodium phosphate buffer, pH 7.9, and 0.80 g xylitol, with sterilized water q.s. to 16 mL.

In one embodiment, a single dose liquid formulation of the present invention comprises about 330 mg bevirimat dimeglumine, about 1.98 g ethanol, about 2.12 g glycerin, about 5.22 g propylene glycol, 4.20 g of 0.2 M sodium phosphate buffer, pH 7.9, and 0.84 g xylitol, with sterilized water q.s. to 16 mL.

In one embodiment, a single dose liquid formulation of the present invention comprises about 330 mg bevirimat dimeglumine, about 2.07 g ethanol, about 2.22 g glycerin, about 5.47 g propylene glycol, 4.40 g of 0.2 M sodium phosphate buffer, pH 7.9, and 0.88 g xylitol, with sterilized water q.s. to 16 mL.

In one embodiment, a single dose liquid formulation of the present invention comprises about 330 mg bevirimat dimeglumine, about 2.17 g ethanol, about 2.32 g glycerin, about 5.72 g propylene glycol, 4.60 g of 0.2 M sodium phosphate buffer, pH 7.9, and 0.92 g xylitol, with sterilized water q.s. to 16 mL.

In one embodiment, a single dose liquid formulation of the present invention comprises about 330 mg bevirimat dimeglumine, about 2.26 g ethanol, about 2.42 g glycerin, about 5.97 g propylene glycol, 4.80 g of 0.2 M sodium phosphate buffer, pH 7.9, and 0.96 g xylitol, with sterilized water q.s. to 16 mL.

In one embodiment, a single dose liquid formulation of the present invention comprises about 330 mg bevirimat dimeglumine, about 1.90 g ethanol, about 2.02 g glycerin, about 4.99 g propylene glycol and 4.00 g of 0.2 M sodium phosphate buffer, pH 7.9, 0.08 g menthol and 0.80 g xylitol, with sterilized water q.s. to 16 mL.

In one embodiment, a single dose liquid formulation of the present invention comprises about 330 mg bevirimat dimeglumine, about 1.98 g ethanol, about 2.12 g glycerin, about 5.22 g propylene glycol, 4.20 g of 0.2 M sodium phosphate buffer, pH 7.9, 0.08 g menthol and 0.84 g xylitol, with sterilized water q.s. to 16 mL.

In one embodiment, a single dose liquid formulation of the present invention comprises about 330 mg bevirimat dimeglumine, about 2.07 g ethanol, about 2.22 g glycerin, about 5.47 g propylene glycol, 4.40 g of 0.2 M sodium phosphate buffer, pH 7.9, 0.09 g menthol and 0.88 g xylitol, with sterilized water q.s. to 16 mL.

In one embodiment, a single dose liquid formulation of the present invention comprises about 330 mg bevirimat dimeglumine, about 2.17 g ethanol, about 2.32 g glycerin, about 5.72 g propylene glycol, 4.60 g of 0.2 M sodium phosphate buffer, pH 7.9, 0.09 g menthol and 0.92 g xylitol, with sterilized water q.s. to 16 mL.

In one embodiment, a single dose liquid formulation of the present invention comprises about 330 mg bevirimat dimeglumine, about 2.26 g ethanol, about 2.42 g glycerin, about 5.97 g propylene glycol, 4.80 g of 0.2 M sodium phosphate buffer, pH 7.9, 0.10 g menthol and 0.96 g xylitol, with sterilized water q.s. to 16 mL.

In one embodiment, a single dose liquid formulation of the present invention comprises about 330 mg bevirimat dimeglumine, about 1.90 g ethanol, about 2.02 g glycerin, about 4.99 g propylene glycol and 4.00 g of 0.2 M sodium phosphate buffer, pH 7.9, 0.03 g mint and 0.80 g xylitol, with sterilized water q.s. to 16 mL.

In one embodiment, a single dose liquid formulation of the present invention comprises about 330 mg bevirimat dimeglumine, about 1.98 g ethanol, about 2.12 g glycerin, about 5.22 g propylene glycol, 4.20 g of 0.2 M sodium phosphate buffer, pH 7.9, 0.03 g mint and 0.84 g xylitol, with sterilized water q.s. to 16 mL.

In one embodiment, a single dose liquid formulation of the present invention comprises about 330 mg bevirimat dimeglumine, about 2.07 g ethanol, about 2.22 g glycerin, about 5.47 g propylene glycol, 4.40 g of 0.2 M sodium phosphate buffer, pH 7.9, 0.04 g mint and 0.88 g xylitol, with sterilized water q.s. to 16 mL.

In one embodiment, a single dose liquid formulation of the present invention comprises about 330 mg bevirimat dimeglumine, about 2.17 g ethanol, about 2.32 g glycerin, about 5.72 g propylene glycol, 4.60 g of 0.2 M sodium phosphate buffer, pH 7.9, 0.04 g mint and 0.92 g xylitol, with sterilized water q.s. to 16 mL.

In one embodiment, a single dose liquid formulation of the present invention comprises about 330 mg bevirimat dimeglumine, about 2.26 g ethanol, about 2.42 g glycerin, about 5.97 g propylene glycol, 4.80 g of 0.2 M sodium phosphate buffer, pH 7.9, 0.04 g mint and 0.96 g xylitol, with sterilized water q.s. to 16 mL.

In one embodiment, a single dose liquid formulation of the present invention comprises from about 325 mg to 475 mg bevirimat dimeglumine and a combination of carrier materials comprising ethanol, propylene glycol and glycerin wherein the weight/volume percent ratio of ethanol:propylene glycol:glycerin is about 1: from about 2 to about 4:about 1 and wherein the aggregate amount of the single dose liquid formulation is about 16-18 g.

In one embodiment, a single dose liquid formulation of the present invention comprises from about 325 mg to about 475 mg bevirimat dimeglumine and a combination of carrier materials comprising ethanol, propylene glycol and glycerin wherein the weight/volume percent ratio of ethanol:propylene glycol:glycerin is about 1: from about 2 to about 4:about 1 and wherein the aggregate amount of the single dose liquid formulation is about 24-26 g.

In one embodiment, a single dose liquid formulation of the present invention comprises from about 325 mg to about 475 mg bevirimat dimeglumine and a combination of carrier materials comprising ethanol, propylene glycol and glycerin wherein the weight/volume percent ratio of ethanol:propylene glycol:glycerin is about 1: from about 2 to about 4:about 1, and the amount of xylitol is from about 0.7 g to about 1 g, wherein the aggregate amount of the single dose liquid formulation is about 16-18 g.

In one embodiment, a single dose liquid formulation of the present invention comprises from about 325 mg to about 475 mg bevirimat dimeglumine and a combination of carrier materials comprising ethanol, propylene glycol and glycerin wherein the weight/volume percent ratio of ethanol:propylene glycol:glycerin is about 1: from about 2 to about 4:about 1, and the amount of xylitol is from about 1.0 g to about 1.5 g, wherein the aggregate amount of the single dose liquid formulation is about 24-26 g.

In one embodiment, a single dose liquid formulation of the present invention comprises from about 325 mg to about 475 mg bevirimat dimeglumine and a combination of carrier materials comprising ethanol, propylene glycol and glycerin wherein the weight/volume percent ratio of ethanol:propylene glycol:glycerin is about 1:from about 2 to about 4:about 1, the amount of menthol is from about 0.05 g to about 0.15 g and the amount of xylitol is from about 0.7 g to about 1 g, wherein the aggregate amount of the single dose liquid formulation is about 16-18 g.

In one embodiment, a single dose liquid formulation of the present invention comprises from about 325 mg to about 475 mg bevirimat dimeglumine and a combination of carrier materials comprising ethanol, propylene glycol and glycerin wherein the weight percent ratio of ethanol:propylene glycol:glycerin is about 1:from about 2 to about 4:about 1, the amount of menthol is from 0.07 g to about 0.25 g and the amount of xylitol is from about 1.0 g to about 1.5 g, wherein the aggregate amount of the single dose liquid formulation is about 24-26 g.

The final volume of 16 mL described for the specific liquid formulations above can be modified as desired, suitable final volumes being within the range of from 15 mL to 40 mL. If the final volume of the liquid formulation is decreased or increased compared to the final volume of 16 mL exemplified above, the amounts of the components other than the bevirimat drug substance should be adjusted accordingly. In other words, the weights of the components other than the bevirimat drug substance are proportional to the total volume of the liquid formulation.

In one embodiment, useful orally deliverable liquid compositions of the present invention include those having from about 0.01 to about 0.42 w/v-% of bevirimat dimeglumine; from about 10 to about 15 w/v-% of ethanol; from about 26 to about 38 w/v-% of propylene glycol; from about 10 to about 17 w/v-% of glycerin; from about 20 to about 31 w/v-% of 0.2 M phosphate buffer (especially 0.2 M sodium phosphate buffer), pH 7.9; from about 4 to about 7 w/v-% of xylitol; from about 0.4 to about 0.7 w/v-% of menthol or from about 0.1 to about 0.8 w/v-% of mint; and sterilized water q.s. to from about 30 to about 54 w/v-%.

In one embodiment, useful orally deliverable liquid compositions of the present invention include those having from about 0.08 to about 0.35 w/v-% of bevirimat dimeglumine; from about 11 to about 14 w/v-% of ethanol; from about 27 to about 37 w/v-% of propylene glycol; from about 11 to about 16 w/v-% of glycerin; from about 22 to about 30 w/v-% of 0.2 M phosphate buffer (especially 0.2 M sodium phosphate buffer), pH 7.9; from about 4.5 to about 6.5 w/v-% of xylitol; from about 0.45 to about 0.65 w/v-% of menthol or from about 0.2 to about 0.6 w/v-% of mint; and sterilized water q.s. to from about 32 to about 48 w/v-%.

In one embodiment, useful orally deliverable liquid compositions of the present invention include those having from about 0.18 to about 0.25 w/v-% of bevirimat dimeglumine; from about 12.4 to about 13.6 w/v-% of ethanol; from about 32.6 to about 36.0 w/v-% of propylene glycol; from about 13.2 to about 14.6 w/v-% of glycerin; from about 26.2 to about 29.0 w/v-% of 0.2 M phosphate buffer (especially 0.2 M sodium phosphate buffer), pH 7.9; from about 5.25 to about 6.0 w/v-% of xylitol; from about 0.53 to about 0.60 w/v-% of menthol or from about 0.25 to about 0.50 w/v-% of mint; and sterilized water q.s. to from about 33.0 to about 40.0 w/v-%.

In one aspect of the invention, the orally deliverable liquid formulation of the present invention preferably has any of the compositions A-E and D-L Menthol shown below in Table 24 or any of the compositions AA-EE, DD-L Menthol, and DD Mint shown below in Table 27.

In one aspect of the present invention, a unit dose formulation of the present invention having 200 mg of bevirimat free acid equivalents, comprises about 330 mg bevirimat dimeglumine, about 1.90 g ethanol, about 2.02 g glycerin, about 4.99 g propylene glycol, 4.00 g of 0.2 M sodium phosphate buffer, pH 7.9, 0.08 g menthol, 0.80 g xylitol, and sterilized water q.s. to about 2.66 g.

In one aspect of the present invention, a unit dose formulation of the present invention having 200 mg of bevirimat free acid equivalents, comprises about 330 mg bevirimat dimeglumine, about 1.98 g ethanol, about 2.12 g glycerin, about 5.22 g propylene glycol, 4.20 g of 0.2 M sodium phosphate buffer, pH 7.9, 0.08 g menthol, 0.84 g xylitol, and sterilized water q.s. to about 2.15 g.

In one aspect of the present invention, a unit dose formulation having 200 mg of bevirimat free equivalents, comprises about 330 mg bevirimat dimeglumine, about 2.07 g ethanol, about 2.22 g glycerin, about 5.47 g propylene glycol, 4.40 g of 0.2 M sodium phosphate buffer, pH 7.9, 0.09 g menthol, 0.88 g xylitol, and sterilized water q.s. to about 1.51 g.

In one aspect of the present invention, a unit dose formulation having 200 mg of bevirimat free equivalents, comprises about 330 mg bevirimat dimeglumine, about 2.17 g ethanol, about 2.32 g glycerin, about 5.72 g propylene glycol, 4.60 g of 0.2 M sodium phosphate buffer, pH 7.9, 0.09 g menthol, 0.92 g xylitol, and sterilized water q.s. to about 0.83 g.

In one aspect of the present invention, a unit dose formulation having 200 mg of bevirimat free equivalents, comprises about 330 mg bevirimat dimeglumine, about 2.26 g ethanol, about 2.42 g glycerin, about 5.97 g propylene glycol, 4.80 g of 0.2 M sodium phosphate buffer, pH 7.9, 0.10 g menthol, and 0.96 g xylitol.

### Dispersion Profile

In one embodiment of the present invention, about 50% of the bevirimat present in the liquid formulation disperses in simulated gastric fluid within about 15 minutes.

In one embodiment of the present invention, about 70% of the bevirimat present in the liquid formulation disperses in simulated gastric fluid within about 15 minutes.

In one embodiment of the present invention, about 80% of the bevirimat present in the liquid formulation disperses in simulated gastric fluid within about 30 minutes.

In one embodiment of the present invention, about 90% of the bevirimat present in the liquid formulation disperses in simulated gastric fluid within about 45 minutes.

In one embodiment of the present invention, substantially all of the bevirimat present in the liquid formulation disperses in simulated gastric fluid within about 60 minutes.

In one embodiment of the present invention, the bevirimat liquid formulation exhibits long term stability over 2 years under ambient conditions.

In one embodiment of the present invention, the bevirimat liquid formulation exhibits long term stability over 2 years at 40° C. at 75% RH.

In one embodiment of the present invention, the bevirimat liquid formulation exhibits long term stability over 6 months at 40° C. at 75% RH.

In one embodiment of the present invention, the bevirimat liquid formulation exhibits long term stability over 4 months at 40° C. at 75% RH.

In one embodiment of the present invention, the bevirimat liquid formulation exhibits long term stability over 2 months at 40° C. at 75% RH.

In some embodiments, the present invention comprises a formulation where a detectable amount of the bevirimat drug substance is dispersed, dissolved or solubilized in the solvent liquid.

In some embodiments, the present invention comprises a formulation where greater than 75% of the bevirimat drug substance is dispersed, dissolved or solubilized in the solvent liquid.

In some embodiments, the present invention comprises a formulation where at least about 85% of the bevirimat drug substance is present in the pharmaceutically acceptable solvent in dissolved or solubilized form.

In some embodiments, the present invention comprises a formulation where greater than 95% of the bevirimat drug substance is dispersed, dissolved or solubilized in the solvent liquid.

In some embodiments, the present invention comprises a formulation where substantially all of the bevirimat drug substance is dispersed, dissolved or solubilized in the solvent liquid.

### Forms

Liquid dosage forms for oral administration can include pharmaceutically acceptable emulsions, solutions, suspensions, gels, syrups, and elixirs containing inert diluents commonly used in the art, such as water. Such compositions can also comprise, for example, active pharmaceutical ingredient, solvents, co-solvents, surfactants, colorants, anti-oxidants, emulsifying agents, suspending agents, sweetening agents, flavoring agents, perfuming agents, and preservatives.

In some embodiments, the liquid formulation of the present invention is a suspension of fine solid bevirimat drug substance particles in a liquid. The liquid can be a lipophilic or hydrophilic liquid or a mixture of several liquids. Any liquid can further comprise dissolved ingredients. By decreasing the particle size of the bevirimat drug substance, the surface area available for digestion and drug release is enhanced.

In some embodiments, the liquid formulation of the present invention is an emulsion. Because the aqueous solubility of the bevirimat drug substance is very low, the emulsion is preferably a dispersion of a lipophilic phase (e.g., a solution of the bevirimat drug substance in a lipophilic liquid) in an aqueous phase (oil-in-water emulsion). The emulsion may comprise additional components such as stabilizers, surfactants, emulsifiers, and thickeners. In some embodiments, the emulsion is a microemulsion. In some embodiments, the emulsion is a nanoemulsion. Preferred microemulsions and nanoemulsions are isotropic, transparent and thermodynamically stable. The average size of the dispersed droplets is in a microemulsion typically range from about 100 μM to about 1 μM. The average size of the dispersed droplets is in a nanoemulsion typically range from about 100 nM to about 1 nM.

Although the liquid formulations such as solutions, emulsions and suspensions can be packed in larger bottles for multiple doses, in some embodiments the liquid formulation may be enclosed in a unit dosage form, such as a capsule. Illustrative encapsulated formulations include softgel capsules. Softgel capsules comprise a liquid or semisolid matrix inside an outer shell, for example, a gelatin shell. The bevirimat drug substance itself may be either in solution, suspension or emulsion in the matrix. The characteristics of the matrix may be hydrophilic (for example polyethylene glycols) or lipophilic (such as triglyceride vegetable oils), or a mixture of both hydrophilic and lipophilic ingredients. In some embodiments, the liquid formulations are enclosed in an article suitable for dispensing a bevirimat drug substance, such as a hollow spoon comprising a premeasured amount of a bevirimat drug substance suitable for self administration by a patient.

### Preparation of Bevirimat

The bevirimat drug substance of the pharmaceutical compositions of the present invention can be prepared using the methods set forth in U.S. Pat. No. 5,679,828; US Patent Application Publication No. US 2005/0239748 A1 (U.S. patent Ser. No. 11/081,802); US Patent Application Publication No. US 2006/0252704 A1 (U.S. patent Ser. No. 11/401,960); and US Patent Application Publication No. US 2007/0203103 A1 (U.S. patent Ser. No. 11/640,488), all of such disclosures are incorporated by reference in their entirety.

### Methods of Treatment

The present invention further encompasses the use of bevirimat liquid dosage formulations in the manufacture of a medicament for the treatment of HIV-1 infection.

The present invention also is directed to therapeutic methods of treating a condition or disorder where treatment with a maturation inhibitor is indicated, the methods comprising the oral administration of one or more of the pharmaceutical compositions previously described above to a patient in need thereof. The dosage regimen to treat HIV-1 corresponds to once-a-day dosage forms. Factors useful in determining the dose required by a patient in need of a bevirimat drug substance include the type, age, weight, sex, diet, and medical condition of the patient and the severity of the disease. As the dosage forms are liquid dosage forms, the dosage regimen actually employed can vary widely and therefore deviate from the preferred dosage regimen set forth herein.

In some cases, initial treatment of a patient suffering from a condition or disorder where treatment with an HIV-1 maturation inhibitor is indicated can begin with the dosages indicated above. Treatment is generally continued as necessary over a period of several weeks to several months or years until the condition or disorder has been controlled or eliminated. Patients undergoing treatment with the compositions disclosed herein can be routinely monitored by any of the methods well known in the art to determine the effectiveness of therapy by known methods, including viral load analysis or CD4 counts. Continuous analysis of such data permits modification of the treatment regimen during therapy so that optimal effective amounts of compounds of the present invention are administered at any point in time, and so that the duration of treatment can be determined as well. In this way, the treatment regimen/dosing schedule can be rationally modified over the course of therapy so that the lowest amount of bevirimat exhibiting satisfactory effectiveness is administered, and so that administration is continued only so long as is necessary to successfully treat the condition or disorder.

The present invention relates to compositions, wherein upon administration of a single 200 mg dose (free acid equivalent) of the bevirimat drug substance to a human patient: (i) an AUC_{0-∞} (μg·hr/mL bevirimat) of from about 800 to about 2700; and, (ii) a Tₘₐₓ of less than about 6 hours after oral administration of the bevirimat drug substance are achieved. Preferably, the AUC_{0-∞} (μg·hr/mL bevirimat) is from about 1000 to about 2100, and more preferably about 2000.

The present invention also relates to compositions, wherein upon administration of a single 200 mg dose (free acid equivalent) of the bevirimat drug substance to a human patient: (i) an AUC_{0-∞} (μg·hr/mL bevirimat) of from about 1000 to about 2500; and, (ii) a Tₘₐₓ of less than about 4 hours after oral administration of the bevirimat drug substance are achieved. Preferably, the AUC_{0-∞} (μg·hr/mL bevirimat) is from about 1,500 to about 2,000.

Further, the present invention relates to a method for achieving an AUC_{0-∞} (μg·hr/mL bevirimat free acid) of from about 1,000 to about 10,000 and a Tₘₐₓ of less than about 4 hours after oral administration of a single oral liquid dosage form, comprising: administering the dosage form comprising: (i) about 200 mg to about 1,000 mg free acid equivalents of a bevirimat drug substance; (ii) a solvent; and (iii) a polymer, to a patient in need of said dosage form. Preferably, the solvent comprises propylene glycol, ethanol, and glycerin. Preferably, the presence of the polymer is optional.

Further, the present invention relates to a method for achieving a Cₘₐₓ (μg bevirimat free acid equivalents/mL) of from about 100 to about 300 with a Tₘₐₓ of less than about 4 hours, comprising: (a) administration of a single oral liquid dosage form comprising: (i) about 200 mg to about 750 mg free acid equivalents of a bevirimat drug substance; (ii) a solvent; and (iii) a polymer. Preferably, the Cₘₐₓ is about 200.

The present invention also relates to a composition, wherein upon administration of a single dose of the bevirimat drug substance to a human patient a Cₘᵢₙ (μg bevirimat/mL) of about 70 to about 130 is achieved. Preferably, the Cₘᵢₙ is about 100.

### DEFINITIONS

The terms “a” and “an” refer to one or more.

The term “FAE” means “free acid equivalents”.

The term “BVM” means “bevirimat”.

As used herein, the phrase “aggregate amount” is the total weight of all components in a single dose oral liquid formulation of the present invention.

As used herein, the term “buffer” refers to a buffering agent, either in a solid form or in an aqueous solution.

The term “amorphous” as applied to bevirimat dimeglumine herein refers to a solid state form wherein the bevirimat dimeglumine molecules (i) are present in a disordered arrangement, (ii) do not form a distinguishable crystal lattice or unit cell, or (iii) do not exhibit long range order. When subjected to X-ray powder diffraction, amorphous bevirimat dimeglumine does not produce a diffraction pattern characteristic of a crystalline form.

The term “crystalline form” as applied to bevirimat dimeglumine herein refers to a solid state form wherein the bevirimat dimeglumine molecules are arranged to form a distinguishable crystal lattice (i) comprising distinguishable unit cells, and (ii) yielding diffraction peaks when subjected to X-ray radiation.

The term “bevirimat drug substance” refers to 3-O-(3′,3-dimethylsuccinyl)betulinic acid (bevirimat), mono-salts of bevirimat, di-salts of bevirimat, and combinations thereof, and can refer to: (i) the free acid of bevirimat; (ii) unformulated mono- or di-salt of bevirimat, such as bevirimat meglumine, bevirimat dimeglumine, bevirimat monopotassium, bevirimat dipotassium, bevirimat monosodium, and bevirimat disodium, present in a step in the manufacture of a pharmaceutical composition, or (ii) a mono- or a di-salt of bevirimat, such as bevirimat meglumine, bevirimat dimeglumine, bevirimat monopotassium, bevirimat dipotassium, bevirimat monosodium, and bevirimat disodium, present as an ingredient of a pharmaceutical composition, or combinations thereof.

The term “phase pure” refers to purity with respect to other solid state forms of bevirimat dimeglumine and does not necessarily imply a high degree of chemical purity with respect to other compounds.

The term “weight percent” as used herein means the weight percent of a specified ingredient based upon the total weight of all ingredients of the composition.

The terms “w/v-%” and “weight/volume percent” as used herein mean the weight of a component (in grams) for every 100 mL of the liquid pharmaceutical composition of the present invention.

The term “dose unit” means the amount of bevirimat drug substance administered in a 24 hour period; thus a “dose unit” can consist of a single dosage form, or a “dose unit” can consist of multiple discrete dosage forms administered simultaneously or with a time frame allowing for. The exact dose is dependent on the route of administration, age, gender and body weight of the patient. The administration of the dose unit can be carried out by: (i) single administration in the form of an individual dosage form; (ii) simultaneous administration of multiple dosage forms; or (iii) consecutive administration of multiple dosage forms at specific intervals so long as the intervals allow for an overlap of therapeutic effect.

The term “solvent liquid” herein means a component or a plurality of components of the liquid medium in which a particular drug is dispersed. Thus the “solvent liquid” includes not only one or more solvents but also optionally additional excipients such as co-solvents, surfactants, co-surfactants, antioxidants, sweeteners, complexation agents, flavoring agents, and colorants.

The term “solubilizer” means a component or a plurality of components that increase the solubility of bevirimat or bevirimat dimeglumine in a solvent relative to the solubility of bevirimat or bevirimat dimeglumine in the same solvent under the storage conditions. Exemplary solubilizers include surfactants, co-surfactants, co-solvents, and, complexation agents. Other exemplary solubilizers include water soluble organic solvents, non-ionic surfactants, ionic surfactants, water insoluble lipids, organic liquids, cyclodextrins, phospholipids, and combinations thereof. Exemplary water soluble organic solvents include PEG 300, PEG 400, ethanol, propylene glycol, glycerin, N-methyl-2-pyrrolidone, dimethylacetamine, dimethylsulfoxide, and combinations thereof. Exemplary non-ionic surfactants include Cremophor EL, Cremophor RH 40, Cremophor RH 60, Vitamin E-TPGS, d-α-tocopherol, PEG 1000 succinate, polysorbate 20, polysorbate 80, Solutol HS15, sorbitan monooleate, poloxamer 407, Labrafil M-1944CS, Labrafil M-2125CS, Labrasol, Gellucire 44/14. Softigen 767, mono- and di-fatty acid esters of PEG 300, 400, or 1750, and combinations thereof. Exemplary water insoluble lipids include castor oil, corn oil, cottonseed oil, olive oil, peanut oil, peppermint oil, safflower oil, sesame oil, soybean oil, hydrogenated vegetable oil, hydrogenated soybean oil, medium chain triglycerides of coconut oil or palm seed oil, and combinations thereof. Medium chain triglycerides (“MCT”) are medium-chain (about 6 to about 12 carbon) fatty acid esters of glycerol. Illustrative MCTs include caproic (C6), caprylic (C8), capric (C10) and lauric acid (C12). Exemplary organic liquids include beeswax, d-α-tocopherol, oleic acid, medium chain mono- and di-glycerides, and combinations thereof. Exemplary cyclodextrins include α-cyclodextrin, β-cyclodextrin, hyrdoxypropyl-β-cyclodextrin, sulfobutylether-β-cyclodextrin, and combinations thereof. Exemplary phospholipids include hydrogenated soy phosphatidylcholine, distearoylphosphatidylglycerol, L-α-dimyristoylphosphatidylcholine, L-α-dimyristoylphosphatidylglycerol, and combinations thereof.

It is understood that a particular excipient or carrier material can impart more than one property upon a particular liquid formulation. For example, ethanol could be present in a given formulation as a part of the solvent liquid at concentrations sufficient to impart solubilizing properties to the liquid formulation; in this example, the ethanol would be properly defined as both a solvent liquid and as a solubilizer.

The term “simulated gastric fluid” or “SGF” means an aqueous solution prepared according to the USP. Generally, SGF is prepared by dissolving 2.0 g NaCl (58.44 g/mol), 7.0 mL conc. HCL (11.6N) in sufficient water to make 1 L. Final composition is 0.08 N HCl, 34 mM NaCl, pH 1.2. When used in media comprising pepsin, 3.2 g of pepsin (sp act 800-2500 U/mg protein is added to SGF prepared as described above.

By “polymeric” is meant that the material is made up of a series of similar repeat units ranging in number from 5 to 10 up to many thousands. The repeat units essentially all may be the same, as would be the case for a polymer such as polyvinylpyrrolidone, or they may vary as would be the case for a substituted cellulosic polymer.

The term “EC₅₀” means the drug concentration that results in a 50% inhibition of virus replication.

The term “EC₉₀” means the drug concentration that results in a 90% inhibition of virus replication.

The term “D₉₀” means the diameter where 10 mass % of the particles have a larger equivalent diameter, and the other 90 mass % have a smaller equivalent diameter

The term “D₅₀” means the diameter where 50 mass % of the particles have a larger equivalent diameter, and the other 50 mass % have a smaller equivalent diameter. The term “Cₘₐₓ” means the maximum observed concentration.

The term “Tₘₐₓ” means the time at which Cₘₐₓ occurred.

The term “T_{1/2}” means half-life.

The term “Cₘₐₓ/AUC_{(0-LQC)}” means the rate of absorption.

The term “AUCₐₗₗ” means area Under the Curve which is calculated using all data points.

The term “Dnzed” means dose normalized AUC which is calculated by dividing the AUC by the dose.

The term “BLOQ” means below the limit of quantitation (50 ng/mL).

The term “ND” means not determined

The term “PG” means propylene glycol.

The term “PEG” means polyethylene glycol.

The term “IV” means intravenous.

The term “carrier material” means material included in a pharmaceutical composition to impart certain desirable properties.

The amount of drug incorporated in a dosage form of the invention can be selected according to known principles of pharmacy. A therapeutically effective amount of drug is specifically contemplated. The term “therapeutically effective amount” as used herein refers to an amount of bevirimat that is sufficient to elicit the required or desired therapeutic response. Typically, the bevirimat will be present in a total amount of about 1% to about 75% by weight of the composition.

As used herein in connection with a measured quantity, “about” refers to the normal variations in that measured quantity, as expected by the skilled artisan making the measurement and exercising a level of acre commensurate with the objective of measurement and the precision of the measuring equipment. When used in relation with amount of time, “about” can have its ordinary meaning.

### EXAMPLES

The following examples are illustrative, but not limiting, of the compositions and methods of the present invention. Suitable modifications and adaptations of the variety of conditions and parameters normally encountered and obvious to those skilled in the art are within the spirit and scope of the invention.

Unless otherwise specified, concentrations of bevirimat described in the Examples refer to concentration of the bevirimat free di-acid.

### Example 1

### Preparation of the di-(N-methyl-D-glucamine) Salt of Bevirimat (“Bevirimat Dimeglumine”)

N-methyl-D-glucamine is dissolved in 250 mL methyl alcohol. About 0.5 equivalents of DSB is added and allowed to sit overnight until the suspension becomes clear. The solvent is removed with a nitrogen gas stream. A thick, colorless oil will form. 200 mL methyl alcohol is added to dissolve the oil. Slow addition of 200 mL diethyl ether to the swirling mixture affords a white solid. The solid material can be isolated by vacuum filtration.

### Example 2

### Bevirimat Dimeglumine Form I

In one aspect the present invention employs bevirimat dimeglumine Form I which can be prepared by following the following method: saturated solutions are first prepared by agitating bevirimat dimeglumine in contact with a first suitable solvent at the saturation temperature. The mother liquor is separated from any residual solids by filtration. The mother liquor is then diluted with a second suitable solvent, when necessary, and heated above the saturation temperature (overheated and unsaturated) of the resulting solvent system to dissolve any remaining solids. The temperature of the solution is then adjusted to the growth temperature, i.e., a temperature capable of allowing solidification of bevirimat dimeglumine in the resulting solvent system.

In one embodiment, bevirimat dimeglumine Form I is crystallized from a suitable solvent, such as, but not limited to, DMF, as exemplified below. In other embodiments, bevirimat dimeglumine Form I is crystallized by techniques of solvent evaporation or solution saturation well known to those of ordinary skill in the art including but not limited to: introducing a shear flow; introducing a heated element such as heat transfer plates, infrared lamps, microwave systems; distillation with an optional sheer flow wherein the distillation can be performed at atmospheric pressure or under vacuum; static evaporation; reducing the temperature of the bevirimat dimeglumine solution; and, thin film evaporation techniques such as rotary evaporation, spin-off evaporation, rising and falling film evaporation, submerged evaporation, and wiped film evaporation.

### Example 3

### Bevirimat Dimeglumine Form II

Following the general procedure described above, another crystal form of bevirimat dimeglumine can be synthesized using methyl ethyl ketone (MEK) as the solvent system. This crystal form is designated bevirimat dimeglumine Form II and, in some embodiments, is an MEK solvate.

### Example 4

### Amorphous Bevirimat Dimeglumine

Amorphous bevirimat dimeglumine is prepared by dissolving bevirimat dimeglumine in a solvent to form a solution of bevirimat dimeglumine and either (a) adding an anti-solvent, i.e., a solvent in which bevirimat dimeglumine is poorly soluble, or (b) spray drying the solution. Preferably, the solvent is an alcohol, such as methanol, ethanol or isopropanol, or a ketone, such as acetone.

After dissolution of bevirimat dimeglumine in the organic solvent, the organic solvent is removed under reduced or ambient pressure. The evaporation is preferably controlled, and one skilled in the art will appreciate that the conditions of evaporation can affect the quality of the product. The final product can optionally be triturated with an organic solvent such as a saturated hydrocarbon, including inter alia cyclohexane, hexane and heptane, or ethers, including inter alia MTBE (methyl tributyl ether).

### Example 5

The solubility of 12.5 mg/mL bevirimat dimeglumine solution in both simulated gastric fluid, with and without pepsin, and simulated intestinal fluid, with and without pancreatin, was determined over a 4 hour time period at room temperature. All media were prepared according to the USP. Results are shown below in Table 1.

TABLE 1
Vehicle composition and solubility of 12.5 mg/mL bevirimat over 4 hours at RT

| | | Visual appearance/ | Visual appearance/ | Visual appearance/ |
|---|---|---|---|---|
| Formulation | | mg/mL | mg/mL | mg/mL |
| #ID | Vehicle Description | t₀ hours | t₁ hours | t₄ hours |
| 1 | (12.5 mg/mL Bevirimat) | cloudy/0.79 | cloudy/0.81 | cloudy/0.85 |
| | SGF with pepsin | | | |
| 2 | (12.5 mg/mL Bevirimat) | cloudy/0.81 | cloudy/0.81 | cloudy/0.75 |
| | SGF without pepsin | | | |
| 3 | (12.5 mg/mL Bevirimat) | cloudy/0.78 | cloudy/0.79 | cloudy/0.78 |
| | SIF with pancreatin | | | |
| 4 | (12.5 mg/mL Bevirimat) | cloudy/0.92 | cloudy/0.92 | cloudy/1.18 |
| | SIF without pancreatin | | | |

### Example 6

### Preparation of 10% (w/v %) Stock Solution of Vitamin E (TPGS)

10% (w/v %) stock solution of Vitamin E TPGS is prepared as follows:

1. Weigh 10 g TPGS into 100 mL volumetric flask.

2. Add 50 mL WFI and sonicate the solution for approximately 20 minutes or until complete dissolution is observed.

3. QS to 100 mL with WFI.

### Example 7

### Preparation of 1% (w/v %) Stock Solution of Povidone K15

1% (w/v %) stock solution of Povidone K15 is prepared as follows:

1. Weigh 1 g Povidone K15 into 100 mL volumetric flask.

2. Add 50 mL WFI and sonicate the solution for approximately 30 minutes or until complete dissolution is observed.

3. QS to 100 mL with WFI.

### Example 8

### Preparation of 1% (w/v %) Stock Solution of Povidone K30

1% (w/v %) stock solution of Povidone K30 is prepared as follows:

1. Weigh 1 g Povidone K30 into 100 mL volumetric flask.

2. Add 50 mL WFI and sonicate the solution for approximately 30 minutes or until complete dissolution is observed.

3. QS to 100 mL with WFI.

### Example 9

Formulation #2 was dosed at a constant strength of 12.5 mg/mL. Formulation #2 consists essentially of 12.5 mg/mL bevirimat in 15% (v/v) alcohol (ethanol), 10% (v/v) glycerin, 30% (v/v) propylene glycol, 1% (w/v), vitamin E TPGS and has the unit dose composition as specified in Table 2.

TABLE 2
Unit Composition of Liquid Formulation #2

| | Quantity | | |
|---|---|---|---|
| | | Quantity per 16 mL | |
| Component | % (w/w) | dose | |
| Bevirimat Dimeglumine | 2.35% | 350 | mg* (0.35 g) |
| Alcohol (Ethanol) | 12.73% | 2.4 | mL (1.89 g) |
| Glycerin | 12.60% | 1.6 | mL (1.87 g) |
| Propylene Glycol | 33.49% | 4.8 | mL (4.97 g) |
| Vitamin E Polyethylene Glycol | 1.08% | 160 | mg (0.16 g) |
| Succinate (Vitamin E TPGS) | | | |
| Sterile water for irrigation (SWI) | | q.s. to 16 | mL |
| Total | 100.0% | | |

*Equivalent to 200 mg bevirimat free acid

To prepare Formulation #2, the required amount of SWI, bevirimat dimeglumine, alcohol, propylene glycol, glycerin and Vitamin E TPGS solution was measured into an appropriately sized container and mixed until all bevirimat was dissolved.

### Example 10

Formulation #3 was dosed at a constant strength of 12.5 mg/mL. Formulation #3 consists essentially of 12.5 mg/mL bevirimat in 40% (v/v) propylene glycol, 20% (v/v) glycerin, 1% (w/v) Vitamin E TPGS, 0.25% (v/v) Polysorbate 80, 0.2% (w/v) Povidone K15 and has the unit dose composition as specified in Table 3.

TABLE 3
Unit Composition of Liquid Formulation #3

| | Quantity | | |
|---|---|---|---|
| | | Quantity per 16 mL | |
| Component | % w/w | dose | |
| Bevirimat Dimeglumine | 2.23% | 350 | mg* (0.35 g) |
| Glycerin | 23.87% | 3.2 | mL (3.74 g) |
| Propylene Glycol | 42.31% | 6.4 | mL (6.63 g) |
| Vitamin E Polyethylene Glycol | 1.00% | 160 | mg (0.16 g) |
| Succinate (Vitamin E TPGS) | | | |
| Povidone K15 | 0.20% | 32 | mg (0.032 g) |
| Sterile water for irrigation | | q.s. to 16 | mL |
| Total | 100.00% | | |

*Equivalent to 200 mg bevirimat free acid

To prepare Formulation #3, the required amount of SWI, polysorbate 80 propylene glycol, glycerin, Vitamin E TPGS solution, and povidone K15 was measured solution into an appropriately sized container and mixed until all bevirimat was dissolved.

### Example 11

### Preparation of Exemplary Liquid Formulation (25 mg/mL Bevirimat Formulated in 30% (v/v %) Ethanol, 1% (w/v %) Vitamin E-TPGS, 30% (v/v %) Propylene Glycol and 10% (v/v %) Glycerin)

Exemplary liquid formulation containing 25 mg/mL bevirimat formulated in 30% (v/v %) ethanol, 1% (w/v %) Vitamin E-TPGS, 30% (v/v %) propylene glycol and 10% (v/v %)glycerin is prepared as follows:

1. Add 600 μL ethanol to a 25 mL beaker.

2. Add 200 μL Vitamin E-TPGS 10% stock solution to beaker. Swirl gently until homogenous.

3. Add 600 μL propylene glycol and mix the contents by hand until homogenous mixture.

4. Add 200 μL glycerin (by weight: 0.234 g) mix the contents by hand until homogenous mixture.

5. Add 400 μL WFI.

6. Weigh 77.93 mg bevirimat-2NMG salt and transfer to the 25 mL beaker.

7. Place a stir bar in the beaker and stir the solution at RT until bevirimat-2NMG achieved complete dissolution (5 minutes).

### Example 12

### Preparation of Exemplary Liquid Formulation (12.5 mg/mL Bevirimat Formulated in 30% (v/v %) Ethanol, 1% (w/v %) Vitamin E-TPGS, 30% (v/v %) Propylene Glycol and 10% (v/v %) Glycerin)

Exemplary liquid formulation containing 12.5 mg/mL bevirimat formulated in 30% (v/v %) ethanol, 1% (w/v %) Vitamin E-TPGS, 30% (v/v %) propylene glycol and 10% (v/v %) glycerin is prepared as follows:

1. Add 600 μL ethanol to a 25 mL beaker.

2. Add 200 μL Vitamin E-TPGS 10% (w/v %) stock solution to beaker. Swirl gently until homogenous.

3. Add 600 μL propylene glycol, mix the contents by hand until homogenous mixture.

4. Add 200 μL glycerin (by weight: 0.234 g) mix the contents by hand until homogenous mixture.

5. Add 400 μL WFI.

6. Weigh 38.96 mg bevirimat-2NMG salt and transfer to the 25 mL beaker.

7. Place a stir bar in the beaker and stir the solution at RT until bevirimat-2NMG achieved complete dissolution (5 minutes).

### Example 13

### Preparation of Exemplary Liquid Formulation (25 mg Bevirimat Free Acid Equivalents/mL Formulated in 20% (v/v %) Ethanol, 1% (w/v %) Vitamin E-TPGS, 20% (v/v %) Propylene Glycol and 10% (v/v %) glycerin)

Exemplary liquid formulation containing 25 mg bevirimat free acid equivalents/mL formulated in 20% (v/v %) ethanol, 1% (w/v %) Vitamin E-TPGS, 20% (v/v %) propylene glycol and 10% (v/v %)glycerin is prepared as follows:

1. Add 400 μL ethanol to a 25 mL beaker.

2. Add 200 μL Vitamin E-TPGS 10% (w/v %) stock solution to beaker. Swirl gently until homogenous.

3. Add 400 μL propylene glycol, mix the contents by hand until homogenous mixture.

4. Add 200 μL glycerin (by weight: 0.234 g) mix the contents by hand until homogenous mixture.

5. Add 800 μL WFI.

6. Weigh 77.93 mg bevirimat-2NMG salt and transfer to the 25 mL beaker.

7. Place a stir bar in the beaker and stir the solution at RT until bevirimat-2NMG achieved complete dissolution (5 minutes).

### Example 14

### Preparation of Exemplary Liquid Formulation (12.5 Mg Bevirimat Free Acid Equivalents/mL Formulated in 20% (v/v %)Ethanol, 1% (w/v %)Vitamin E-TPGS, 20% (v/v %) Propylene Glycol and 10% (v/v %)Glycerin)

Exemplary liquid formulation containing 12.5 mg bevirimat free acid equivalents/mL formulated in 20% (v/v %) ethanol, 1% (w/v %)Vitamin E-TPGS, 20% (v/v %) propylene glycol and 10% (v/v %) glycerin is prepared as follows:

1. Add 400 μL ethanol to a 25 mL beaker.

2. Add 200 μL Vitamin E-TPGS 10% (w/v %)stock solution to beaker. Swirl gently until homogenous.

3. Add 400 μL propylene glycol, mix the contents by hand until homogenous mixture.

4. Add 200 μL glycerin (by weight: 0.234 g) mix the contents by hand until homogenous mixture.

5. Add 800 μL WFI.

6. Weigh 38.96 mg bevirimat-2NMG salt and transfer to the 25 mL beaker.

7. Place a stir bar in the beaker and stir the solution at RT until bevirimat-2NMG achieved complete dissolution (5 minutes).

### Example 15

### Preparation of Exemplary Liquid Formulation (25 mg Bevirimat Free Acid Equivalents/mL Formulated in 20% (v/v %) Ethanol, 1% (w/v %)Vitamin E-TPGS, 20% (v/v %) Propylene Glycol and 10% (v/v %)Glycerin)

Exemplary liquid formulation containing 25 mg bevirimat free acid equivalents/mL formulated in 20% (v/v %) ethanol, 1% (w/v %) Vitamin E-TPGS, 20% (v/v %) propylene glycol and 10% (v/v %) glycerin is prepared as follows:

1. Add 800 μL ethanol to a 25 mL beaker.

2. Add 200 μL Vitamin E-TPGS 10% (w/v %) stock solution to beaker. Swirl gently until homogenous.

3. Add 400 μL propylene glycol, mix the contents by hand until homogenous mixture.

4. Add 200 μL glycerin (by weight: 0.234 g) mix the contents by hand until homogenous mixture.

5. Add 400 μL WFI.

6. Weigh 77.92 mg bevirimat-2NMG salt and transfer to the 25 mL beaker.

7. Place a stir bar in the beaker and stir the solution at RT until bevirimat-2NMG achieved complete dissolution (5 minutes).

### Example 16

### Preparation of 12.5 mg Bevirimat Free Acid Equivalents/mL Formulated in 40% (v/v %) PG, 20% (v/v %) Glycerin, 1% (w/v %) Vitamin E-TPGS, 0.25% (v/v %) Tween80 and 0.2% (w/v %)Povidone K15

The title composition is prepared as follows:

1. Add 800 μL PG to a 10 mL Lyo vial.

2. Add 400 μL glycerin (by weight: 0.468 g) mix the contents by hand until homogenous mixture.

3. Add 200 μL Vitamin E-TPGS 10% (w/v %) stock solution to the Lyo vial. Swirl gently until homogenous.

4. Add 5 μL Tween80 (5.350 mg by weight) to the Lyo vial and swirl gently until homogenous.

5. Add 400 μL it Povidone K15 1% (w/v %) stock (see Appendix for preparation of 1% stock Povidone K15) and mix the contents by hand until homogenous mixture.

6. Add 195 μL WFI and mix the contents by hand until homogenous mixture.

7. Weigh 38.96 mg bevirimat-2NMG salt and transfer to the Lyo vial.

8. Place a stir bar in the beaker and stir the solution at RT until bevirimat-2NMG achieved complete dissolution (˜10 minutes).

### Example 17

### Preparation of Iv Formulation

The title formulation is prepared as follows:

1. Weigh out the required amount of bevirimat dimeglumine into an appropriately sized container.

2. Measure out and add the required amount of a 10% HPBCD solution to the container.

3. Stir with a stir-bar @ 300-500 rpm until the compound is completely dissolved (approximately one hour).

4. Weigh out the required amount of mannitol powder and add this to the drug solution.

5. Stir with a stir-bar @ 300-500 rpm until the mannitol is completely dissolved (approximately 15 min)

6. Sterile filter the solution through a 0.45 mm membrane filter.

### Example 18

To determine the stability of formulations, a number of formulations were prepared and dissolved in a series of ternary and quaternary solvents. The formulations were visually scored for cloudiness and formation of gels. In some embodiments, preferred formulations remain clear after 2 hours. In some embodiments, preferred formulations remain clear after 24 hours. In some embodiments, preferred formulations remain clear after 48 hours. In some embodiments, preferred formulations did not form gels within 2 hours. In some embodiments, preferred formulations did not form gels within 24 hours. In some embodiments, preferred formulations did not form gels within 48 hours. Results are shown below in Table 4.

TABLE 4
Equilibrium solubility of 50 mg/mL bevirimat in organic mixtures upon 48
hours at RT. All liquids are expressed as (v/v %). All solids are expressed
as (w/v %) including Poloxamers and Povidones

| Vehicle | | Visual observation (Bevirimat free acid mg/mL) | | |
|---|---|---|---|---|
| ID | Vehicle Composition | 2 hr | 24 hr | 48 hr |
| 1 | 10% Ethanol + 20% | Gel-like | N/A | N/A |
| | Propylene glycol + | solution | | |
| | 10% Polysorbate | | | |
| 2 | 10% Ethanol + 20% | Gel-like | N/A | N/A |
| | Propylene glycol + | solution | | |
| | 10% Acconon | | | |
| 3 | 10% Ethanol + 20% | Gel-like | N/A | N/A |
| | Propylene glycol + | solution | | |
| | 10% Polysorbate + | | | |
| | 10% Acconon | | | |
| 4 | 20% Propylene glycol + | N/A | N/A | N/A |
| | 20% | | | |
| | Polysorbate + 20% | | | |
| | Acconon | | | |
| 5 | 25% Povidone K30 + | Clear solution, | Clear solution, | Clear solution, |
| | 10% PG + 50% Ethanol | 57.9 mg/mL | 57.4 mg/mL | 52.3 mg/mL |
| 6 | 25% Acconon MC-8 + | Clear solution, | Clear solution, | Clear solution, |
| | 10% PG + 50% Ethanol | 50.8 mg/mL | 50.5 mg/mL | 50.0 mg/mL |
| 7 | 25% Tween80 + 10% | Clear solution, | Clear solution, | Clear solution, |
| | Propylene glycol + | 54.3 mg/mL | 53.6 mg/mL | 51.7 mg/mL |
| | 50% Ethanol | | | |
| 8 | 0.5% Poloxamer 407 + | Clear solution, | Clear solution, | Clear solution, |
| | 10% Propylene glycol + | 52.0 mg/mL | 51.3 mg/mL | 50.4 mg/mL |
| | 50% Ethanol | | | |
| 9 | 2.5% Povidone K30 + | Clear solution, | Clear solution, | Clear solution, |
| | 15% Propylene glycol + | 53.7 mg/mL | 52.6 mg/mL | 50.5 mg/mL |
| | 50% Ethanol | | | |
| 10 | 2.5% Acconon MC-8 + | Clear solution, | Clear solution, | Clear solution, |
| | 15%/Propylene glycol + | 54.7 mg/mL | 53.6 mg/mL | 56.0 mg/mL |
| | 50% | | | |
| | Ethanol | | | |
| 11 | 0.5% Tween80 + 15% | Clear solution, | Clear solution, | Clear solution, |
| | Propylene glycol + | 49.0 mg/mL | 48.0 mg/mL | 47.5 mg/mL |
| | 50% Ethanol | | | |
| 12 | 1% Poloxamer 407 + | Clear solution, | Clear gel-like | Clear solution, |
| | 30% | 40.6 mg/mL | solution, | 53.2 mg/mL |
| | Propylene glycol | | 39.3 mg/mL | |
| 13 | 2.5% Span80 + 10% | Clear solution, | Clear solution, | Clear solution, |
| | Propylene glycol + | 57.4 mg/mL | 56.4 mg/mL | 47.9 mg/mL |
| | 50% Ethanol | | | |
| 14 | 0.5% Span80 + 10% | Clear solution, | Clear solution, | Clear solution, |
| | Propylene glycol + | 53.9 mg/mL | 52.9 mg/mL | 48.9 mg/mL |
| | 50% Ethanol | | | |
| 15 | 1% Poloxamer 407 + | Clear solution, | Clear solution, | Clear solution, |
| | 30% | 52.5 mg/mL | 52.7 mg/mL | 50.8 mg/mL |
| | Propylene glycol + 30% | | | |
| | Ethanol | | | |
| 16 | 1% Tween80 + 1% | Clear gel-like | Clear solution, | Clear solution, |
| | Providone + 30% | solution, | 51.2 mg/mL | 49.5 mg/mL |
| | Propylene glycol + | 49.7 mg/mL | | |
| | 10% Ethanol | | | |
| 17 | 1% Tween80 + 1% | Clear solution, | Clear solution, | Clear solution, |
| | Providone + 30% | 50.6 mg/mL | 51.1 mg/mL | 59.1 mg/mL |
| | Propylene glycol + 20% | | | |
| | Ethanol | | | |
| 18 | 1% Tween80 + 30% | Clear gel-like | Clear gel-like | Gel-like solution, |
| | Propylene glycol + | solution, | solution, | 19.2 mg/mL |
| | 20% Ethanol | 58.7 mg/mL | 57.7 mg/mL | |
| 19 | 1% Providone + 30% | Clear gel-like | Cloudy | Cloudy |
| | Propylene glycol + | solution, | 57.7 mg/mL | N/A |
| | 20% Ethanol | 58.7 mg/mL | | |
| 20 | 1% Poloxamer407 + | Clear solution, | Clear solution, | Clear solution, |
| | 30% Propylene glycol + | 48.5 mg/mL | 47.9 mg/mL | 53.7 mg/mL |
| | 25% Ethanol | | | |
| 21 | 40% Ethanol + 40% | Clear solution, | Clear solution, | Clear solution, |
| | Propylene glycol + | 56.2 mg/mL | 56.2 mg/mL | 51.1 mg/mL |
| | 20% Glycerin | | | |
| 22 | 40% Ethanol + 20% | Clear solution, | Clear solution, | Clear solution, |
| | Propylene glycol + 20% | 49.2 mg/mL | 49.2 mg/mL | 50.1 mg/mL |
| | PEG 400 + 20% | | | |
| | Glycerin | | | |

### Example 19

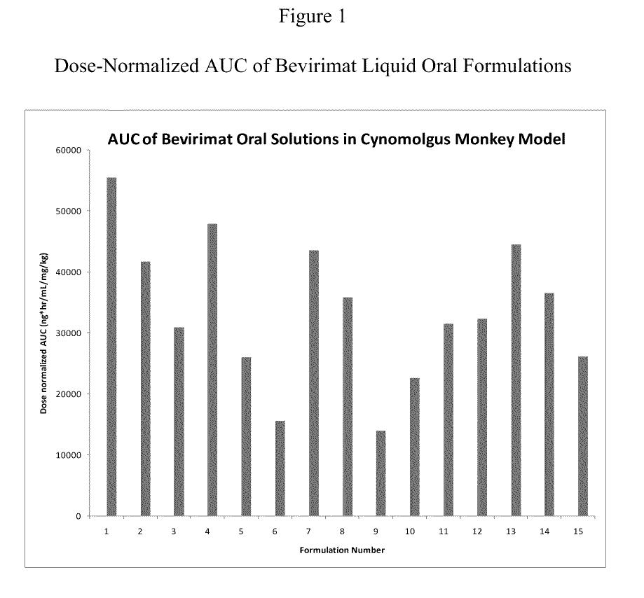

Bevirimat liquid oral formulations 3-15 described in Table 5 were tested in a Cynomolgus monkey model as described in Example 20 below for oral bioequivalency relative to bevirimat dimeglumine administered as an oral solution in 10% hydroxypropyl-β-cyclodextrin (n=21). The results are shown in FIG. 1.

TABLE 5
Composition of Formulations Tested in Cynomologus Monkey (FIG. 1)
and Relative Oral Bioavailabilities
All liquids are expressed as (v/v %). All solids are expressed as (w/v %)
including Poloxamers, Vitamine E TPGS and Povidones

| | | Dose | |
|---|---|---|---|
| | | Normalized | |
| | | AUC | |
| | | (ng * hr/mL/ | Relative Oral |
| Form # | Composition of Test Formulations | mg/kg) | Bioavailability % |
| 1 | HPβCD IV; | 55,466 | N/A |
| | 2.5 mg BVM FAE/mL, 10 mg BVM FAE dose | | |
| | (IV reference dose) (n = 6) | | |
| 2 | HPβCD oral gavage; | 41,603 | 100% |
| | 12.5 mg BVM FAE/mL, 50 mg BVM FAE dose | | |
| | (Oral reference dose) (n = 21) | | |
| 3 | 40% PG/20% Glycerin/1% TPGS/0.25% Tween80; | 30,841 | 74% |
| | 12.5 mg BVM FAE/mL, 50 mg BVM FAE dose | | |
| 4 | 40% PG/20% Glycerin/1% TPGS/0.25% Tween80/ | 47,805 | 115% |
| | 0.2% Povidone K15; | | |
| | 12.5 mg BVM FAE/mL, 50 mg BVM FAE dose | | |
| 5 | 40% PG/20% Glycerin/1% TPGS/0.25% Tween80 | 25,965 | 62% |
| | 0.2% NaCMC; 12.5 mg BVM FAE/mL, | | |
| | 50 mg BVM FAE dose | | |
| 6 | 40% PG/20% Glycerin/0.25% Tween80/ | 15,596 | 37% |
| | 0.2% Povidone K15; 12.5 mg BVM FAE/mL, | | |
| | 50 mg BVM FAE dose | | |
| 7 | 40% PG/20% Glycerin/5% TPGS/0.25% Tween80/ | 43,523 | 105% |
| | 0.2% Povidone K15/10 mM phosphate pH 8; | | |
| | 12.5 mg BVM FAE/mL, 50 mg BVM FAE dose | | |
| 8 | 40% PG/20% Glycerin/5% TPGS/0.25% Tween80/ | 35,761 | 86% |
| | 0.2% Povidone K15/10 mM phosphate pH8; | | |
| | 12.5 mg BVM FAE/mL, 200 mg BVM FAE dose | | |
| 9 | 40% EtOH/40% PG/20% Glycerin; | 13,936 | 33% |
| | 50 mg BVM FAE/mL, 50 mg BVM FAE dose | | |
| 10 | 30% EtOH/30% PG/10% Glycerin/1% TPGS; | 22,629 | 54% |
| | 25 mg BVM FAE/mL, 50 mg BVM FAE dose | | |
| 11 | 15% EtOH/15% PG/5% Glycerin/0.5% TPGS; | 31,447 | 76% |
| | 12.5 mg BVM FAE/mL, 50 mg BVM FAE dose | | |
| 12 | 15% EtOH/15% PG/5% Glycerin/0.5% | 32,327 | 78% |
| | TPGS/0.2% Povidone K90; | | |
| | 12.5 mg BVM FAE/mL, 50 mg BVM FAE dose | | |
| 13 | 15% EtOH/30% PG/10% Glycerin; | 44,447 | 107% |
| | 12.5 mg BVM FAE/mL, 50 mg BVM FAE dose | | |
| 14 | 15% EtOH/30% PG/10% Glycerin/5% TPGS/ | 36,462 | 88% |
| | 10 mM phosphate pH 8; | | |
| | 12.5 mg BVM FAE/mL, 50 mg BVM FAE dose | | |
| 15 | 15% EtOH/30% PG/10% Glycerin/5% TPGS/10 mM | 26,093 | 63% |
| | phosphate pH 8; | | |
| | 12.5 mg BVM FAE/mL, 200 mg BVM FAE dose | | |

Formulations 1 and 2 were reference HPβCD controls for IV and oral routes of administration in the Cynomolgus monkey model and had dose-normalized “areas-under-the-curves” (AUC) of 55,466 and 41,603 ng*hr/mL/mg/kg, respectively, indicating an absolute oral bioavailability of ˜55% for bevirimat administered as a bevirimat-cyclodextrin carrier complex in water. All oral formulations were administered at a dose of 50 mg BVM FAE (i.e., bevirimat free acid equivalents) with the exceptions of Formulations 8 and 15, which were dosed at 200 mg BVM FAE.

Carboxymethylcellulose (CMC), Povidone and D-tocopheryl polyethylene glycol 1000 succinate TPGS (TPGS) were introduced as crystallization inhibitors to improve bevirimat dispersion and prevent precipitation in simulated intestinal fluids and in vivo. These inhibitors may prevent soluble bevirimat from precipitating into a metastable crystalline form in the stomach by impeding nucleation and growth of drug crystals.

It was found that concentrations of at least 5% Tween 80 in the 50 mg/mL bevirimat formulations were necessary to achieve clear, particulate-free mixtures following dispersion in simulated intestinal fluids. However, concentrations as low as 1% (v/v) Tween-80 resulted in clear, particulate-free mixtures with 25 mg/mL bevirimat formulations, indicating the importance of bevirimat concentration in the oral solutions. Additional experimentation indicated that the following low dose bevirimat formulations were found to have good dispersion properties in SGF and SIF: 12.5 mg/mL bevirimat formulated in 40% PG +20% glycerin+1% Tween 80+1% TPGS containing inhibitor (Povidone K15) and 12.5 mg/mL bevirimat formulated in 15% ethanol+10% glycerin+30% PG+1% TPGS. It was found that lowered Tween 80 levels to 0.25% improve palatability and taste.

Formulation 3 (“Tween 80 formulation”) containing 12.5 mg bevirimat FAE/mL, 40% (v/v) propylene glycol, 20% (v/v)glycerin, 1% (v/v) TPGS, 0.25% (v/v) Tween 80 had a dose-normalized AUC of 30,841 ng*hr/mL/mg/kg (relative oral bioavailability ˜74%). Addition of crystallization inhibitors improved the relative bioavailability to ˜115% when Povidone K15 was used as the inhibitor (Formulation 4) but decreased relative bioavailability to 62% when Na CMC was used as the inhibitor (Formulation 5). In Formulation 6, relative oral bioavailability was 37% when Vitamin E TPGS was omitted from the formulation, even when Povidone K15 was present at 0.2% (v/v). Formulation 7 contained 5% Vitamin E TPGS and 0.2% Povidone K15 as crystallization inhibitors, was buffered to pH 8 with 10 mM sodium phosphate buffer and had a relative oral bioavailability of 105%. Formulation 7 was dosed at 200 mg (i.e., Formulation 8) to evaluate dose-dependency and the relative oral bioavailability of Formulation 8 was only slightly reduced to 86% at the higher dose level.

Formulation 9 (“Ethanol formulation”) contained 50 mg bevirimat FAE/mL, 40% (v/v) ethanol, 40% (v/v) propylene glycol, 20% (v/v) glycerin and had a dose-normalized AUC of 13,936 ng*hr/mL/mg/kg (relative oral bioavailability ˜33%). For Formulation 10, solvent levels were reduced to improve taste and palatability and Vitamin E TPGS was added to inhibit crystallization and to solubilize bevirimat. The composition of Formulation 10 was 25 mg bevirimat FAE/mL, 30% (v/v) ethanol, 30% (v/v) propylene glycol, 10% (v/v) glycerin and 1% (v/v) Vitamin E TPGS. The relative oral bioavailability of this formulation increased to 54%. Formulation 10 was diluted 1:1 with water to produce Formulation 11 and the relative bioavailability increased again from 54% to 76%, indicating that reducing the concentration of bevirimat in the oral solution might play a role in increasing absorption, perhaps by reducing precipitation in situ. Formulation 12 was similar to Formulation 7 except for the addition of 0.2% Povidone K90 as a crystallization inhibitor and relative oral bioavailability was unchanged at 78%. Povidone did not enhance bioavailability in “ethanol-based” bevirimat oral solutions. In Formulation 13, composed of 12.5 mg bevirimat FAE/mL in 15% (v/v) EtOH/30% (v/v), propylene glycol/10% (v/v) glycerin, the relative oral bioavailability was 107% when Vitamin E TPGS was omitted from the formulation, indicating that addition of bevirimat precipitation inhibitors, either Vitamin E TPGS or Povidone K15, was not necessary at 12.5 mg bevirimat/mL dose levels. To confirm this, 5% Vitamin E TPGS was added to Formulation 13 and the oral solution was buffered to pH 8 with 10 mM sodium phosphate buffer to produce Formulation 14. The relative oral bioavailability of Formulation 14 was reduced to 88% from 105%. Formulation 14 was dosed at 200 mg (i.e., Formulation 15) to evaluate dose-dependency and the relative oral bioavailability of Formulation 14 was reduced from 88% to 63% at the higher dose level. Vitamin E TPGS had a negative effect on the bioavailability of the “Ethanol formulation”, but not on the bioavailability of the “Tween-80 formulation”.

### Example 20

### Pharmacokinetic Evaluation of Liquid Bevirimat Dosage Forms

To evaluate the pharmacokinetic performance of certain bevirimat liquid oral dosage forms, various formulations were administered to Cynomolgus monkeys according to the following parameters.

Six male Cynomolgus monkeys having an initial body weight of about 3.5 to about 4.5 kg are allowed 7 days to acclimate to their individual housing units. Monkeys are supplied with water and a commercial diet ad libitum prior to the study initiation. Food is withheld from the monkeys for a minimum of twelve hours before the study and during the study until four hours postdose when food is returned. Water is supplied ad libitum.

Test formulations are administered to the monkeys at time 0 on appropriate days. Test formulations will be dosed orally via oral gavage or intravenously.

Samples are drawn at the frequencies indicated below. Each blood sample (0.5 mL) is collected from the monkey's saphenous vein via a butterfly catheter and placed into a chilled polypropylene tube containing sodium heparin as the anticoagulant. Blood samples are centrifuged at a temperature of 4° C. at a speed of 3,000 rpm for 15 minutes. All samples are maintained chilled throughout processing. Plasma is collected after centrifugation, pipetted into a 2-mL polypropylene microtube, and frozen on dry-ice pending analytical evaluation.

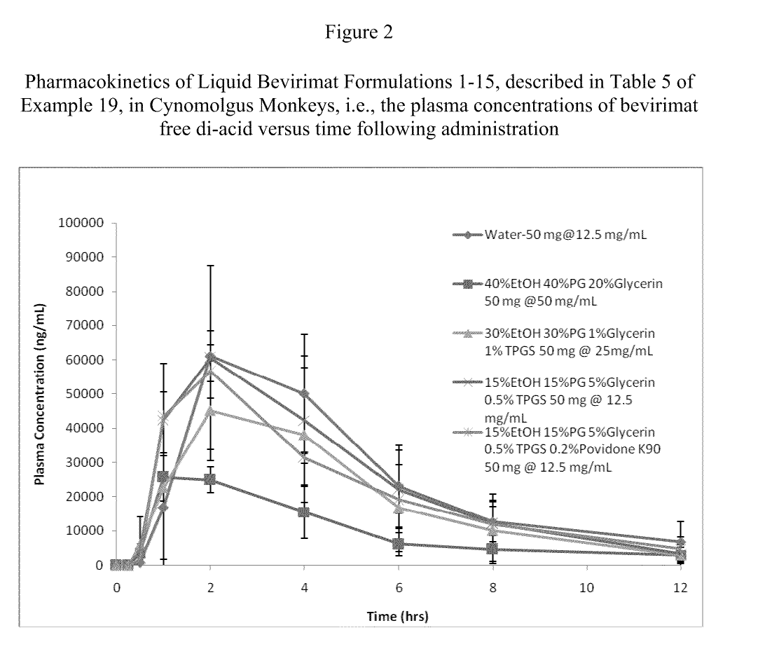

Pharmacokinetics of exemplary liquid bevirimat formulations in Cynomolgus monkeys, i.e., the plasma concentrations of bevirimat free di-acid versus time following administration, are shown in FIG. 2.

### Example 21

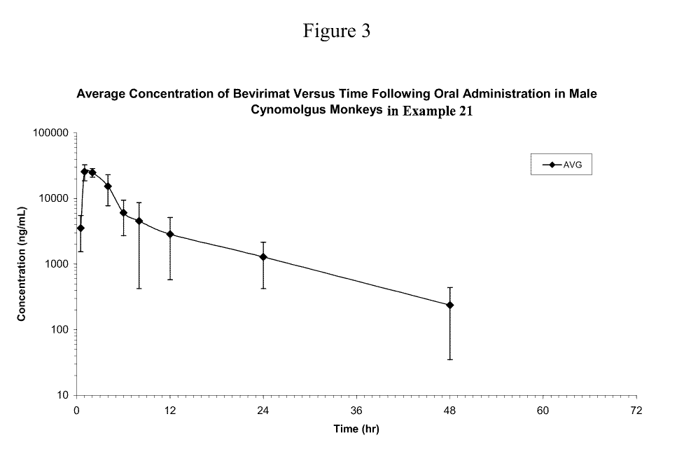

Male Cynomolgus monkeys were administered a bevirimat dimeglumine liquid formulation comprising 50 mg bevirimat free acid equivalents/mL in 40% EtOH: 40% PG: 20% Glycerol, 50 mg bevirimat free acid equivalents/Monkey by oral gavage. Results are shown below in Table 6. FIG. 3 is a graph depicting the average plasma concentration of bevirimat versus time.

TABLE 6
Individual and Average Plasma Concentrations (ng/mL) for
Bevirimat Following Oral Administration to Male Cynomolgus
Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | BLOQ | 75.5 | BLOQ | ND |
| 0.50 | 1270 | 4350 | 5020 | 3547 |
| 1.0 | 19200 | 24900 | 33100 | 25733 |
| 2.0 | 25800 | 20800 | 28100 | 24900 |
| 4.0 | 22700 | 7410 | 16200 | 15437 |
| 6.0 | 10000 | 4250 | 4040 | 6097 |
| 8.0 | 9330 | 2570 | 1800 | 4567 |
| 12 | 2500 | 785 | 5310 | 2865 |
| 24 | 2290 | 762 | 816 | 1289 |
| 48 | 461 | 65.1 | 187 | 238 |
| 72 | 76.0 | BLOQ | BLOQ | ND |
| Animal Weight (kg) | 4.5 | 3.7 | 4.1 | 4.1 |
| Dose (mg/kg) | 11.1 | 13.5 | 12.2 | 12.3 |
| Cₘₐₓ (ng/mL) | 25800 | 24900 | 33100 | 27933 |
| tₘₐₓ (hr) | 2.0 | 1.0 | 1.0 | 1.3 |
| t_{1/2} (hr) | 9.8 | 7.8 | 7.8 | 8.5 |
| AUCₐₗₗ (hr * ng/mL) | 220321 | 103332 | 174777 | 166143 |
| Dose Normalized Values | | | | |
| D_{nzed} AUCₐₗₗ | 19829 | 7647 | 14332 | 13936 |
| (hr * ng/mL/mg/kg) | | | | |

### Example 22

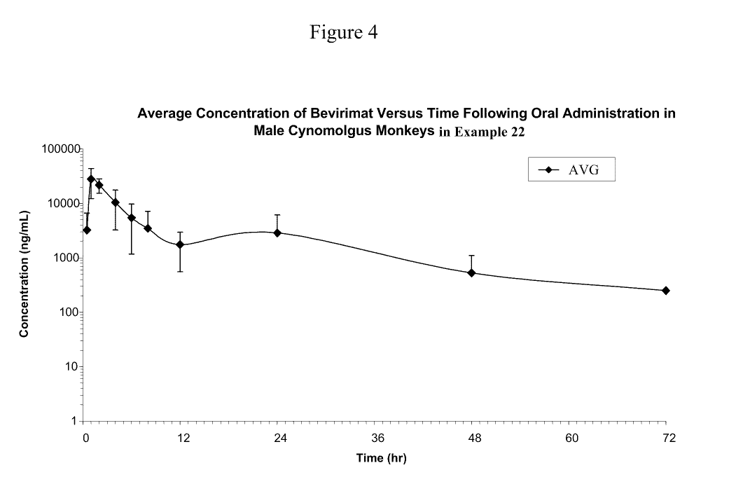

Male Cynomolgus monkeys were administered a bevirimat dimeglumine liquid formulation comprising 50 mg bevirimat free acid equivalents/mL in 40% PG: 20% Glycerin: 1% TPGS: 5% Tween 80 in SWFI, 50 mg bevirimat free acid equivalents/Monkey by oral gavage. Results are shown below in Table 7. FIG. 4 is a graph depicting the average plasma concentration of bevirimat versus time.

TABLE 7
Individual and Average Plasma Concentrations (ng/mL) for
Bevirimat Following Oral Administration to Male Cynomolgus
Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | BLOQ | BLOQ | 2350 | ND |
| 0.50 | 709 | 1960 | 7090 | 3253 |
| 1.0 | 9910 | 38700 | 35700 | 28103 |
| 2.0 | 17900 | 29200 | 18400 | 21833 |
| 4.0 | 18400 | 8760 | 4260 | 10473 |
| 6.0 | 10300 | 3640 | 2370 | 5437 |
| 8.0 | 7710 | 1600 | 1150 | 3487 |
| 12 | 3010 | 602 | 1680 | 1764 |
| 24 | 6670 | 1040 | 934 | 2881 |
| 48 | 1200 | 163 | 227 | 530 |
| 72 | 398 | BLOQ | 105 | 252 |
| Animal Weight (kg) | 4.7 | 3.8 | 4.2 | 4.2 |
| Dose (mg/kg) | 10.6 | 13.2 | 11.9 | 11.9 |
| Cₘₐₓ (ng/mL) | 18400 | 38700 | 35700 | 30933 |
| tₘₐₓ (hr) | 4.0 | 1.0 | 1.0 | 2.0 |
| t_{1/2} (hr) | 11.8 | 11.8 | 14.6 | 12.8 |
| AUCₐₗₗ (hr * ng/mL) | 292883 | 128897 | 111291 | 177690 |
| Dose Normalized Values | | | | |
| D_{nzed} AUCₐₗₗ | 27531 | 9796 | 9348 | 15559 |
| (hr * ng/mL/mg/kg) | | | | |

### Example 23

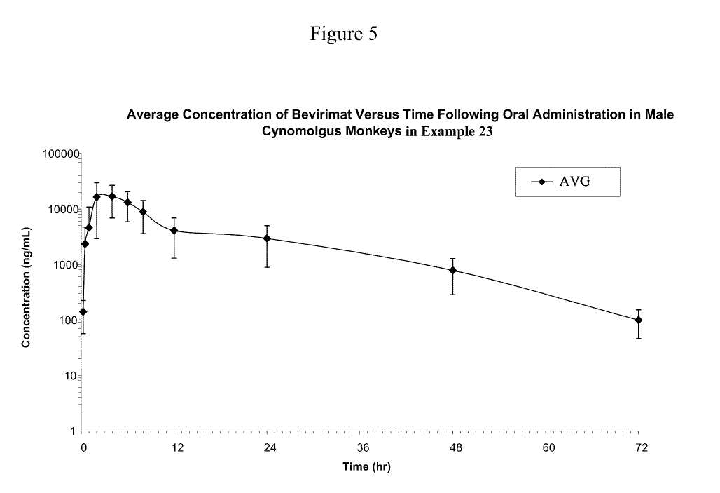

Male Cynomolgus monkeys were administered a bevirimat dimeglumine liquid formulation comprising 10% HPβCD, 50 mg bevirimat free acid equivalents/Monkey by oral gavage. Results are shown below in Table 8. FIG. 5 is a graph depicting the average plasma concentration of bevirimat versus time.

TABLE 8
Individual and Average Plasma Concentrations (ng/mL) for Bevirimat
Following Oral Administration to Male Cynomolgus Monkeys

| | Monkeys # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | 75.0 | 112 | 235 | 141 |
| 0.50 | 5030 | 1440 | 563 | 2344 |
| 1.0 | 958 | 11800 | 1040 | 4599 |
| 2.0 | 25900 | 22300 | 977 | 16392 |
| 4.0 | 24900 | 19800 | 5740 | 16813 |
| 6.0 | 19700 | 14600 | 5300 | 13200 |
| 8.0 | 14600 | 7960 | 4090 | 8883 |
| 12 | 7320 | 2350 | 2620 | 4097 |
| 24 | 5310 | 1750 | 1770 | 2943 |
| 48 | 1350 | 579 | 417 | 782 |
| 72 | 158 | 54.0 | 86.0 | 99.3 |
| Animal Weight (kg) | 4.7 | 3.8 | 4.1 | 4.2 |
| Dose (mg/kg) | 10.6 | 13.2 | 12.2 | 12.0 |
| Cₘₐₓ (ng/mL) | 25900 | 22300 | 5740 | 17980 |
| tₘₐₓ (hr) | 2.0 | 2.0 | 4.0 | 2.7 |
| t_{1/2} (hr) | 10.0 | 10.2 | 11.0 | 10.4 |
| AUCₐₗₗ (hr * ng/mL) | 362910 | 200392 | 100725 | 221342 |
| Dose Normalized Values | | | | |
| D_{nzed} AUCₐₗₗ | 34113 | 15230 | 8259 | 19201 |
| (hr * ng/mL/mg/kg) | | | | |

### Example 24

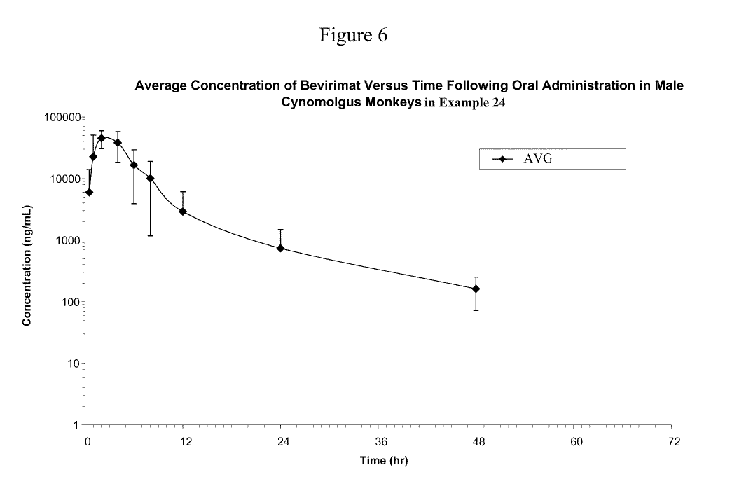

Male Cynomolgus monkeys were administered a bevirimat dimeglumine liquid formulation comprising 25 mg/mL bevirimat in 30% EtOH: 1% TPGS: 30% PG: 10% Glycerin, 50 mg bevirimat free acid equivalents/Monkey by oral gavage. Results are shown below in Table 9. FIG. 6 is a graph depicting the average plasma concentration of bevirimat versus time.

TABLE 9
Individual and Average Plasma Concentrations (ng/mL) for
bevirimat Following Oral Administration to Male Cynomolgus
Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | 147 | BLOQ | BLOQ | ND |
| 0.50 | 2680 | 85.2 | 15200 | 5988 |
| 1.0 | 11400 | 1940 | 54500 | 22613 |
| 2.0 | 56700 | 28700 | 50100 | 45167 |
| 4.0 | 60300 | 22900 | 31000 | 38067 |
| 6.0 | 30900 | 6420 | 12600 | 16640 |
| 8.0 | 20000 | 2790 | 7430 | 10073 |
| 12 | 6550 | 575 | 1640 | 2922 |
| 24 | 1600 | 367 | 256 | 741 |
| 48 | 261 | 142 | 84.5 | 163 |
| 72 | 69.3 | BLOQ | BLOQ | ND |
| Animal Weight (kg) | 4.5 | 3.8 | 4.0 | 4.1 |
| Dose (mg/kg) | 11.1 | 13.2 | 12.5 | 12.3 |
| Cₘₐₓ (ng/mL) | 60300 | 28700 | 54500 | 47833 |
| tₘₐₓ (hr) | 4.0 | 2.0 | 1.0 | 2.3 |
| t_{1/2} (hr) | 10.6 | 17.8 | 9.0 | 12.5 |
| AUCₐₗₗ (hr * ng/mL) | 425337 | 124468 | 251857 | 267221 |
| Dose Normalized Values | | | | |
| D_{nzed} AUCₐₗₗ | 38280 | 9460 | 20149 | 22629 |
| (hr * ng/mL/mg/kg) | | | | |

### Example 25

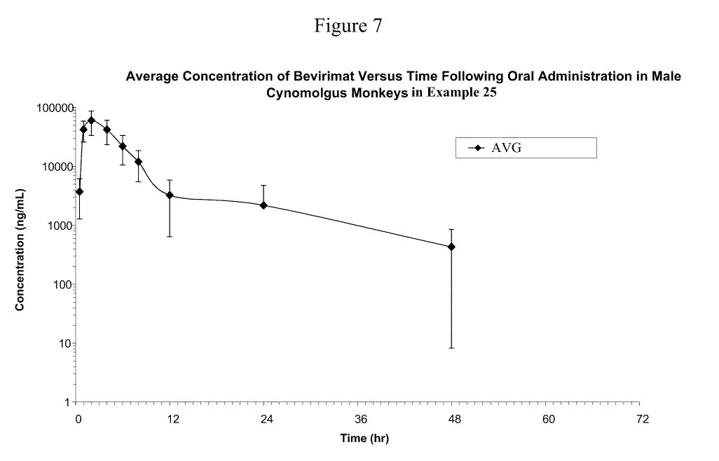

Male Cynomolgus monkeys were administered a bevirimat dimeglumine liquid formulation comprising 12.5 mg bevirimat free acid equivalents/mL in 15% EtOH: 0.5% TPGS: 15% PG: 5% Glycerin, 50 mg bevirimat free acid equivalents/Monkey by oral gavage. Results are shown below in Table 10. FIG. 7 is a graph depicting the average plasma concentration of bevirimat versus time.

TABLE 10
Individual and Average Plasma Concentrations (ng/mL) for
Bevirimat Following Oral Administration to Male Cynomolgus
Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | 114 | BLOQ | BLOQ | ND |
| 0.50 | 5060 | 5220 | 909 | 3730 |
| 1.0 | 60800 | 37600 | 28900 | 42433 |
| 2.0 | 73300 | 78800 | 29700 | 60600 |
| 4.0 | 57000 | 48700 | 21100 | 42267 |
| 6.0 | 33300 | 22500 | 10400 | 22067 |
| 8.0 | 19200 | 10700 | 6340 | 12080 |
| 12 | 6240 | 2230 | 1320 | 3263 |
| 24 | 5180 | 774 | 631 | 2195 |
| 48 | 922 | 229 | 149 | 433 |
| 72 | 233 | BLOQ | BLOQ | ND |
| Animal Weight (kg) | 4.7 | 3.8 | 4.1 | 4.2 |
| Dose (mg/kg) | 10.6 | 13.2 | 12.2 | 12.0 |
| Cₘₐₓ (ng/mL) | 73300 | 78800 | 29700 | 60600 |
| tₘₐₓ (hr) | 2.0 | 2.0 | 2.0 | 2.0 |
| t_{1/2} (hr) | 12.0 | 11.3 | 11.5 | 11.6 |
| AUCₐₗₗ (hr * ng/mL) | 563760 | 358030 | 172406 | 364732 |
| Dose Normalized Values | | | | |
| D_{nzed} AUCₐₗₗ | 52993 | 27210 | 14137 | 31447 |
| (hr * ng/mL/mg/kg) | | | | |

### Example 26

Male Cynomolgus monkeys were administered a bevirimat dimeglumine liquid formulation comprising 2.5 mg bevirimat free acid equivalents/mL in 10% HPβCD+3% Mannitol in water, 10 mg bevirimat free acid equivalents/Monkey by intravenous injection. Results are shown below in Table 11.

TABLE 11
Individual and Average Plasma Concentrations (ng/mL) for
bevirimat Following Oral Administration to Male Cynomolgus
Monkeys

| | Monkey # | | | |
|---|---|---|---|---|
| | 115 | 116 | 120 | AVG |
| 3/8 Time (hr) | | | | |
| 0 | BLOQ | BLOQ | BLOQ | ND |
| (pre-dose) | | | | |
| 0.033 | 50600 | 51700 | 39400 | 47233 |
| 0.083 | NS | 47500 | 49200 | 48350 |
| 0.25 | 32300 | 41600 | 23100 | 32333 |
| 0.50 | 25300 | 19800 | 14800 | 19967 |
| 1.0 | 22800 | 20200 | 11700 | 18233 |
| 2.0 | 17700 | 8810 | 6750 | 11087 |
| 4.0 | 8260 | 5430 | 3540 | 5743 |
| 6.0 | 4430 | 1470 | 1580 | 2493 |
| 8.0 | 2370 | 1210 | 866 | 1482 |
| 12 | 762 | 384 | 404 | 517 |
| 24 | 347 | 367 | 245 | 320 |
| 48 | 134 | 148 | 81.0 | 121 |
| 72 | BLOQ | BLOQ | BLOQ | ND |
| Animal Weight (kg) | 4.1 | 5.1 | 5.4 | 4.9 |
| Dose (mg/kg) | 2.4 | 2.0 | 1.9 | 2.1 |
| C₀ (ng/mL)¹ | 54175 | 54673 | 39400 | 49416 |
| tₘₐₓ (h)¹ | 0 | 0 | 0 | 0 |
| t_{1/2} (hr) | 14.7 | 24.7 | 15.5 | 18.3 |
| CL (L/hr/kg) | 0.020 | 0.023 | 0.032 | 0.025 |
| Vₛₛ (L/kg) | 0.13 | 0.23 | 0.22 | 0.19 |
| AUCₐₗₗ (hr * ng/mL) | 114338 | 81549 | 58342 | 84743 |
| Dose Normalized | | | | |
| Values | | | | |
| D_{nzed} AUCₐₗₗ | 46879 | 41590 | 31505 | 39991 |
| (hr * ng/mL/mg/kg) | | | | |

### Example 27

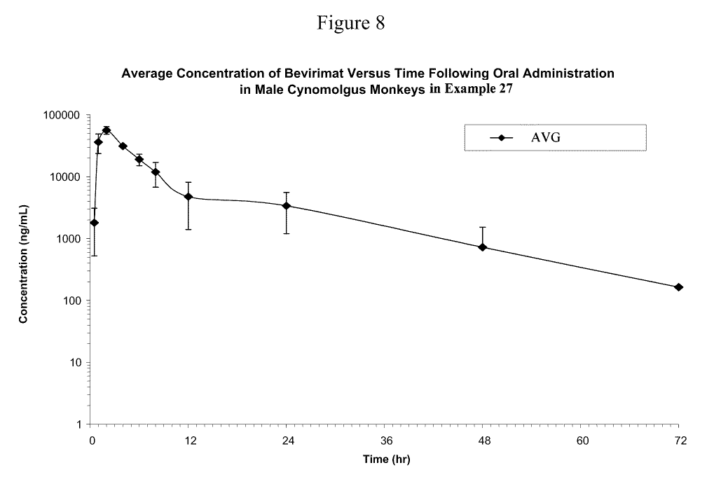

Male Cynomolgus monkeys were administered a bevirimat dimeglumine liquid formulation comprising 12.5 mg bevirimat free acid equivalents/mL in 15% EtOH: 0.5% TPGS: 15% PG: 5% Glycerin, 0.2% Povidone K90, 50 mg bevirimat free acid equivalents/Monkey by oral gavage. Results are shown below in Table 12. FIG. 8 is a graph depicting the average plasma concentration of bevirimat versus time.

TABLE 12
Individual and Average Plasma Concentrations (ng/mL) for
Bevirimat Following Oral Administration to Male Cynomolgus
Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | BLOQ | BLOQ | BLOQ | ND |
| 0.50 | 2940 | 412 | 2080 | 1811 |
| 1.0 | 46300 | 22100 | 40700 | 36367 |
| 2.0 | 51300 | 65600 | 52800 | 56567 |
| 4.0 | 33100 | 30800 | 30100 | 31333 |
| 6.0 | 23100 | 15000 | 19300 | 19133 |
| 8.0 | 17000 | 6720 | 12000 | 11907 |
| 12 | 8210 | 1510 | 4560 | 4760 |
| 24 | 5710 | 1340 | 3140 | 3397 |
| 48 | 1650 | 296 | 238 | 728 |
| 72 | 277 | BLOQ | 51.0 | 164 |
| Animal Weight (kg) | 4.8 | 3.8 | 4.3 | 4.3 |
| Dose (mg/kg) | 10.4 | 13.2 | 11.6 | 11.7 |
| Cₘₐₓ (ng/mL) | 51300 | 65600 | 52800 | 56567 |
| tₘₐₓ (hr) | 2.0 | 2.0 | 2.0 | 2.0 |
| t_{1/2} (hr) | 12.2 | 14.5 | 8.7 | 11.8 |
| AUCₐₗₗ (hr * ng/mL) | 487562 | 270194 | 344629 | 367461 |
| Dose Normalized Values | | | | |
| D_{nzed} AUCₐₗₗ | 46806 | 20535 | 29638 | 32326 |
| (hr * ng/mL/mg/kg) | | | | |

### Example 28

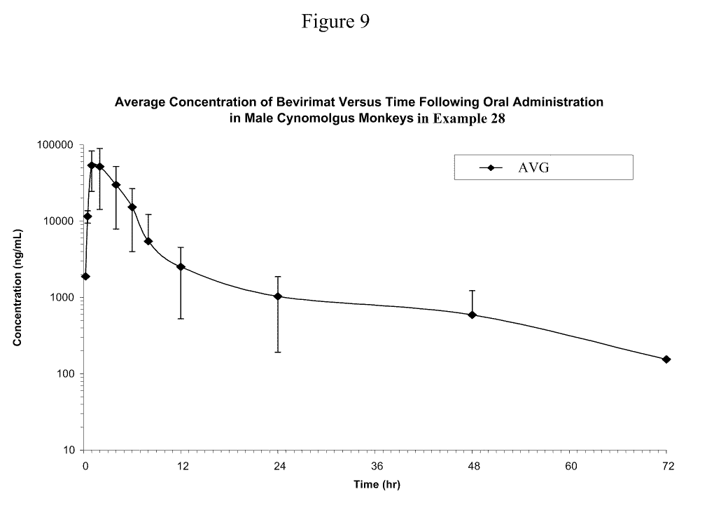

Male Cynomolgus monkeys were administered a bevirimat dimeglumine liquid formulation comprising 12.5 mg bevirimat free acid equivalents/mL in 40% PG, 20% Glycerin, 1% TPGS, 0.25% Tween80, 0.2% NaCMC, 50 mg bevirimat free acid equivalents/Monkey by oral gavage. Results are shown below in Table 13. FIG. 9 is a graph depicting the average plasma concentration of bevirimat versus time.

TABLE 13
Individual and Average Plasma Concentrations (ng/mL) for
Bevirimat Following Oral Administration to Male Cynomolgus
Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | 246 | 3540 | BLOQ | 1893 |
| 0.50 | 12500 | 13100 | 9140 | 11580 |
| 1.0 | 52800 | 25100 | 83600 | 53833 |
| 2.0 | 49100 | 15800 | 91100 | 52000 |
| 4.0 | 35300 | 5720 | 48900 | 29973 |
| 6.0 | 18300 | 2820 | 24900 | 15340 |
| 8.0 | 13300 | 1830 | 1280 | 5470 |
| 12 | 4470 | 467 | 2660 | 2532 |
| 24 | 1980 | 357 | 770 | 1036 |
| 48 | 1330 | 169 | 282 | 594 |
| 72 | 252 | BLOQ | 58.6 | 155 |
| Animal Weight (kg) | 4.9 | 3.9 | 4.3 | 4.4 |
| Dose (mg/kg) | 10.2 | 12.8 | 11.6 | 11.6 |
| Cₘₐₓ (ng/mL) | 52800 | 25100 | 91100 | 56333 |
| tₘₐₓ (hr) | 1.0 | 1.0 | 2.0 | 1.3 |
| t_{1/2} (hr) | 15.7 | 24.2 | 11.5 | 17.2 |
| AUCₐₗₗ (hr * ng/mL) | 371443 | 85111 | 396829 | 284461 |
| Dose Normalized Values | | | | |
| D_{nzed} AUCₐₗₗ | 36401 | 6639 | 34127 | 25722 |
| (hr * ng/mL/mg/kg) | | | | |

### Example 29

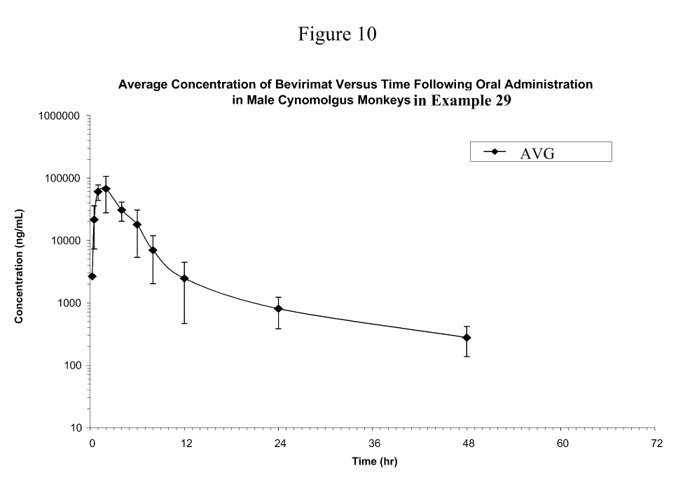

Male Cynomolgus monkeys were administered a bevirimat dimeglumine liquid formulation comprising 12.5 mg bevirimat free acid equivalents/mL in 40% PG, 20% Glycerin, 1% TPGS, 0.25% Tween80, 50 mg bevirimat free acid equivalents/Monkey by oral gavage. Results are shown below in Table 14. FIG. 10 is a graph depicting the average plasma concentration of bevirimat versus time.

TABLE 14
Individual and Average Plasma Concentrations (ng/mL) for bevirimat
Following Oral Administration to Male Cynomolgus Monkeys

| | Monkey # | | | |
|---|---|---|---|---|
| | 186 | 116 | 120 | AVG |
| Time (hr) | | | | |
| 0 | BLOQ | BLOQ | BLOQ | ND |
| (pre-dose) | | | | |
| 0.25 | 1640 | 3670 | BLOQ | 2655 |
| 0.50 | 28500 | 30900 | 5160 | 21520 |
| 1.0 | 65500 | 74400 | 41900 | 60600 |
| 2.0 | 47700 | 113000 | 41300 | 67333 |
| 4.0 | 34000 | 39100 | 19100 | 30733 |
| 6.0 | 19300 | 29900 | 4740 | 17980 |
| 8.0 | 5470 | 12500 | 2960 | 6977 |
| 12 | 1540 | 4760 | 1100 | 2467 |
| 24 | 1010 | 1090 | 321 | 807 |
| 48 | 135 | 414 | 282 | 277 |
| 72 | BLOQ | 51.8 | BLOQ | ND |
| Animal Weight (kg) | 4.4 | 5.1 | 5.2 | 4.9 |
| Dose (mg/kg) | 11.4 | 9.8 | 9.6 | 10.3 |
| Cₘₐₓ (ng/mL) | 65500 | 113000 | 41900 | 73467 |
| tₘₐₓ (hr) | 1.0 | 2.0 | 1.0 | 1.3 |
| t_{1/2} (hr) | 9.9 | 9.9 | 11.1 | 10.3 |
| AUCₐₗₗ (hr * ng/mL) | 288523 | 481563 | 173216 | 314434 |
| Dose Normalized Values | | | | |
| D_{nzed} AUCₐₗₗ | 25390 | 49119 | 18014 | 30841 |
| (hr * ng/mL/mg/kg) | | | | |

### Example 30

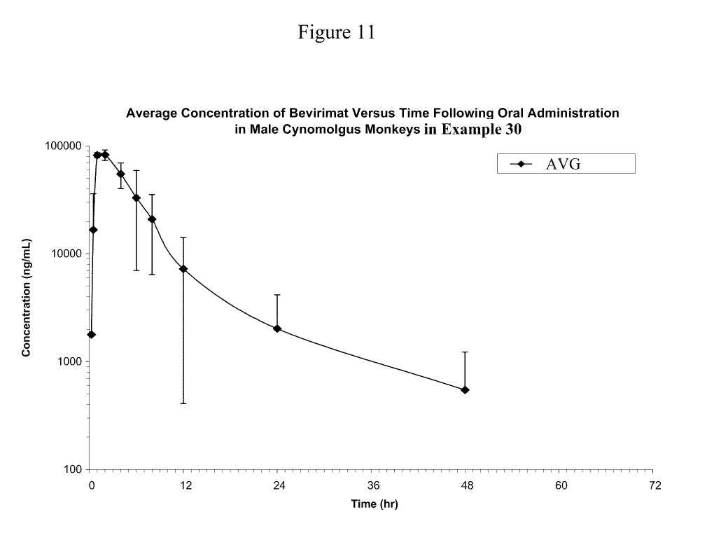

Male Cynomolgus monkeys were administered a bevirimat dimeglumine liquid formulation comprising 12.5 mg bevirimat free acid equivalents/mL in 40% PG, 20% Glycerin, 1% TPGS, 0.25% Tween80, 0.2% Povidone K15, 50 mg bevirimat free acid equivalents/Monkey by oral gavage. Results are shown below in Table 15. FIG. 11 is a graph depicting the average plasma concentration of bevirimat versus time.

TABLE 15
Individual and Average Plasma Concentrations (ng/mL) for
Bevirimat Following Oral Administration to Male Cynomolgus
Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | 2620 | 954 | BLOQ | 1787 |
| 0.50 | 39000 | 8080 | 3150 | 16743 |
| 1.0 | 85200 | 84800 | 77200 | 82400 |
| 2.0 | 93300 | 80200 | 75000 | 82833 |
| 4.0 | 62700 | 64600 | 38000 | 55100 |
| 6.0 | 62600 | 24200 | 12700 | 33167 |
| 8.0 | 36300 | 19300 | 7300 | 20967 |
| 12 | 15000 | 4970 | 1860 | 7277 |
| 24 | 4460 | 1290 | 336 | 2029 |
| 48 | 1340 | 196 | 111 | 549 |
| 72 | 225 | BLOQ | BLOQ | ND |
| Animal Weight (kg) | 4.8 | 3.8 | 4.4 | 4.3 |
| Dose (mg/kg) | 10.4 | 13.2 | 11.4 | 11.6 |
| Cₘₐₓ (ng/mL) | 93300 | 84800 | 77200 | 85100 |
| tₘₐₓ (hr) | 2.0 | 1.0 | 1.0 | 1.3 |
| t_{1/2} (hr) | 10.4 | 7.9 | 6.4 | 8.2 |
| AUCₐₗₗ (hr * ng/mL) | 813770 | 490353 | 318473 | 540865 |
| Dose Normalized Values | | | | |
| D_{nzed} AUCₐₗₗ | 78123 | 37267 | 28026 | 47805 |
| (hr * ng/mL/mg/kg) | | | | |

### Example 31

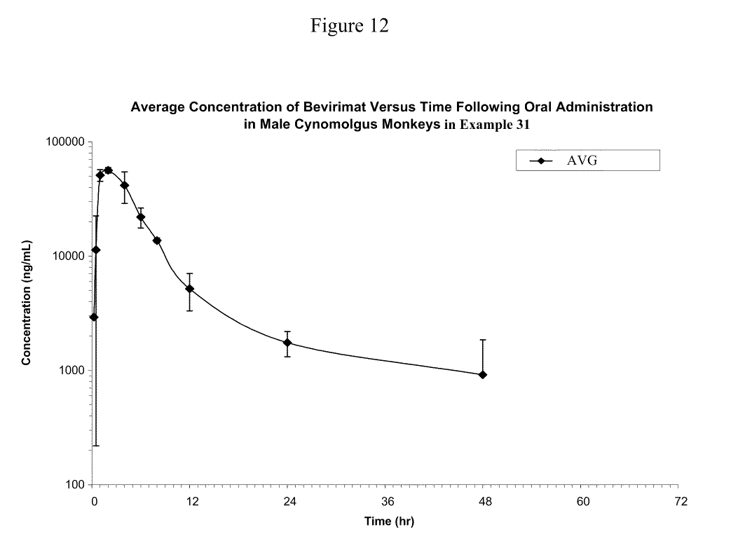

Male Cynomolgus monkeys were administered a bevirimat dimeglumine liquid formulation comprising 12.5 mg bevirimat free acid equivalents/mL in 10% HPβCD, 50 mg bevirimat free acid equivalents/Monkey by oral gavage. Results are shown below in Table 16. FIG. 12 is a graph depicting the average plasma concentration of bevirimat versus time.

TABLE 16
Individual and Average Plasma Concentrations (ng/mL) for Bevirimat
Following Oral Administration to Male Cynomolgus Monkeys

| | Monkey # | | | |
|---|---|---|---|---|
| | 186 | 116 | 120 | AVG |
| Time (hr) | | | | |
| 0 | BLOQ | BLOQ | BLOQ | ND |
| (pre-dose) | | | | |
| 0.25 | 1380 | 399 | 7020 | 2933 |
| 0.50 | 6120 | 3820 | 24200 | 11380 |
| 1.0 | 56400 | 44500 | 52700 | 51200 |
| 2.0 | 59200 | 53000 | 57000 | 56400 |
| 4.0 | 35000 | 33600 | 56500 | 41700 |
| 6.0 | 20700 | 18500 | 26900 | 22033 |
| 8.0 | 13000 | 13900 | 14300 | 13733 |
| 12 | 3140 | 6790 | 5640 | 5190 |
| 24 | 1560 | 1450 | 2250 | 1753 |
| 48 | 2000 | 289 | 467 | 919 |
| 72 | BLOQ | BLOQ | 120 | ND |
| Animal Weight (kg) | 4.4 | 5.2 | 5.0 | 4.9 |
| Dose (mg/kg) | 11.4 | 9.6 | 10.0 | 10.3 |
| Cₘₐₓ (ng/mL) | 59200 | 53000 | 57000 | 56400 |
| tₘₐₓ (hr) | 2.0 | 2.0 | 2.0 | 2.0 |
| t_{1/2} (hr) | 9.9 | 7.0 | 11.4 | 9.4 |
| AUCₐₗₗ (hr * ng/mL) | 385340 | 347663 | 443823 | 392275 |
| Dose Normalized Values | | | | |
| D_{nzed} AUCₐₗₗ | 33910 | 36157 | 44382 | 38150 |
| (hr * ng/mL/mg/kg) | | | | |

### Example 32

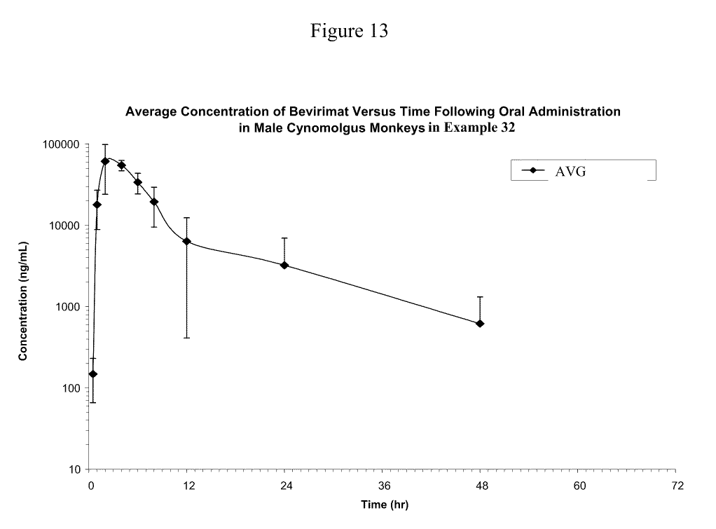

Male Cynomolgus monkeys were administered a bevirimat dimeglumine liquid formulation comprising 12.5 mg bevirimat free acid equivalents/mL in 15% Ethanol, 30% PG, and 10% Glycerin, containing 50 mg bevirimat free acid equivalents, by oral gavage. Results are shown below in Table 17. FIG. 13 is a graph depicting the average plasma concentration of bevirimat versus time.

TABLE 17
Individual and Average Plasma Concentrations (ng/mL) for Bevirimat
Following Oral Administration to Male Cynomolgus Monkeys

| | Monkey # | | | |
|---|---|---|---|---|
| | 117 | 118 | 119 | AVG |
| Time (hr) | | | | |
| 0 | BLOQ | BLOQ | BLOQ | ND |
| (pre-dose) | | | | |
| 0.25 | 66.0 | BLOQ | BLOQ | ND |
| 0.50 | 239 | 75.7 | 132 | 149 |
| 1.0 | 16900 | 9510 | 27600 | 18003 |
| 2.0 | 64100 | 22900 | 97400 | 61467 |
| 4.0 | 46000 | 57200 | 61600 | 54933 |
| 6.0 | 44700 | 26100 | 31200 | 34000 |
| 8.0 | 31000 | 12800 | 14800 | 19533 |
| 12 | 13300 | 2680 | 3210 | 6397 |
| 24 | 7570 | 1110 | 1030 | 3237 |
| 48 | 1430 | 239 | 194 | 621 |
| 72 | 179 | BLOQ | BLOQ | ND |
| Animal Weight (kg) | 4.8 | 3.8 | 4.3 | 4.3 |
| Dose (mg/kg) | 10.4 | 13.2 | 11.6 | 11.7 |
| Cₘₐₓ (ng/mL) | 64100 | 57200 | 97400 | 72900 |
| tₘₐₓ (hr) | 2.0 | 4.0 | 2.0 | 2.7 |
| t_{1/2} (hr) | 8.9 | 10.4 | 9.0 | 9.4 |
| AUCₐₗₗ (hr * ng/mL) | 662459 | 293667 | 445726 | 467284 |
| Dose Normalized Values | | | | |
| D_{nzed} AUCₐₗₗ | 63596 | 22319 | 38332 | 41416 |
| (hr * ng/mL/mg/kg) | | | | |

### Example 33

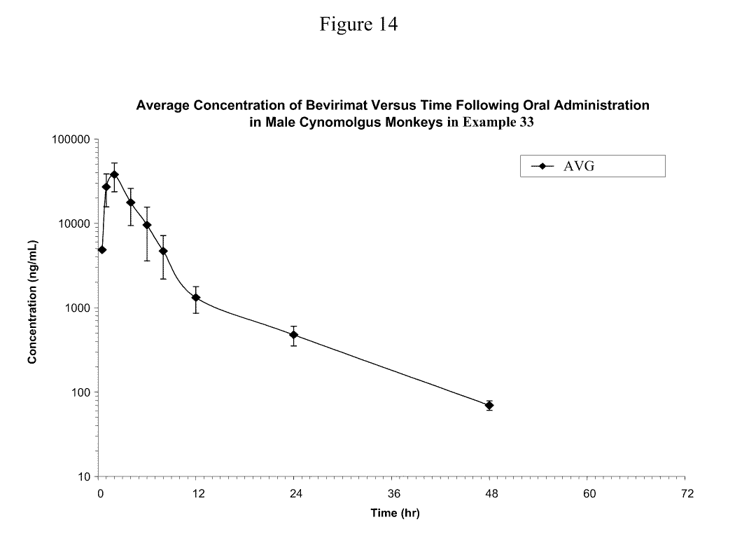

Male Cynomolgus monkeys were administered a bevirimat dimeglumine liquid formulation comprising 12.5 mg bevirimat free acid equivalents/mL in 40% PG, 20% Glycerin, 0.25% Tween80 and 0.2% Povidone K15, containing 50 mg bevirimat free acid equivalents, by oral gavage. Results are shown below in Table 18. FIG. 14 is a graph depicting the average plasma concentration of bevirimat versus time.

TABLE 18
Individual and Average Plasma Concentrations (ng/mL) for Bevirimat
Following Oral Administration to Male Cynomolgus Monkeys

| | Monkey # | | | |
|---|---|---|---|---|
| | 186 | 116 | 120 | AVG |
| Time (hr) | | | | |
| 0 | BLOQ | BLOQ | BLOQ | ND |
| (pre-dose) | | | | |
| 0.25 | BLOQ | 3060 | BLOQ | ND |
| 0.50 | 252 | 9490 | BLOQ | 4871 |
| 1.0 | 36900 | 30000 | 14600 | 27167 |
| 2.0 | 42800 | 49100 | 22000 | 37967 |
| 4.0 | 21400 | 23500 | 8220 | 17707 |
| 6.0 | 15200 | 10300 | 3270 | 9590 |
| 8.0 | 6620 | 5590 | 1870 | 4693 |
| 12 | 1430 | 1720 | 819 | 1323 |
| 24 | 434 | 620 | 381 | 478 |
| 48 | 60.3 | 77.8 | 71.6 | 69.9 |
| 72 | BLOQ | BLOQ | BLOQ | ND |
| Animal Weight (kg) | 4.2 | 4.9 | 5.0 | 4.7 |
| Dose (mg/kg) | 11.9 | 10.2 | 10.0 | 10.7 |
| Cₘₐₓ (ng/mL) | 42800 | 49100 | 22000 | 37967 |
| tₘₐₓ (hr) | 2.0 | 2.0 | 2.0 | 2.0 |
| t_{1/2} (hr) | 8.0 | 8.1 | 10.2 | 8.7 |
| AUCₐₗₗ (hr * ng/mL) | 205729 | 211631 | 87668 | 168343 |
| Dose Normalized Values | | | | |
| D_{nzed} AUCₐₗₗ | 17281 | 20740 | 8767 | 15596 |
| (hr * ng/mL/mg/kg) | | | | |

### Example 34

Formulation #2 (“Alcohol Formulation”) and Formulation #3 (“Tween-80

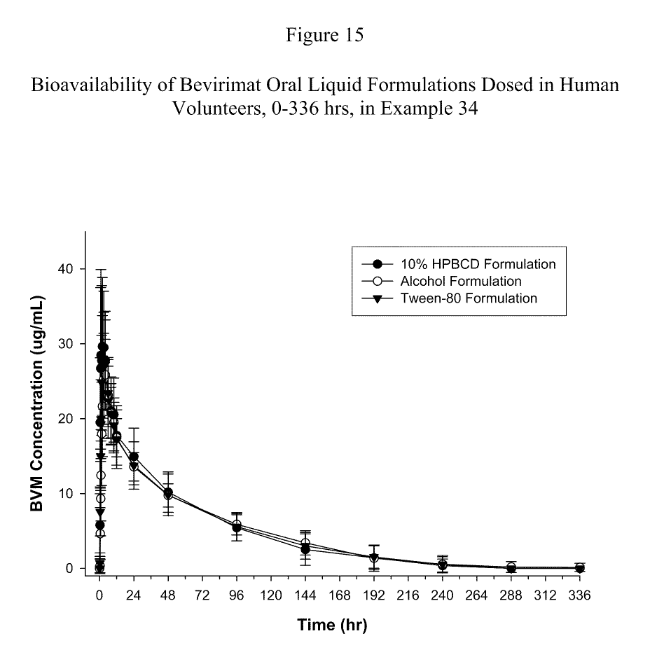

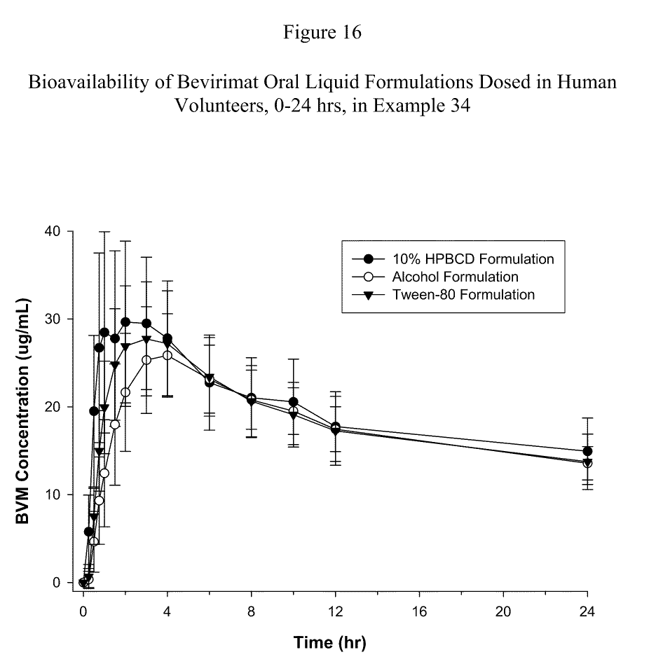

Formulation”) were tested in healthy human volunteers and compounded on site at the pharmacy unit, just before dosing. This was a single dose, bioequivalency study and all oral formulations were dosed at a concentration of 12.5 mg bevirimat free acid equivalents/mL and a dose volume of 16 mL, while the bevirimat dimeglumine solution in 10% (w/v) HPβCD vehicle (reference standard) and water were compounded at a concentration of 3.3 mg bevirimat free acid equivalents/mL and dose volume of 60 mL. The unit dose compositions of Formulation #2 and Formulation #3 are as described in Table 2 (Example 9) and Table 3 (Example 10), respectively. The results of this study are shown graphically in FIGS. 15 and 16, and in Table 19.

TABLE 19
Pharmacokinetic Parameters of Bevirimat Oral Solutions Tested in Healthy Human Volunteers

| | | AUC_{INF} | Cmax | C₂₄ | T_{1/2} | Tₘₐₓ | % F | | |
|---|---|---|---|---|---|---|---|---|---|
| Formulation | | hr * ug/mL | ug/mL | ug/mL | hr | hr | AUC_{INF} | Cₘₐₓ | C₂₄ |
| 10% | Mean | 1630.0 | 32.2 | 15.0 | 59.1 | 2.1 | | | |
| HPBCD | SD | 501.3 | 8.9 | 3.8 | 14.9 | 0.9 | | | |
| | CV % | 30.8 | 27.7 | 25.3 | 25.2 | 42.2 | | | |
| Alcohol | Mean | 1659.8 | 27.2 | 13.6 | 66.0 | 3.9 | 102% | 84% | 91% |
| (Formulation | SD | 434.9 | 4.9 | 1.9 | 20.2 | 1.0 | | | |
| #2) | CV % | 26.2 | 17.9 | 14.0 | 30.7 | 26.6 | | | |
| Tween-80 | Mean | 1630.7 | 29.5 | 13.7 | 62.4 | 2.9 | 100% | 92% | 92% |
| (Formulation | SD | 503.1 | 5.9 | 3.2 | 15.9 | 1.4 | | | |
| #3) | CV % | 30.9 | 19.9 | 23.0 | 25.4 | 46.8 | | | |
| Water | Mean | 1395.1 | 26.6 | 13.3 | 54.9 | 3.4 | 86% | 83% | 89% |
| | SD | 466.2 | 8.0 | 4.3 | 9.7 | 1.0 | | | |
| | CV % | 33.4 | 29.9 | 32.6 | 17.7 | 29.6 | | | |

The pharmacokinetic profiles of the “Alcohol Formulation” and “Tween-80 Formulation” were nearly identical to those of the reference bevirimat oral solution in 10% HPβCD formulation (FIGS. 15 and 16) and analysis of PK parameters indicated that the solutions were bioequivalent with F values of 102% and 100% for “Alcohol Formulation” and “Tween-80 Formulation”, respectively (Table 19). A bevirimat oral solution in water also had 86% relative oral bioavailability and was bioequivalent to both the reference bevirimat oral solution and Formulation #2 and Formulation #3.

### Example 35

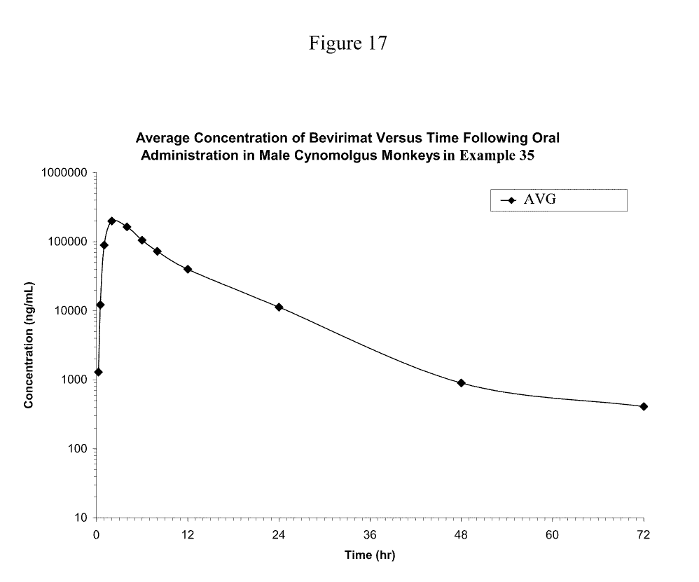

Male Cynomolgus monkeys were administered a bevirimat dimeglumine liquid formulation comprising 12.5 mg bevirimat free acid equivalents/mL in 40% (v/v) PG, 20% (v/v) glycerin, 5% (w/v) Vitamin ETPGS, 0.25% (v/v) Tween80, 0.20% (w/v) Plasdone C15 containing 10 mM sodium phosphate buffer, pH 8.0. Each monkey received 200 mg bevirimat free acid equivalents administered by oral gavage. Results are shown below in Table 20. FIG. 17 depicts the average plasma concentration of bevirimat versus time.

TABLE 20
Individual and Average Plasma Concentrations (ng/mL) for
Bevirimat Following Oral Administration into Male
Cynomolgus Monkeys (200 mg/monkey)

| | Monkey # | | |
|---|---|---|---|
| | 181 | 182 | AVG |
| Time (hr) | | | |
| 0 | BLOQ | BLOQ | ND |
| (pre-dose) | | | |
| 0.25 | 257 | 2340 | 1299 |
| 0.50 | 23100 | 1400 | 12250 |
| 1.0 | 108000 | 71100 | 89550 |
| 2.0 | 225000 | 174000 | 199500 |
| 4.0 | 163000 | 166000 | 164500 |
| 6.0 | 107000 | 104000 | 105500 |
| 8.0 | 70300 | 75400 | 72850 |
| 12 | 47500 | 32800 | 40150 |
| 24 | 20500 | 2100 | 11300 |
| 48 | 1350 | 455 | 903 |
| 72 | 739 | 80.6 | 410 |
| Animal Weight (kg) | 4.6 | 5.0 | 4.8 |
| Volume Administered (mL) | 18.4 | 20.0 | 19.2 |
| Dose (mg/kg) | 50.0 | 50.0 | 50.0 |
| Cₘₐₓ (ng/mL) | 225000 | 174000 | 199500 |
| tₘₐₓ (hr) | 2.0 | 2.0 | 2.0 |
| t_{1/2} (hr) | 8.3 | 10.2 | 9.2 |
| AUCₐₗₗ (hr * ng/mL) | 1968395 | 1393722 | 1681058 |
| Dose Normalized Values | | | |
| D_{nzed} AUCₐₗₗ | 39368 | 27874 | 33621 |
| (hr * ng/mL/mg/kg) | | | |

Cₘₐₓ: Maximum plasma concentration;
tₘₐₓ: Time of maximum plasma concentration;
t_{1/2}: Half-life, time points in bold were used in the calculation of the half life;
AUCₐₗₗ: Area Under the Curve, calculated using all data points;
D_{nzed}: Dose normalized, calculated by dividing the AUC by the dose;
BLOQ: Below the limit of quantitation;
ND: Not Determined

No adverse reactions were observed in this study. Animals in this study received a 4 mL/kg dose instead of the intended 4 mL/animal dose Immediately following dosing, animals received 8 mL of water.

### Example 36

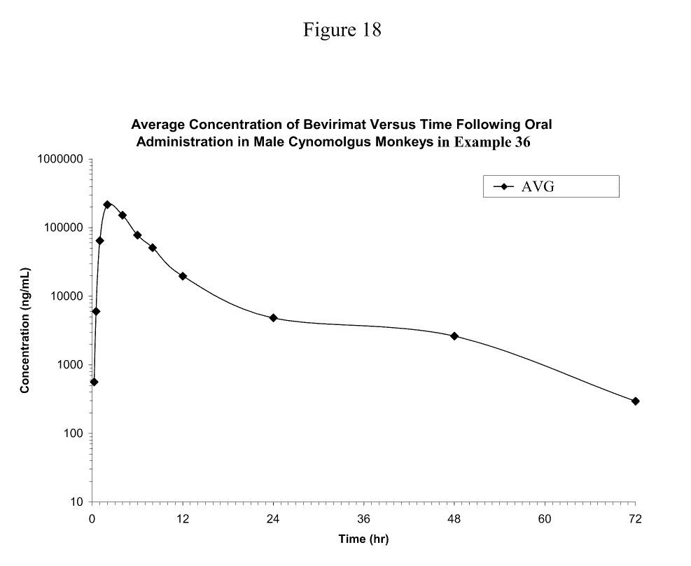

Male Cynomolgus monkeys were administered a bevirimat dimeglumine liquid formulation comprising 12.5 mg bevirimat free acid equivalents/mL in 30% (v/v) PG, 10% (v/v) glycerin, 5% (w/v) Vitamin ETPGS, and 15% (v/v) ethanol, containing 10 mM sodium phosphate buffer, pH 8.0. Each monkey received 200 mg bevirimat free acid equivalents administered by oral gavage. Results are shown below in Table 21. FIG. 18 depicts the average plasma concentration of bevirimat versus time.

TABLE 21
Individual and Average Plasma Concentrations (ng/mL) for
Bevirimat Following Oral Administration into Male
Cynomolgus Monkeys (200 mg/monkey)

| | Monkey # | | |
|---|---|---|---|
| | 185 | 186 | AVG |
| Time (hr) | | | |
| 0 | BLOQ | BLOQ | ND |
| (pre-dose) | | | |
| 0.25 | 1040 | 89.0 | 565 |
| 0.50 | 10300 | 1740 | 6020 |
| 1.0 | 88400 | 41200 | 64800 |
| 2.0 | 202000 | 232000 | 217000 |
| 4.0 | 166000 | 138000 | 152000 |
| 6.0 | 79400 | 76300 | 77850 |
| 8.0 | 53400 | 49200 | 51300 |
| 12 | 15600 | 23700 | 19650 |
| 24 | 2180 | 7500 | 4840 |
| 48 | 547 | 4700 | 2624 |
| 72 | 117 | 474 | 296 |
| Animal Weight (kg) | 5.4 | 4.7 | 5.1 |
| Volume Administered (mL) | 21.6 | 18.8 | 20.2 |
| Dose (mg/kg) | 50.0 | 50.0 | 50.0 |
| Cₘₐₓ (ng/mL) | 202000 | 232000 | 217000 |
| tₘₐₓ (hr) | 2.0 | 2.0 | 2.0 |
| t_{1/2} (hr) | 11.4 | 10.1 | 10.8 |
| AUCₐₗₗ (hr * ng/mL) | 1202995 | 1398863 | 1300929 |
| Dose Normalized Values | | | |
| D_{nzed} AUCₐₗₗ | 24060 | 27977 | 26019 |
| (hr * ng/mL/mg/kg) | | | |

Cₘₐₓ: Maximum plasma concentration;
tₘₐₓ: Time of maximum plasma concentration;
t_{1/2}: Half-life, time points in bold were used in the calculation of the half life;
AUCₐₗₗ: Area Under the Curve, calculated using all data points;
D_{nzed}: Dose normalized, calculated by dividing the AUC by the dose;
BLOQ: Below the limit of quantitation;
ND: Not Determined

No adverse reactions were observed in this study. Animals in this study received a 4 mL/kg dose instead of the intended 4 mL/animal dose. Immediately following dosing, animals received 8 mL of water.

### Example 37

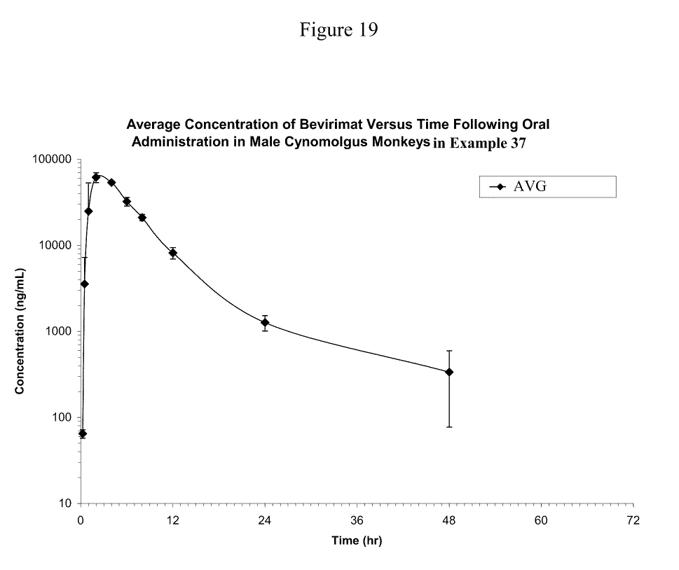

Male Cynomolgus monkeys were administered a bevirimat dimeglumine liquid formulation comprising 12.5 mg bevirimat free acid equivalents/mL in 40% (v/v) PG, 20% (v/v) glycerin, 5% (w/v) Vitamin ETPGS, 0.25% (v/v) Tween80, 0.20% (w/v) Plasdone C15 containing 10 mM sodium phosphate buffer, pH 8.0. Each monkey received 50 mg bevirimat free acid equivalents administered by oral gavage. Results are shown below in Table 22. FIG. 19 depicts the average plasma concentration of bevirimat versus time.

TABLE 22
Individual and Average Plasma Concentrations (ng/mL) for Bevirimat
Following Oral Administration into Male Cynomolgus Monkeys
(50 mg/Monkey)

| | Monkey # | | | | |
|---|---|---|---|---|---|
| | 181 | 182 | 184 | AVG | SD |
| Time (hr) | | | | | |
| 0 (pre-dose) | BLOQ | BLOQ | BLOQ | ND | ND |
| 0.25 | 69.8 | 56.3 | 67.8 | 64.6 | 7.29 |
| 0.50 | 409 | 2660 | 7590 | 3553 | 3673 |
| 1.0 | 1010 | 17400 | 56100 | 24837 | 28288 |
| 2.0 | 52700 | 63500 | 68500 | 61567 | 8075 |
| 4.0 | 55100 | 54100 | 51600 | 53600 | 1803 |
| 6.0 | 31500 | 36300 | 29000 | 32267 | 3710 |
| 8.0 | 22300 | 21800 | 19000 | 21033 | 1779 |
| 12 | 8580 | 9150 | 6750 | 8160 | 1254 |
| 24 | 1320 | 1490 | 988 | 1266 | 255 |
| 48 | 635 | 174 | 200 | 336 | 259 |
| 72 | 112 | BLOQ | BLOQ | ND | ND |
| Animal | 4.5 | 4.9 | 5.3 | 4.9 | 0.4 |
| Weight (kg) | | | | | |
| Dose | 11.1 | 10.2 | 9.4 | 10.2 | 0.8 |
| (mg/kg) | | | | | |
| Cₘₐₓ | 55100 | 63500 | 68500 | 62367 | 6772 |
| (ng/mL) | | | | | |
| tₘₐₓ (hr) | 4.0 | 2.0 | 2.0 | 2.7 | 1.2 |
| t_{1/2} (hr) | 8.6 | 5.3 | 5.4 | 6.4 | 1.9 |
| AUCₐₗₗ | 429062 | 459708 | 442472 | 443747 | 15362 |
| (hr * ng/mL) | | | | | |
| Dose | | | | | |
| Normalized | | | | | |
| Values | | | | | |
| D_{nzed} AUCₐₗₗ | 38616 | 45051 | 46902 | 43523 | 4350 |
| (hr * ng/mL/ | | | | | |
| mg/kg) | | | | | |

Cₘₐₓ: Maximum plasma concentration;
tₘₐₓ: Time of maximum plasma concentration;
t_{1/2}: Half-life, time points in bold were used in the calculation of the half life;
AUCₐₗₗ: Area Under the Curve, calculated using all data points;
D_{nzed}: Dose normalized, calculated by dividing the AUC by the dose;
BLOQ: Below the limit of quantitation;
ND: Not Determined

No adverse reactions were observed in this study. Immediately following dosing of 4 mL of formulation, animals received 8 mL of water.

### Example 38

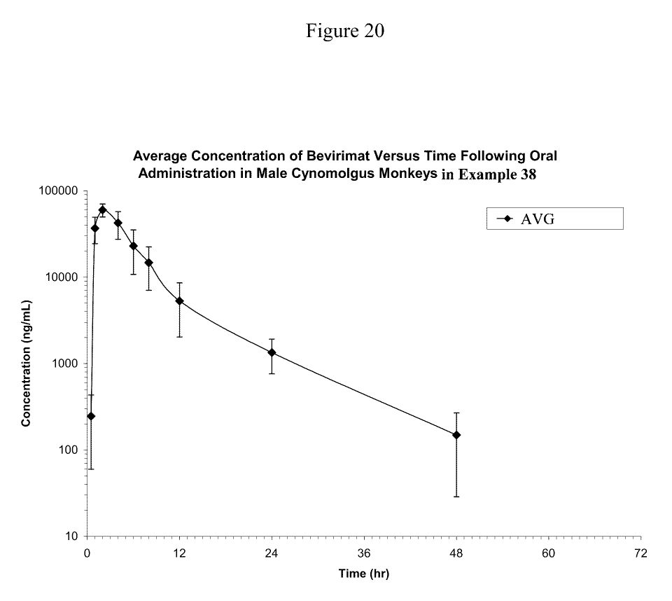

Male Cynomolgus monkeys were administered a bevirimat dimeglumine liquid formulation comprising 12.5 mg bevirimat free acid equivalents/mL in 30% (v/v) PG, 10% (v/v) glycerin, 5% (w/v) Vitamin ETPGS, and 15% (v/v) ethanol, containing 10 mM sodium phosphate buffer, pH 8.0. Each monkey received 50 mg bevirimat free acid equivalents administered by oral gavage. Results are shown below in Table 23. FIG. 20 depicts the average plasma concentration of bevirimat versus time.

TABLE 23
Individual and Average Plasma Concentrations (ng/mL) for Bevirimat
Following Oral Administration into Male Cynomolgus Monkeys (50 mg/
Monkey)

| | Monkey # | | | | |
|---|---|---|---|---|---|
| | 185 | 186 | 187 | AVG | SD |
| Time (hr) | | | | | |
| 0 (pre-dose) | BLOQ | BLOQ | BLOQ | ND | ND |
| 0.25 | BLOQ | 78.4 | BLOQ | ND | ND |
| 0.50 | 268 | 420 | 49.7 | 246 | 186 |
| 1.0 | 47500 | 40200 | 23100 | 36933 | 12524 |
| 2.0 | 58900 | 50500 | 70800 | 60067 | 10200 |
| 4.0 | 34500 | 33200 | 59800 | 42500 | 14996 |
| 6.0 | 18100 | 14000 | 37000 | 23033 | 12268 |
| 8.0 | 10600 | 10000 | 23600 | 14733 | 7685 |
| 12 | 3390 | 3450 | 9100 | 5313 | 3279 |
| 24 | 922 | 1100 | 2000 | 1341 | 578 |
| 48 | 62.6 | 97.8 | 286 | 149 | 120 |
| 72 | BLOQ | BLOQ | BLOQ | ND | ND |
| Animal | 5.5 | 4.7 | 4.9 | 5.0 | 0.4 |
| Weight (kg) | | | | | |
| Dose | 9.1 | 10.6 | 10.2 | 10.0 | 0.8 |
| (mg/kg) | | | | | |
| Cₘₐₓ | 58900 | 50500 | 70800 | 60067 | 10200 |
| (ng/mL) | | | | | |
| tₘₐₓ (hr) | 2.0 | 2.0 | 2.0 | 2.0 | 0 |
| t_{1/2} (hr) | 6.2 | 7.0 | 7.4 | 6.9 | 0.6 |
| AUCₐₗₗ | 306294 | 280224 | 503608 | 363375 | 122142 |
| (hr * ng/mL) | | | | | |
| Dose | | | | | |
| Normalized | | | | | |
| Values | | | | | |
| D_{nzed} | 33692 | 26341 | 49354 | 36462 | 11754 |
| AUCₐₗₗ | | | | | |
| (hr * ng/mL/ | | | | | |
| mg/kg) | | | | | |

Cₘₐₓ: Maximum plasma concentration;
tₘₐₓ: Time of maximum plasma concentration;
t_{1/2}: Half-life, time points in bold were used in the calculation of the half life;
AUCₐₗₗ: Area Under the Curve, calculated using all data points;
D_{nzed}: Dose normalized, calculated by dividing the AUC by the dose;
BLOQ: Below the limit of quantitation;
ND: Not Determined

No adverse reactions were observed in this study. Immediately following dosing of 4 mL of formulation, animals received 8 mL of water.

### Example 39

Liquid formulations A-E containing bevirimat-2NMG according to the present invention were compounded at 100 mL bench scale as shown in Table 24:

TABLE 24
Formulas for Prototype Formulations

| | Composition of Bevirimat Oral Solutions | | | | |
|---|---|---|---|---|---|
| | A | B | C | D | E |
| Component | w/v % | w/v % | w/v % | w/v % | w/v % |
| Ethanol | 11.85 | 12.38 | 12.97 | 13.56 | 14.15 |
| Propylene Glycol | 31.20 | 32.63 | 34.19 | 35.74 | 37.30 |
| Glycerin | 12.60 | 13.23 | 13.86 | 14.49 | 15.12 |
| 0.2 M Sodium Phosphate | 25.00 | 26.25 | 27.50 | 28.75 | 30.00 |
| Buffer, pH 7.9 | | | | | |
| Menthol (liquid) | 0.50 | 0.53 | 0.55 | 0.58 | 0.60 |
| Xylitol | 5.00 | 5.25 | 5.50 | 5.75 | 6.00 |
| Bevirimat 2-NMG | 2.09 | 2.09 | 2.09 | 2.09 | 2.09 |
| QS Water | 16.65 | 13.43 | 9.41 | 5.19 | 0.00 |

The physical and chemical stability of the Formulations A-E were tested according the stability protocol as follows. Stability samples were aliquotted into either 4 mL glass vials sealed with teflon-lined, screw closures or 10 mL glass vials sealed with Teflon-lined, silicon rubber, crimped closures, and set at room temperature (23° C.±3° C.), 40° C.±3° C. (water bath), and 60° C.±3° C. (circulating oven). Samples at 60° C. (stress stability conditions) were observed at approximately weekly intervals for the first two months for signs of physical instability, e.g., precipitation, and samples maintained at 60° C. and room temperature were analyzed for potency and related substances, using a qualified HPLC method, at approximately monthly intervals.

Observations on the physical stability of the Formulations A-E and D-L Menthol above are detailed in Table 25. All solutions were clear, physically stable and liquid at room temperature (RT), 40° C., and 60° C., for 71-89 days post-compounding (see Table 25). Formulation A had physical stability for at least 88 days.

TABLE 25
Observations on Physical Stability of Oral Bevirimat Solutions at
Room Temperature and 60° C.

| Formulation | Days on Stability |
|---|---|
| Formulation B | 87 |
| Formulation C | 87 |
| Formulation D | 87 |
| Formulation D-L Menthol | 71 |
| Formulation E | 89 |

Formulations A-E, and D-L Menthol were also tested for potency and related impurities using a validated HPLC method and the results are shown in Table 26. Potency was measured at room temperature (RT) and 60° C. and the difference (% Delta) was calculated (Table 26).

TABLE 26
Chemical Stability of Oral Bevirimat Solutions at 60° C.

| | Bevirimat Potency and Related Substances | | | | | |
|---|---|---|---|---|---|---|
| | mg BVM | mg BVM | | % Total Area | | Duration |
| | FAE/mL | FAE/mL | | | 15 min | at 60 Deg C. |
| Formulation | RT | 60 Deg C. | % Delta | 15 min RT | 60 Deg C. | Days |
| Formulation A | 11.5 | 11.9 | 3.7% | | | 88 |
| Formulation B | 12.0 | 11.6 | −3.0% | | 0.107 | 44 |
| Formulation C | 12.0 | 11.8 | −1.6% | 0.096 | | 44 |
| Formulation D | 11.9 | 11.9 | −0.3% | | 0.112 | 44 |
| Formulation D | 12.2 | 11.8 | −3.4% | 0.091 | 0.101 | 29 |
| L Menthol | | | | | | |
| Formulation E | 11.4 | 11.2 | −1.9% | 0.117 | 0.118 | 46 |

Target potency was 12.5 mg bevirimat free acid equivalents (BVM FAE)/mL. No significant changes in potency were observed. All the tested formulations had trace quantities of impurities (<0.1%) eluting at 15 min that did not increase in quantity with increasing temperature.

### Example 40

Liquid Formulations AA-EE containing bevirimat-2NMG according to the present invention were compounded at 1 L bench scale from 100 mL bench scale (10× scaleup) as shown in Table 27.

TABLE 27
Formulas for One Liter Scale-Up Formulations

| | Composition of BVM Oral Solutions | | | | | | |
|---|---|---|---|---|---|---|---|
| | AA | BB | CC | DD | DD-L Menthol | DD Mint | EE |
| Component | w/v % | w/v % | w/v % | w/v % | w/v % | w/v % | w/v % |
| Ethanol | 11.85 | 12.38 | 12.97 | 13.56 | 13.57 | 13.56 | 14.15 |
| Propylene Glycol | 31.20 | 32.63 | 34.19 | 35.74 | 35.74 | 35.74 | 37.30 |
| Glycerin | 12.60 | 13.23 | 13.86 | 14.49 | 14.49 | 14.49 | 15.12 |
| 0.2 M Sodium Phosphate | 25.00 | 26.25 | 27.50 | 28.75 | 28.75 | 28.75 | 30.00 |
| Buffer, pH 7.9 | | | | | | | |
| Menthol (liquid) | 0.50 | 0.53 | 0.55 | 0.58 | | | 0.60 |
| L Menthol (crystal) | | | | | 0.5 | | |
| Mint (liquid) | | | | | | 0.23 | |
| Xylitol | 5.00 | 5.25 | 5.50 | 5.75 | 5.75 | 5.75 | 6.00 |
| Bevirimat 2-NMG | 2.09 | 2.09 | 2.09 | 2.09 | 2.09 | 2.09 | 2.09 |
| QS Water | 16.65 | 13.43 | 9.41 | 5.19 | 5.19 | 5.19 | 0.00 |

The physical and chemical stability of Formulations AA-EE, DD-L Menthol, and DD Mint were tested according to the protocol described in Example 39. Observations on the physical stability of the Formulations AA-EE, DD-L Menthol, and DD Mint above are detailed in Table 28. All solutions were clear, physically stable and liquid at room temperature, 40° C. and 60° C., for 68-88 days post-compounding (Table 28).

TABLE 28
Physical Stability of One Liter Oral Bevirimat Solutions at 60° C.

| Formulation | Days on Stability |
|---|---|
| Formulation AA | 81 |
| Formulation BB | 84 |
| Formulation CC | 88 |
| Formulation DD | 85 |
| Formulation DD-L Menthol | 71 |
| Formulation DD Mint | 68 |
| Formulation EE | 76 |

The solutions were tested for potency and related impurities using a validated HPLC method and the results are shown in Table 29:

TABLE 29
Chemical Stability of Oral Bevirimat Solutions at 60° C.

Bevirimat Potency
mg BVM FAE/mL

| Formulation | RT | 60 Deg C. | % Delta | |
|---|---|---|---|---|
| Formulation AA | 12.2 | 11.9 | −2.6% | 31 |
| Formulation BB | 12.1 | 12.3 | 2.2% | 37 |
| Formulation CC | 12.7 | 12.5 | −1.8% | 39 |
| Formulation DD | 12.3 | 12.1 | −1.5% | 38 |
| Formulation DD-L | 12.2 | 12.4 | 1.8% | 28 |
| Menthol | | | | |
| Formulation DD Mint | 12.7 | 13.4 | 5.6% | 22 |
| Formulation EE | 13.6 | 12.3 | −9.7% | 30 |

Potency was measured at room temperature (RT) and 60° C. and the difference (% Delta) was calculated (Table 29). Target potency was 12.5 mg bevirimat free acid equivalents/mL. No significant changes in potency were observed with the exception of Formulation DD Mint which had a 9.7% decrease in potency. No formulations showed signs of betulinic acid or other bevirimat related impurities.

### Example 41

Useful buffers that can be employed in the liquid pharmaceutical compositions of the present invention include those listed in Table 30 below.

TABLE 30
Suitable buffers that do not react with bevirimat

| effective | | | |
|---|---|---|---|
| pH | pKa | | |
| range | 25° C. | buffer | Chemical name |
| 5.8-8.0 | 7.20 | phosphate (pK2) | |
| 6.0-8.0 | 6.35 | carbonate (pK1) | |
| 6.1-7.5 | 6.76 | PIPES | 1,4-Piperazinediethanesulfonic Acid |
| 6.2-7.8 | 6.95 | imidazole | 1,3-Diaza-2,4-cyclopentadiene |
| 6.3-9.5 | 6.80, | BIS-TRIS | 1,3-bis(tris(hydroxymethyl)methylamino) propane |
| | 9.00 | propane | |
| 6.4-7.8 | 7.09 | BES | N,N-Bis(2-Hydroxyethyl)-2-Aminoethanesulfonic Acid |
| 6.5-7.9 | 7.14 | MOPS | 3-Morpholino-2-Hydroxypropanesulfonic Acid |
| 6.8-8.2 | 7.48 | HEPES | 4-(2-Hydroxyethyl)-1-piperazineethanesulfonic acid |
| 6.8-8.2 | 7.40 | TES | |
| 7.0-8.2 | 7.52 | DIPSO | 3-[N,N-Bis(2-hydroxyethyl)amino]-2-hydroxypropanesulfonic acid |
| 7.0-8.2 | 7.61 | TAPSO | 3-[N-Tris(hydroxymethyl)methylamino]-2-hydroxypropane-sulfonic |
| | | | acid |
| 7.0-8.3 | 7.76 | triethanolamine | 2-[Bis-(2-hydroxy-ethyl)-amino]-ethanol |
| | | (TEA) | |
| 7.0-9.0 | 0.91, | pyrophosphate | |
| | 2.10, | | |
| | 6.70, | | |
| | 9.32 | | |
| 7.1-8.5 | 7.85 | HEPPSO | N-(2-Hydroxyethyl)piperazine-N′-(2-hydroxypropane-3-sulfonic acid) |
| 7.2-8.5 | 7.78 | POPSO | Piperazine-1,4-Bis(2-Hydroxypropane Sulfonic Acid) |
| 7.4-8.8 | 8.05 | tricine | N-[2-Hydroxy-1,1-bis(hydroxymethyl)ethyl]glycine |
| 7.6-9.0 | 8.26 | BICINE | N,N-Bis(2-hydroxyethyl)glycine |
| 7.6-9.0 | 8.30 | HEPBS | N-(2-Hydroxyethyl)piperazine-N′-(4-butanesulfonic acid) |
| 7.7-9.1 | 8.40 | TAPS | 3-[(Tris(hydroxymethyl)methyl)amino]-1-propanesulfonic acid |
| 8.2-9.6 | 8.90 | TABS | N-Tris(hydroxymethyl)methyl-4-aminobutanesulfonic Acid |
| 8.3-9.7 | 9.00 | AMPSO | N-(1,1-Dimethyl-2-hydroxyethyl)-3-amino-2- |
| | | | hydroxypropanesulphonic acid |
| 8.5-10.2 | 9.23, | borate | |
| | 12.74, | | |
| | 13.80 | | |

### Example 42

Suitable exemplary flavoring agents for use in the liquid pharmaceutical compositions of the present invention are provided in Tables 31 and 32 below.

TABLE 31
Flavoring agents suitable for use in liquid bevirimat oral dosage forms

| | | | Starting Usage |
|---|---|---|---|
| Flavoring Agent | Supplier | Catalog# | Level |
| Menthol, natural | Virginia Dare | N 22760 | 0.25% (v/v) |
| Mint, natural | Virginia Dare | H27299 | 0.1% (v/v) |
| &artificial | | | |
| Mint, Ext Nat K | Virginia Dare | HD06 | 0.3% (v/v) |
| Coffee Dart | Virginia Dare | 26477 | 0.5% (v/v) |
| Roast, N&A | | | |
| Strawberry, N&A | Virginia Dare | 958K | 0.5% (v/v) |
| wild | | | |
| Raspberry, | Virginia Dare | 998K | 0.3% (v/v) |
| artificial | | | |
| Wintergreen, | Virginia Dare | KQ75 | 0.3% (v/v) |
| natural Ext K | | | |
| Spearmint, N&A | Virginia Dare | L03 | 0.1% (v/v) |
| Banana tropical, | Virginia Dare | 18214K | 0.25% (v/v) |
| N&A FF | | | |
| L-Menthol, FCC | Spectrum Chemical | M1360 | 0.1% (w/v) |
| grade, powder | | | |

TABLE 32
Flavoring agents suitable for use in liquid bevirimat oral dosage forms

| | | | Upper Usage |
|---|---|---|---|
| Flavoring Agent | Supplier | Catalog# | Level |
| Peach, N&A | FONA | 889.115/WC | 0.4% (v/v) |
| Orange, natural | Mastertaste | 680262-PA | 0.75% (v/v) |
| WONF | | | |
| Orange Vanilla, | Virginia Dare | 17898 | 0.5% (v/v) |
| artificial | | | |
| Dulce de Leche | Virginia Dare | 19322K | 0.75% (v/v) |
| Mixed Berry, | Ungerer | F09457 | N/A |
| N&A | | | |
| Mixed berry, Nat | Givaudan | 522474 | N/A |
| WONF | | | |
| Stawberry, N&A | David Michael | 25253 | N/A |
| Stawberry, N&A | Ungerer | FN8378 | N/A |
| Red punch, N&A | FONA | 852.330WC | N/A |
| Banana, natural | Givaudan | 522433 | N/A |
| WONF | | | |
| Stawberry/ | Givaudan | 522503 | N/A |
| Banana, natural | | | |
| WONF | | | |
| Peach/Mango, | David Michael | 29860 | N/A |
| N&A | | | |
| Coffee, N&A | Ungerer | FP9627 | N/A |
| Mocha, artificial | David Michael | 24106 | N/A |

As various changes could be made in the above formulations and methods without departing from the scope of the invention, it is intended that all matter contained in the above description be interpreted as illustrative and not in a limiting sense. All patents, patent applications, and publications cited herein are fully incorporated by reference herein in their entirety.

What is claimed is:

1. An orally deliverable liquid pharmaceutical composition, comprising a bevirimat drug substance, propylene glycol, ethanol, and glycerin.

2. The composition of claim 1, wherein the weight/volume percent ratio of ethanol:propylene glycol:glycerin is about 1:from about 2 to about 4:about 1.

3. The composition of claim 1, comprising from about 26 to about 38 w/v-% of propylene glycol, from about 10 to about 15 w/v-% of ethanol, and from about 10 to about 17 w/v-% of glycerin.

4. The composition of claim 1, further comprising buffer that provides a pH of from about 7.0 to about 9.0.

5. The composition of claim 4, wherein the final molarity of said buffer in said composition is from about 0.01M to about 0.4M.

6. The composition of claim 4, wherein said buffer is phosphate buffer.

7. The composition of claim 6, wherein said phosphate buffer is sodium phosphate buffer.

8. The composition of claim 7, wherein said sodium phosphate buffer is 0.1-0.2M sodium phosphate buffer, pH 7.9.

9. The composition of claim 1, wherein the bevirimat drug substance is bevirimat dimeglumine.

10. The composition of claim 1, wherein the bevirimat drug substance is bevirimat dipotassium.

11. The composition of claim 1, wherein the bevirimat drug substance is bevirimat disodium.

12. The composition of claim 9, comprising from about 0.01 to about 0.42 w/v-% of bevirimat dimeglumine

13. The composition of claim 9, wherein the bevirimat dimeglumine is present in a therapeutically effective amount.

14. The composition of claim 13, wherein the bevirimat dimeglumine is present in a total amount of from about 1 to about 25 mg/mL free acid equivalents of bevirimat.

15. The composition of claim 1, further comprising one or more flavoring agents.

16. The composition of claim 15, wherein said one or more flavoring agents are selected from the group consisting of mint, menthol, peach, Dulce de Leche, coffee, orange, orange/vanilla, strawberry, peach/mango, punch, chocolate, mixed perry, mocha, strawberry/banana, and banana.

17. The composition of claim 1, further comprising a sweetener.

18. The composition of claim 17, wherein said sweetener is xylitol.

19. The composition of claim 1, wherein at least about 85% of the bevirimat drug substance is present in said composition in dissolved or solubilized form.

20. The composition of claim 1, wherein substantially all of the bevirimat drug substance is dissolved or solubilized in said composition.

21. The composition of claim 1, wherein said composition is provided in one or more dose units each comprising about 100 mg to about 1000 mg of the bevirimat drug substance.

22. The composition of claim 1, further comprising a polymer.

23. The composition of claim 22, wherein said polymer is Vitamin E TPGS.

24. The composition of claim 1, wherein the liquid pharmaceutical composition is selected from the group consisting of a solution, a suspension, an emulsion, a gel, a syrup, and an elixir.

25. The composition of claim 24, wherein the liquid pharmaceutical composition is an emulsion, and the emulsion is a microemulsion or a nanoemulsion.

26. A method of treating a medical condition or disorder in a subject where treatment with a HIV-1 replication inhibitor is indicated, comprising orally administering to the subject a composition according to claim 1.

27. Use of a bevirimat drug substance in the manufacture of an orally deliverable liquid pharmaceutical composition for treating HIV infection and related diseases in a human patient.
